(19) United States
(12) Patent Application Publication
(10) Pub. No.: US 2025/0360081 A1
(43) Pub. Date: Nov. 27, 2025

(54) ORAL DRUG DOSAGE FORMS FOR STOMACH RETENTION

(71) Applicant: Triastek, Inc., Nanjing (CN)

(72) Inventors: Xianghao ZUO, Nanjing (CN); Feihuang DENG, Nanjing (CN); Qing LUO, Nanjing (CN); Peizhan YANG, Nanjing (CN); Fuqiang LIU, Nanjing (CN); Ang ZHAO, Nanjing (CN); Ruru ZHANG, Nanjing (CN); Xiaoling LI, Dublin, CA (US); Senping CHENG, Nanjing (CN)

(21) Appl. No.: 19/122,172

(22) PCT Filed: Oct. 18, 2023
(86) PCT No.: PCT/CN2023/125190

§ 371 Date: Apr. 17, 2025

(30) Foreign Application Priority Data
Oct. 18, 2022 (WO) PCT/CN2022/125982

(51) Int. Cl. A61K 9/00; A61K 47/02; A61K 47/32; A61K 47/38

(52) U.S. Cl. CPC A61K 9/0065; A61K 47/02; A61K 47/32; A61K 47/38

(57) ABSTRACT

Provided herein are drug dosage forms designed to provide a desired retention in an individual based on geometric differences between a pre-administration and post-administration state of the dosage form. In certain aspects, provided are oral drug dosage forms comprising a swellable and expandable material configured, upon swelling or expanding, to drive expansion of the overall size of the oral drug dosage forms such that the oral drug dosage forms are retained in the stomach for a desired period of time. In certain other aspects, provided is directed to components useful for the oral drug dosage forms taught herein. In certain other aspects, provided is directed to methods of designing, methods of making, such as involving three-dimensional (3D) printing, injection molding, ultrasonic welding, or any combination thereof, commercial batches, and methods of delivering a drug to an individual comprising administering an oral drug dosage form taught herein.

### CROSS-REFERENCE TO RELATED APPLICATIONS

This application claims prior to and the benefit of PCT/CN2022/125982, filed on Oct. 18, 2022, which is incorporated by reference herein in its entirety.

### TECHNICAL FIELD

The present disclosure, in some aspects, is directed to drug dosage forms designed to provide a desired retention in an individual based on geometric differences between a pre-administration and post-administration state of the dosage form. In certain aspects, provided are oral drug dosage forms comprising a swellable and expandable material configured, upon swelling or expanding, to drive expansion of the overall size of the oral drug dosage forms such that the oral drug dosage forms are retained in the stomach for a desired period of time. In certain other aspects, the present disclosure is directed to components useful for the oral drug dosage forms taught herein. In certain other aspects, the present disclosure is directed to methods of designing, methods of making, such as involving three-dimensional (3D) printing, injection molding, ultrasonic welding, or any combination thereof, commercial batches, and methods of delivering a drug to an individual comprising administering an oral drug dosage form taught herein.

### BACKGROUND

Conventional oral drug dosage forms, when administered to an individual, are subject to natural forces, such as the natural flow of fluids, semi-solids, and solids through the gastrointestinal system of the individual, and/or forces exerted by the individual, e.g., muscle contractions, or the individual's environment, e.g., gravity. As is the case with oral administration, this natural flow can vary between administrations of a drug dosage form to a specific individual, and across a population of individuals, based on fluctuating circumstances relative to when the drug dosage form was administered, such timing and size of a meal and/or drink, the meal and/or drink contents, and the current stomach phase and remaining duration. These circumstance create variability in, e.g., the anatomical location of drug delivery, bioavailability, safety profile, and efficacy of a drug dosage form that has a period of residency in the stomach, such as an oral drug dosage form.

### BRIEF SUMMARY

In certain aspects, provided is an oral drug dosage form configured for gastric retention, the oral drug dosage form comprising: a swellable material; a moveable arm configured such that at least a portion of the moveable arm is extendible beyond, or further away from, a body of the oral drug dosage form via a force that is provided by the swellable material; and the body comprising: a swellable material compartment configured to contain at least a portion of the swellable material; a directional channel and/or an orifice operably connected to the swellable material compartment, wherein the moveable arm comprises a contact element configured to interface with the swellable material, or a feature associated with the swellable material, in proximity to the directional channel and/or the orifice; and one or more fluid inlets operably connected to the swellable material compartment; and a drug, wherein the oral drug dosage form is configured to have a pre-administration state having a compact form and a post-administration state having an extended form providing the gastric retention, and wherein the extended form of the post-administration state of the oral drug dosage form is due to, at least in part, expansion of the swellable material in the presence of a gastrointestinal fluid.

In certain aspects, provided is a drug dosage form or medical device that stays in the body, the drug dosage form or medical device comprising: a swellable material; a moveable arm configured such that at least a portion of the moveable arm is extendible beyond, or rotate around a body of the drug dosage form or medical device via a force that is provided by the swellable material; and the body comprising: a swellable material compartment configured to contain at least a portion of the swellable material; wherein the swellable material compartment operably comprises one or more fluid inlets; a directional channel and an orifice operably connected to the swellable material compartment, wherein the moveable arm comprises a contact element configured to interface with the swellable material, or a feature associated with the swellable material, in proximity to the directional channel and the orifice; and a drug, wherein the drug dosage form or medical device is configured to have a pre-administration state having a compact form and a post-administration state having an extended form providing the stay in the body, and wherein the extended form of the post-administration state of the drug dosage form or medical device is due to, at least in part, expansion of the swellable material in the presence of a body fluid.

In some embodiments, wherein the feature associated with the swellable material comprises a plunger or a piston.

In some embodiments, wherein the movable arm or body comprises a pivot, and the movable arm is connected to the body by pivoting.

In some embodiments, wherein the swellable material compartment comprises a cap and a base, and the pivot of the movable arm is connected to the cap.

In some embodiments, the post-administration state of the oral drug dosage form occurs within 1 hour or less following administration of the oral drug dosage form to the individual.

In some embodiments, the gastric residence of the oral drug dosage form is about 8 hours to about 3 months.

In some embodiments, the swellable material expands at least about 1.2× in volume following exposure to a gastrointestinal fluid. In some embodiments, the swellable material has a volume expansion rate of at least about 1.2× in 30 minutes.

In some embodiments, the swellable material, upon swelling, at least in part conforms to the shape of the direction channel and/or the orifice, or a portion thereof.

In some embodiments, the swellable material, upon swelling, adopts a pre-determined shape and/or size.

In some embodiments, the swellable material in the swellable material compartment is of an amount of at least about 5 mg.

In some embodiments, the swellable material comprises sodium alginate (SA), hydroxypropyl cellulose (HPC), hydroxyethyl cellulose (HEC), hydroxypropyl methyl cellulose (HPMC), polyethylene oxide (PEO), polyvinyl alcohol (PVA), microcrystalline cellulose (MCC), croscarmellose sodium (CCNa), carboxymethylcellulose sodium (CMC-Na), polyvinylpolypyrrolidone (PVPP), carboxymethyl starch sodium (CMS-Na), polyethylene glycol (PEG), or a mixture thereof. In some embodiments, the swellable material further comprises a salt or a mixture of salts. In some embodiments, the salt is selected from the group consisting of a sodium salt, a magnesium salt, and a kali salt.

In some embodiments, the swellable material has a swelling volume of about 10 mm³ to about 50 mm³.

In some embodiments, the body comprises two or more pieces configured to form the body. In some embodiments, the body comprises one or more bases and one or more caps. In some embodiments, the body comprises two or more materials. In some embodiments, the body of the oral drug dosage form is a monolithic structure.

In some embodiments, the body delineates the outer bounds of the oral drug dosage form in a pre-administration state.

In some embodiments, the largest crossing dimension of the oral drug dosage form in a pre-administration state is 24 mm or less. In some embodiments, at least two perpendicular dimension of the oral drug dosage form in a post-administration state are 20 mm or more.

In some embodiments, the swellable material compartment is configured to substantially contain the swellable material in a pre-administration state. In some embodiments, the swellable material compartment and the contact element of the moveable arm substantially surround the swellable material.

In some embodiments, the directional channel is configured to direct movement of the moveable arm via the swellable material. In some embodiments, the directional channel comprises a curved-shaped channel. In some embodiments, the directional channel comprises a circular-shaped channel. In some embodiments, the directional channel comprises a square or rectangular-shaped channel. In some embodiments, the directional channel comprises a straight-shaped channel, an oval-shaped channel, or a capsule-shaped channel.

In some embodiments, the orifice is square or rectangular-shaped.

In some embodiments, the directional channel further comprises one or more fluid inlets. In some embodiments, at least one of the one or more fluid inlets comprises a semi-permeable material. In some embodiments, the at least one of the one or more fluid inlets is the semi-permeable material filling a pore formed by the body. In some embodiments, the one or more fluid inlets is a semi-permeable membrane at least partially wrapping around the swellable material. In some embodiments, at least one of the one or more fluid inlets is a pore.

In some embodiments, the body further forms a stop configured to engage with the moveable arm at an extended position. In some embodiments, the stop comprises a locking element configured to maintain the moveable arm at one or more extended positions.

In some embodiments, the moveable arm, or a portion thereof, is configured to slide within the directional channel. In some embodiments, the contact element of the moveable arm is configured to slide within the directional channel. In some embodiments, the moveable arm is configured to rotate on an axis.

In some embodiments, wherein the oral drug dosage form further comprises one or more moveable arms. In some embodiments, at least two of the moveable arms have a different movement mechanism. In some embodiments, at least two of the moveable arms have the same movement mechanism. In some embodiments, at least two of the moveable arms are configured such that at least a portion of each of the moveable arms is extendible beyond, or further away from, the body of the oral drug dosage form via a force that is provided by the swellable material.

In some embodiments, the oral drug dosage form further comprises a second swellable material contained, at least in part, in a second swellable material compartment formed by the body, wherein at least one of the moveable arms is configured such that at least a portion of the moveable arm is extendible beyond, or further away from, the body of the oral drug dosage form via a force that is provided by the second swellable material.

In some embodiments, the oral drug dosage form further comprises an erodible restrainer configured to inhibit extension of the moveable arm. In some embodiments, the erodible restrainer erodes within about 30 minutes following administration to an individual.

In some embodiments, the drug is located in one or more of: the moveable arm and the body. In some embodiments, the drug is selected from the group consisting of riociguat, aceclofenac, bicalutamide, carbamazepine, carvedilol, clotrimazole, cinnarizine, danazol, dapsone, estradiol, exetimibe glibenclamide, fenofibrate, griseofulvin, ibuprofen, itraconazole, ketoconazole, mefenamic acid, naproxen, nevirapine, nifedipine, nitrofurantoin, nomegestrol acetate, phenytoin sodium salt, piroxicam, praziquantel, rifampicin, sulfamethoxazole, trimethoprim, and verapramil hydrochloride.

In other aspects, provided herein is an oral drug dosage form configured for a gastric retention, the oral drug dosage form comprising: a swellable material; a moveable arm configured such that at least a portion of the moveable arm is extendible beyond, or further away from, a body of the oral drug dosage form via a force that is provided by the swellable material; the body comprising: a swellable material compartment configured to contain at least a portion of the swellable material; a directional channel operably connected to the swellable material compartment, wherein the moveable arm comprises a contact element configured to interface with the swellable material in proximity to the directional channel, wherein the swellable material compartment and the contact element of the moveable arm substantially surround the swellable material, and wherein the moveable arm, or a portion thereof, is configured to slide within the directional channel such that the moveable arm extends beyond, or further away from, the body of the oral drug dosage form along an axis based on the directional channel; one or more fluid inlets operably connected to the swellable material compartment; a stop configured to engage with the moveable arm at an extended position; and a drug, wherein the oral drug dosage form is configured to have a pre-administration state having a compact form and a post-administration state having an extended form providing the gastric retention, and wherein the extended form of the post-administration state of the oral drug dosage form is due to, at least in part, expansion of the swellable material in the presence of a gastrointestinal fluid. In some embodiments, the stop comprises a locking element configured to maintain the moveable arm at one or more extended positions. In some embodiments, the locking element is configured to maintain the moveable arm at a single position. In some embodiments, the locking element is configured to maintain the moveable arm at a plurality of progressing positions.

In other aspects, provided is an oral drug dosage form configured for a gastric retention, the oral drug dosage form comprising: a swellable material; a first moveable arm and a second movable arm, wherein the first moveable arm and the second moveable arm are configured such that at least a portion of each moveable arm is extendible beyond, or further away from, a body of the oral drug dosage form via a force that is provided by the swellable material, and wherein the first moveable arm and the second moveable arm extend in substantially opposite directions along an axis based on the directional channel; the body comprising: a swellable material compartment configured to contain at least a portion of the swellable material; a first directional channel and a second directional channel operably connected to the swellable material compartment, wherein the first moveable arm comprises a first contact element configured to interface with the swellable material in proximity to the first directional channel, wherein the second moveable arm comprises a second contact element configured to interface with the swellable material in proximity to the second directional channel, wherein the swellable material compartment and the first contact element of the first moveable arm and second contact element of the second moveable arm substantially surround the swellable material, and wherein the first moveable arm, or a portion thereof, is configured to slide within the first directional channel such that the first moveable arm extends beyond, or further away from, the body of the oral drug dosage form along the axis, and wherein the second moveable arm, or a portion thereof, is configured to slide within the second directional channel such that the second moveable arm extends beyond, or further away from, the body of the oral drug dosage form along the axis; one or more fluid inlets operably connected to the swellable material compartment; a first stop configured to engage with the first moveable arm at an extended position; a second stop configured to engage with the second moveable arm at an extended position; and a drug, wherein the oral drug dosage form is configured to have a pre-administration state having a compact form and a post-administration state having an extended form providing the gastric retention, and wherein the extended form of the post-administration state of the oral drug dosage form is due to, at least in part, expansion of the swellable material in the presence of a gastrointestinal fluid.

In other aspects, provided herein is an oral drug dosage form configured for a gastric retention, the oral drug dosage form comprising: a first swellable material; a first moveable arm, wherein the first moveable arm is configured such that at least a portion of the moveable arm is extendible beyond, or further away from, a body of the oral drug dosage form via a force that is provided by the first swellable material, and wherein the first moveable arm is configured to rotate on a first axis; a second swellable material; a second moveable arm, wherein the second moveable arm is configured such that at least a portion of the moveable arm is extendible beyond, or further away from, the body of the oral drug dosage form via a force that is provided by the second swellable material, and wherein the second moveable arm is configured to rotate on a second axis; the body comprising: a first swellable material compartment configured to contain at least a portion of the first swellable material; a first directional channel operably connected to the first swellable material compartment, wherein the first direction channel comprises a curved-shaped channel, wherein the first moveable arm comprises a first contact element configured to interface with the first swellable material in proximity to the first directional channel, wherein the first swellable material compartment and the first contact element of the first moveable arm substantially surround the first swellable material, and wherein the first moveable arm, or a portion thereof, is configured to rotate on the first axis by way of the first contact element sliding in the first direction channel such that the first moveable arm extends beyond, or further away from, the body of the oral drug dosage form, one or more fluid inlets operably connected to the first swellable material compartment; a second swellable material compartment configured to contain at least a portion of the second swellable material; a second directional channel operably connected to the second swellable material compartment, wherein the second direction channel comprises a curved-shaped channel, wherein the second moveable arm comprises a second contact element configured to interface with the second swellable material in proximity to the second directional channel, wherein the second swellable material compartment and the second contact element of the second moveable arm substantially surround the second swellable material, and wherein the second moveable arm, or a portion thereof, is configured to rotate on the second axis by way of the second contact element sliding in the second direction channel such that the second moveable arm extends beyond, or further away from, the body of the oral drug dosage form, one or more fluid inlets operably connected to the second swellable material compartment; a drug, wherein the oral drug dosage form is configured to have a pre-administration state having a compact form and a post-administration state having an extended form providing the gastric retention, and wherein the extended form of the post-administration state of the oral drug dosage form is due to, at least in part, expansion of the swellable material in the presence of a gastrointestinal fluid.

In other aspects, provided herein is an oral drug dosage form configured for a gastric retention, the oral drug dosage form comprising: a first swellable material; a first moveable arm and a second movable arm, wherein the first moveable arm and the second moveable arm are configured such that at least a portion of each moveable arm is extendible beyond, or further away from, a body of the oral drug dosage form via a force that is provided by the first swellable material, and wherein the first moveable arm and the second moveable arm extend in substantially opposite directions along an axis based on the directional channel; a second swellable material; a third moveable arm, wherein the third moveable arm is configured such that at least a portion of the third moveable arm is extendible beyond, or further away from, the body of the oral drug dosage form via a force that is provided by the second swellable material, and wherein the third moveable arm is configured to rotate on a first axis; a third swellable material; a fourth moveable arm, wherein the fourth moveable arm is configured such that at least a portion of the fourth moveable arm is extendible beyond, or further away from, the body of the oral drug dosage form via a force that is provided by the third swellable material, and wherein the fourth moveable arm is configured to rotate on a second axis; the body comprising: a first swellable material compartment configured to contain at least a portion of the first swellable material; a first directional channel and a second directional channel operably connected to the first swellable material compartment, wherein the first moveable arm comprises a first contact element configured to interface with the first swellable material in proximity to the first directional channel, wherein the second moveable arm comprises a second contact element configured to interface with the first swellable material in proximity to the second directional channel, wherein the first swellable material compartment and the first contact element of the first moveable arm and second contact element of the second moveable arm substantially surround the first swellable material, and wherein the first moveable arm, or a portion thereof, is configured to slide within the first directional channel such that the first moveable arm extends beyond, or further away from, the body of the oral drug dosage form along the axis, and wherein the second moveable arm, or a portion thereof, is configured to slide within the second directional channel such that the second moveable arm extends beyond, or further away from, the body of the oral drug dosage form along the axis; one or more fluid inlets operably connected to the first swellable material compartment; a first stop configured to engage with the first moveable arm at an extended position; a second stop configured to engage with the second moveable arm at an extended position; a second swellable material compartment configured to contain at least a portion of the second swellable material; a third directional channel operably connected to the second swellable material compartment, wherein the third direction channel comprises a curved-shaped channel, wherein the third moveable arm comprises a contact element configured to interface with the second swellable material in proximity to the third directional channel, wherein the second swellable material compartment and the contact element of the third moveable arm substantially surround the second swellable material, and wherein the third moveable arm, or a portion thereof, is configured to rotate on the first axis by way of the contact element sliding in the third direction channel such that the third moveable arm extends beyond, or further away from, the body of the oral drug dosage form, one or more fluid inlets operably connected to the third swellable material compartment; a third swellable material compartment configured to contain at least a portion of the third swellable material; a fourth directional channel operably connected to the third swellable material compartment, wherein the fourth direction channel comprises a curved-shaped channel, wherein the fourth moveable arm comprises a contact element configured to interface with the third swellable material in proximity to the fourth directional channel, wherein the third swellable material compartment and the contact element of the fourth moveable arm substantially surround the third swellable material, and wherein the fourth moveable arm, or a portion thereof, is configured to rotate on the second axis by way of the contact element sliding in the fourth direction channel such that the fourth moveable arm extends beyond, or further away from, the body of the oral drug dosage form, one or more fluid inlets operably connected to the third swellable material compartment; and a drug, wherein the oral drug dosage form is configured to have a pre-administration state having a compact form and a post-administration state having an extended form providing the gastric retention, and wherein the extended form of the post-administration state of the oral drug dosage form is due to, at least in part, expansion of the first swellable material, the second swellable material, and the third swellable material in the presence of a gastrointestinal fluid.

In other aspects, provided herein is an oral drug dosage form configured for a gastric retention, the oral drug dosage form comprising: a swellable material; a first moveable arm and a second movable arm, wherein the first moveable arm and the second moveable arm are configured such that at least a portion of each moveable arm is extendible beyond, or further away from, a body of the oral drug dosage form via a force that is provided by the swellable material, and wherein the first moveable arm and the second moveable arm are configured to rotate on a shared axis in opposite directions; the body comprising: a swellable material compartment configured to contain at least a portion of the swellable material; a first directional channel and a second directional channel operably connected to the swellable material compartment, wherein the first directional channel and the second directional channel are curved-shaped and configured around the shared axis, wherein the first moveable arm comprises a first contact element configured to interface with the swellable material in proximity to the first directional channel, wherein the first contact element is rudder-shaped and configured to travel in the first directional channel such that the first moveable arm rotates beyond, or further away from, the body of the oral drug dosage form relative to the shared axis, wherein the second moveable arm comprises a second contact element configured to interface with the swellable material in proximity to the second directional channel, wherein the second contact element is rudder-shaped and configured to travel in the second directional channel such that the second moveable arm rotates beyond, or further away from, the body of the oral drug dosage form relative to the shared axis, one or more fluid inlets operably connected to the swellable material compartment; a stop configured to engage with the first moveable arm and the second moveable arm at an extended position; a drug, wherein the oral drug dosage form is configured to have a pre-administration state having a compact form and a post-administration state having an extended form providing the gastric retention, and wherein the extended form of the post-administration state of the oral drug dosage form is due to, at least in part, expansion of the swellable material in the presence of a gastrointestinal fluid. In some embodiments, the stop is configured to engage with the first contact element of the first moveable arm and the second contact element of the second moveable arm at the extended position.

In other aspects, provided herein is an oral drug dosage form configured for a gastric retention, the oral drug dosage form comprising: a swellable material; a first moveable arm, a second movable arm, a third moveable arm, and a fourth moveable arm, wherein the first moveable arm, the second moveable arm, the third moveable arm, the fourth moveable arm are configured such that each arm rotates on an independent axis in a direction towards a perpendicularly situated plane via a force that is provided by the swellable material, a body comprising: a swellable material compartment configured to contain at least a portion of the swellable material; a directional channel and/or an orifice operably connected to the swellable material compartment, wherein each moveable arm comprises a contact element configured to interface with the swellable material in proximity to the directional channel and/or the orifice, and wherein the swellable material compartment, the contact elements of the moveable arms, and a cap substantially surround the swellable material; one or more fluid inlets operably connected to the swellable material compartment; a cap; a first stop, a second stop, a third stop, and a fourth stop configured to engage with the first moveable arm, second moveable arm, third moveable arm, and the fourth moveable arm, respectively, at an extended position; and a drug, wherein the oral drug dosage form is configured to have a pre-administration state having a compact form and a post-administration state having an extended form providing the gastric retention, and wherein the extended form of the post-administration state of the oral drug dosage form is due to, at least in part, expansion of the swellable material in the presence of a gastrointestinal fluid.

In other aspects, provided herein is an oral drug dosage form configured for a gastric retention, the oral drug dosage form comprising: a swellable material; a first moveable arm, a second movable arm, a third moveable arm, and a fourth moveable arm, wherein the first moveable arm, the second moveable arm, the third moveable arm, the fourth moveable arm are configured such that each arm rotates on an independent axis in a direction towards a perpendicularly situated plane via a force that is provided by the swellable material, a body comprising: a swellable material compartment configured to contain at least a portion of the swellable material; a directional channel and/or an orifice operably connected to the swellable material compartment, wherein each moveable arm comprises a contact element configured to interface with the swellable material in proximity to the directional channel and/or the orifice, and wherein the swellable material compartment and the contact elements of the moveable arms substantially surround the swellable material; one or more fluid inlets operably connected to the swellable material compartment; a first stop, a second stop, a third stop, and a fourth stop configured to engage with the first moveable arm, second moveable arm, third moveable arm, and the fourth moveable arm, respectively, at an extended position; and a drug, wherein the oral drug dosage form is configured to have a pre-administration state having a compact form and a post-administration state having an extended form providing the gastric retention, and wherein the extended form of the post-administration state of the oral drug dosage form is due to, at least in part, expansion of the swellable material in the presence of a gastrointestinal fluid. In some embodiments, the oral drug dosage form further comprises an erodible restrainer configured to inhibit extension of the moveable arm.

In other aspects, provided herein is an oral drug dosage form configured for a gastric retention, the oral drug dosage form comprising: a swellable material, wherein the swellable material is wrapped, at least in part, with a semi-permeable membrane; a first moveable arm, a second movable arm, a third moveable arm, and a fourth moveable arm, wherein the first moveable arm, the second moveable arm, the third moveable arm, the fourth moveable arm are configured such that each arm rotates on an independent axis in a direction towards a perpendicularly situated plane via a force that is provided by the swellable material; and a body comprising: a first point of connection for the first arm, a second point of connection for the second arm, a third point of connection for the third arm, and a fourth point of connection for the fourth arm, wherein each point of connection is configured to provide the independent axis for rotation of the first arm, the second arm, the third arm, and the fourth arm, wherein each moveable arm comprises a contact element configured to interface with the swellable material or the semi-permeable material, and a first stop, a second stop, a third stop, and a fourth stop configured to engage with the first moveable arm, the second moveable arm, the third moveable arm, and the fourth moveable arm, respectively, at an extended position, wherein the oral drug dosage form is configured to have a pre-administration state having a compact form and a post-administration state having an extended form providing the gastric retention, and wherein the extended form of the post-administration state of the oral drug dosage form is due to, at least in part, expansion of the swellable material in the presence of a gastrointestinal fluid. In some embodiments, the body does not comprise a swellable material compartment. In some embodiments, the swellable material is positioned on the body such that expansion of the swellable material actuates the first moveable arm, the second moveable arm, the third moveable arm, and the fourth moveable arm to an extended position.

In other aspects, provided herein is a swelling structure for use in an oral drug dosage form, the swelling structure comprising: a swellable material; a shell comprising: an outer shell comprising at least two tiers, wherein the at least two tiers have sequentially smaller outer dimensions, wherein the at least two tiers are configured to have a pre-administration state having a compact form wherein at least two of the at least two tiers slide along an axis, wherein the at least two tiers are configured to have a post-administration state having an extended form wherein the at least two tiers extend along the axis, and wherein the post-administration state having the extended form provides the gastric retention, one or more fluid inlets operably connected to the swellable material compartment; and wherein the swelling structure expands due to, at least in part, expansion of the swellable material in the presence of a gastrointestinal fluid. In some embodiments, the at least two tiers of the outer shell are coaxial. In some embodiments, the at least two tiers of the outer shell are concentric.

In other aspects, provided herein is an oral drug dosage form configured for a gastric retention, the oral drug dosage form comprising: an expandable material; a first moveable arm, a second movable arm, a third moveable arm, and a fourth moveable arm, wherein the first moveable arm, the second moveable arm, the third moveable arm, the fourth moveable arm are configured such that each arm rotates on an independent axis in a direction towards a perpendicularly situated plane via a force that is provided by the expandable material, a body comprising: an expandable material compartment configured to contain at least a portion of the expandable material; a directional channel and/or an orifice operably connected to the expandable material compartment, wherein each moveable arm comprises a contact element configured to interface with the expandable material in proximity to the directional channel and/or the orifice, and wherein the expandable material compartment, the contact elements of the moveable arms, and a cap substantially surround the expandable material; a first stop, a second stop, a third stop, and a fourth stop configured to engage with the first moveable arm, second moveable arm, third moveable arm, and the fourth moveable arm, respectively, at an extended position; and a drug, wherein the oral drug dosage form is configured to have a pre-administration state having a compact form and a post-administration state having an extended form providing the gastric retention, and wherein the extended form of the post-administration state of the oral drug dosage form is due to, at least in part, expansion of the expandable material in the presence of a gastrointestinal fluid.

All references cited herein, including patent applications and publications, are incorporated herein by reference in their entirety.

It will also be understood by those skilled in the art that changes in the form and details of the implementations described herein may be made without departing from the scope of this disclosure. In addition, although various advantages, aspects, and objects have been described with reference to various implementations, the scope of this disclosure should not be limited by reference to such advantages, aspects, and objects.

### BRIEF DESCRIPTION OF THE DRAWINGS

FIGS. 1A-1D show diagrams of an example drug dosage form 100 comprising a swellable material 102 and a moveable arm 108, including component views and schematics thereof (FIGS. 1C and 1D).

FIGS. 2A-2D show diagrams of an example drug dosage form 200 comprising a swellable material 202 and a moveable arm 208, including component views and schematics thereof (FIGS. 2C and 2D). FIGS. 2E and 2F show diagrams of an example drug dosage form 250, and component views and schematics are provided in FIG. 2G.

FIGS. 3A and 3B show diagrams of an example drug dosage form 300 comprising a swellable material 302 and a moveable arm 308. Component view schematics are provided in FIG. 3C.

FIGS. 4A-4D show diagrams of an example drug dosage form 400 comprising a swellable material 402 and two described movable arms 408, 418, including component views and schematics thereof (FIGS. 4C and 4D).

FIGS. 5A-5F show diagrams of an example drug dosage form 500 comprising two moveable arms 508, 520 each having a respective swellable material and swellable material compartment, including component views and schematics thereof (FIGS. 5E and 5F).

FIGS. 6A-6H show diagrams of an example drug dosage form 600 comprising four moveable arms 614, 616, 630, 632, including component views and schematics thereof (FIGS. 6E-6H).

FIGS. 7A-7I show diagrams of an example drug dosage form 700 comprising two moveable arms 704, 706 that rotate on an axis 730, including component views and schematics thereof (FIGS. 7G-7I).

FIGS. 8A-8I show diagrams of an example drug dosage form 800 comprising four moveable arms 814, 816, 818, 820 that each rotate on an axis, e.g., 824, including component views and schematics thereof (FIGS. 8E-8I).

FIGS. 9A-9I show diagrams of an example drug dosage form 900 comprising four moveable arms 914, 916, 918, 920 that each rotate on an axis, e.g., 924, including component views and schematics thereof (FIGS. 9E-9I).

FIGS. 10A-10F show diagrams of an example drug dosage form 1000 comprising four moveable arms 1014, 1016, 1018, 1020 that each rotate on an axis, e.g., 1024, including component views thereof (FIGS. 10E and 10F).

FIGS. 11A-11E show diagrams of an example drug dosage form 1100 comprising four moveable arms 1114, 1116, 1118, 1120 that each rotate on an axis, e.g., 1124, including component views and schematics thereof (FIGS. 11D and 11E).

FIGS. 12A-12F show diagrams of an example swelling structure 1200 comprising four tiers 1206, 1208, 1210, and 1212 expand in a post-administration state due to expansion of the swellable material 1202.

FIGS. 13A-13F show diagrams of an example swelling structure 1300 comprising four tiers 1306, 1308, 1310, and 1312 expand in a post-administration state due to expansion of the swellable material 1302.

FIGS. 14A and 14B show diagrams of an example drug dosage form 1400 comprising a swellable material 1410, a column 1412, and four moveable arms.

FIGS. 15A-15D show diagrams of an example drug dosage form 1500 having two pairs of two moveable arms that rotate on a shared axis (a first moveable arm 1504 and a third moveable arm 1516 rotate on a first axis 1512; a second moveable arm 1506 and a fourth moveable arm 1518 rotate on a second axis 1514), including component views thereof (FIG. 15D).

FIG. 16 shows pictures of models of oral drug dosage forms following expansion of swellable materials.

FIG. 17 shows pictures of partial models of oral drug dosage forms following expansion of swellable materials.

FIG. 18 shows pictures of replicate models of an oral drug dosage form and measurements of moveable arm expansion.

FIGS. 19A and 19B shows X-ray images of an oral drug dosage form following administration and in vivo status information of the oral drug dosage form.

FIGS. 20A and 20B shows X-ray images of an oral drug dosage form following administration and in vivo status information of the oral drug dosage form.

FIGS. 21A-21C show diagrams of an example drug dosage form 2100 comprising four moveable arms 2114, 2116, 2118, 2120 that each rotate on an axis, e.g., 2124.

FIGS. 22A-22B show diagrams of an example drug dosage form 2200 comprising two moveable arms 2214, 2216 that each rotate on an axis, e.g., 2226.

FIGS. 23A and 23B shows pre and post administration of the drug dosage form in vitro status.

FIG. 24 provides information obtained from gastroscopy examination of beagle dogs administered certain oral drug dosage forms.

FIG. 25 provides results from Occult Blood tests of beagle dogs administered certain oral drug dosage forms.

FIG. 26 provides residues of certain oral drug dosage forms collected from feces of beagle dogs administered certain oral drug dosage forms.

### DETAILED DESCRIPTION

Provided herein, in some aspects, are oral drug dosage forms comprising a swellable material configured, upon swelling, to drive expansion of the overall size of the oral drug dosage forms such that the oral drug dosage forms are retained in the stomach for a desired period of time. The oral drug dosage forms provided herein are based, at least in part, on the inventors' unique perspectives and findings regarding oral drug dosage form designs and mechanisms that provide oral drug dosage forms small enough for convenient patient administration and have a sufficient size increase in the stomach to inhibit passage through the pylorus for a desired time (i.e., the oral drug dosage forms are retained in the stomach for an extended time, e.g., between 8 hours and 3 months). In certain aspects, the oral drug dosage forms of the present application comprise one or more movable arms actuated by way of swelling of a swellable material, wherein after increasing in size the oral drug dosage form retains sufficient mechanical stability to enable stomach retention for extended periods of time without interfering with normal stomach function. The drug dosage forms of the present application can also be configured to release a drug therefrom following any desired drug release profile of one or more drugs (e.g., an immediate-release profile, a sustained-release profile, a delayed-sustained release profile, a pulsed release profile, or any combination thereof).

Thus, in some aspects, provided is an oral drug dosage form configured for gastric retention, the oral drug dosage form comprising: a swellable material; a moveable arm configured such that at least a portion of the moveable arm is extendible beyond, or further away from, a body of the oral drug dosage form via a force that is provided by the swellable material; and the body comprising: a swellable material compartment configured to contain at least a portion of the swellable material; a directional channel and/or an orifice operably connected to the swellable material compartment, wherein the moveable arm comprises a contact element configured to interface with the swellable material, or a feature associated with the swellable material, in proximity to the directional channel and/or the orifice; and one or more fluid inlets operably connected to the swellable material compartment; and a drug, wherein the oral drug dosage form is configured to have a pre-administration state having a compact form and a post-administration state having an extended form providing the gastric retention, and wherein the extended form of the post-administration state of the oral drug dosage form is due to, at least in part, expansion of the swellable material in the presence of a gastrointestinal fluid.

In other aspects, provided herein is an oral drug dosage form configured for a gastric retention, the oral drug dosage form comprising: a swellable material; a moveable arm configured such that at least a portion of the moveable arm is extendible beyond, or further away from, a body of the oral drug dosage form via a force that is provided by the swellable material; the body comprising: a swellable material compartment configured to contain at least a portion of the swellable material; a directional channel operably connected to the swellable material compartment, wherein the moveable arm comprises a contact element configured to interface with the swellable material in proximity to the directional channel, wherein the swellable material compartment and the contact element of the moveable arm substantially surround the swellable material, and wherein the moveable arm, or a portion thereof, is configured to slide within the directional channel such that the moveable arm extends beyond, or further away from, the body of the oral drug dosage form along an axis based on the directional channel; one or more fluid inlets operably connected to the swellable material compartment; a stop configured to engage with the moveable arm at an extended position; and a drug, wherein the oral drug dosage form is configured to have a pre-administration state having a compact form and a post-administration state having an extended form providing the gastric retention, and wherein the extended form of the post-administration state of the oral drug dosage form is due to, at least in part, expansion of the swellable material in the presence of a gastrointestinal fluid.

In other aspects, provided is an oral drug dosage form configured for a gastric retention, the oral drug dosage form comprising: a swellable material; a first moveable arm and a second movable arm, wherein the first moveable arm and the second moveable arm are configured such that at least a portion of each moveable arm is extendible beyond, or further away from, a body of the oral drug dosage form via a force that is provided by the swellable material, and wherein the first moveable arm and the second moveable arm extend in substantially opposite directions along an axis based on the directional channel; the body comprising: a swellable material compartment configured to contain at least a portion of the swellable material; a first directional channel and a second directional channel operably connected to the swellable material compartment, wherein the first moveable arm comprises a first contact element configured to interface with the swellable material in proximity to the first directional channel, wherein the second moveable arm comprises a second contact element configured to interface with the swellable material in proximity to the second directional channel, wherein the swellable material compartment and the first contact element of the first moveable arm and second contact element of the second moveable arm substantially surround the swellable material, and wherein the first moveable arm, or a portion thereof, is configured to slide within the first directional channel such that the first moveable arm extends beyond, or further away from, the body of the oral drug dosage form along the axis, and wherein the second moveable arm, or a portion thereof, is configured to slide within the second directional channel such that the second moveable arm extends beyond, or further away from, the body of the oral drug dosage form along the axis; one or more fluid inlets operably connected to the swellable material compartment; a first stop configured to engage with the first moveable arm at an extended position; a second stop configured to engage with the second moveable arm at an extended position; and a drug, wherein the oral drug dosage form is configured to have a pre-administration state having a compact form and a post-administration state having an extended form providing the gastric retention, and wherein the extended form of the post-administration state of the oral drug dosage form is due to, at least in part, expansion of the swellable material in the presence of a gastrointestinal fluid.

In other aspects, provided herein is an oral drug dosage form configured for a gastric retention, the oral drug dosage form comprising: a first swellable material; a first moveable arm, wherein the first moveable arm is configured such that at least a portion of the moveable arm is extendible beyond, or further away from, a body of the oral drug dosage form via a force that is provided by the first swellable material, and wherein the first moveable arm is configured to rotate on a first axis; a second swellable material; a second moveable arm, wherein the second moveable arm is configured such that at least a portion of the moveable arm is extendible beyond, or further away from, the body of the oral drug dosage form via a force that is provided by the second swellable material, and wherein the second moveable arm is configured to rotate on a second axis; the body comprising: a first swellable material compartment configured to contain at least a portion of the first swellable material; a first directional channel operably connected to the first swellable material compartment, wherein the first direction channel comprises a curved-shaped channel, wherein the first moveable arm comprises a first contact element configured to interface with the first swellable material in proximity to the first directional channel, wherein the first swellable material compartment and the first contact element of the first moveable arm substantially surround the first swellable material, and wherein the first moveable arm, or a portion thereof, is configured to rotate on the first axis by way of the first contact element sliding in the first direction channel such that the first moveable arm extends beyond, or further away from, the body of the oral drug dosage form, one or more fluid inlets operably connected to the first swellable material compartment; a second swellable material compartment configured to contain at least a portion of the second swellable material; a second directional channel operably connected to the second swellable material compartment, wherein the second direction channel comprises a curved-shaped channel, wherein the second moveable arm comprises a second contact element configured to interface with the second swellable material in proximity to the second directional channel, wherein the second swellable material compartment and the second contact element of the second moveable arm substantially surround the second swellable material, and wherein the second moveable arm, or a portion thereof, is configured to rotate on the second axis by way of the second contact element sliding in the second direction channel such that the second moveable arm extends beyond, or further away from, the body of the oral drug dosage form, one or more fluid inlets operably connected to the second swellable material compartment; a drug, wherein the oral drug dosage form is configured to have a pre-administration state having a compact form and a post-administration state having an extended form providing the gastric retention, and wherein the extended form of the post-administration state of the oral drug dosage form is due to, at least in part, expansion of the swellable material in the presence of a gastrointestinal fluid.

In other aspects, provided herein is an oral drug dosage form configured for a gastric retention, the oral drug dosage form comprising: a first swellable material; a first moveable arm and a second movable arm, wherein the first moveable arm and the second moveable arm are configured such that at least a portion of each moveable arm is extendible beyond, or further away from, a body of the oral drug dosage form via a force that is provided by the first swellable material, and wherein the first moveable arm and the second moveable arm extend in substantially opposite directions along an axis based on the directional channel; a second swellable material; a third moveable arm, wherein the third moveable arm is configured such that at least a portion of the third moveable arm is extendible beyond, or further away from, the body of the oral drug dosage form via a force that is provided by the second swellable material, and wherein the third moveable arm is configured to rotate on a first axis; a third swellable material; a fourth moveable arm, wherein the fourth moveable arm is configured such that at least a portion of the fourth moveable arm is extendible beyond, or further away from, the body of the oral drug dosage form via a force that is provided by the third swellable material, and wherein the fourth moveable arm is configured to rotate on a second axis; the body comprising: a first swellable material compartment configured to contain at least a portion of the first swellable material; a first directional channel and a second directional channel operably connected to the first swellable material compartment, wherein the first moveable arm comprises a first contact element configured to interface with the first swellable material in proximity to the first directional channel, wherein the second moveable arm comprises a second contact element configured to interface with the first swellable material in proximity to the second directional channel, wherein the first swellable material compartment and the first contact element of the first moveable arm and second contact element of the second moveable arm substantially surround the first swellable material, and wherein the first moveable arm, or a portion thereof, is configured to slide within the first directional channel such that the first moveable arm extends beyond, or further away from, the body of the oral drug dosage form along the axis, and wherein the second moveable arm, or a portion thereof, is configured to slide within the second directional channel such that the second moveable arm extends beyond, or further away from, the body of the oral drug dosage form along the axis; one or more fluid inlets operably connected to the first swellable material compartment; a first stop configured to engage with the first moveable arm at an extended position; a second stop configured to engage with the second moveable arm at an extended position; a second swellable material compartment configured to contain at least a portion of the second swellable material; a third directional channel operably connected to the second swellable material compartment, wherein the third direction channel comprises a curved-shaped channel, wherein the third moveable arm comprises a contact element configured to interface with the second swellable material in proximity to the third directional channel, wherein the second swellable material compartment and the contact element of the third moveable arm substantially surround the second swellable material, and wherein the third moveable arm, or a portion thereof, is configured to rotate on the first axis by way of the contact element sliding in the third direction channel such that the third moveable arm extends beyond, or further away from, the body of the oral drug dosage form, one or more fluid inlets operably connected to the third swellable material compartment; a third swellable material compartment configured to contain at least a portion of the third swellable material; a fourth directional channel operably connected to the third swellable material compartment, wherein the fourth direction channel comprises a curved-shaped channel, wherein the fourth moveable arm comprises a contact element configured to interface with the third swellable material in proximity to the fourth directional channel, wherein the third swellable material compartment and the contact element of the fourth moveable arm substantially surround the third swellable material, and wherein the fourth moveable arm, or a portion thereof, is configured to rotate on the second axis by way of the contact element sliding in the fourth direction channel such that the fourth moveable arm extends beyond, or further away from, the body of the oral drug dosage form, one or more fluid inlets operably connected to the third swellable material compartment; and a drug, wherein the oral drug dosage form is configured to have a pre-administration state having a compact form and a post-administration state having an extended form providing the gastric retention, and wherein the extended form of the post-administration state of the oral drug dosage form is due to, at least in part, expansion of the first swellable material, the second swellable material, and the third swellable material in the presence of a gastrointestinal fluid.

In other aspects, provided herein is an oral drug dosage form configured for a gastric retention, the oral drug dosage form comprising: a swellable material; a first moveable arm and a second movable arm, wherein the first moveable arm and the second moveable arm are configured such that at least a portion of each moveable arm is extendible beyond, or further away from, a body of the oral drug dosage form via a force that is provided by the swellable material, and wherein the first moveable arm and the second moveable arm are configured to rotate on a shared axis in opposite directions; the body comprising: a swellable material compartment configured to contain at least a portion of the swellable material; a first directional channel and a second directional channel operably connected to the swellable material compartment, wherein the first directional channel and the second directional channel are curved-shaped and configured around the shared axis, wherein the first moveable arm comprises a first contact element configured to interface with the swellable material in proximity to the first directional channel, wherein the first contact element is rudder-shaped and configured to travel in the first directional channel such that the first moveable arm rotates beyond, or further away from, the body of the oral drug dosage form relative to the shared axis, wherein the second moveable arm comprises a second contact element configured to interface with the swellable material in proximity to the second directional channel, wherein the second contact element is rudder-shaped and configured to travel in the second directional channel such that the second moveable arm rotates beyond, or further away from, the body of the oral drug dosage form relative to the shared axis, one or more fluid inlets operably connected to the swellable material compartment; a stop configured to engage with the first moveable arm and the second moveable arm at an extended position; a drug, wherein the oral drug dosage form is configured to have a pre-administration state having a compact form and a post-administration state having an extended form providing the gastric retention, and wherein the extended form of the post-administration state of the oral drug dosage form is due to, at least in part, expansion of the swellable material in the presence of a gastrointestinal fluid.

In other aspects, provided herein is an oral drug dosage form configured for a gastric retention, the oral drug dosage form comprising: a swellable material; a first moveable arm, a second movable arm, a third moveable arm, and a fourth moveable arm, wherein the first moveable arm, the second moveable arm, the third moveable arm, the fourth moveable arm are configured such that each arm rotates on an independent axis in a direction towards a perpendicularly situated plane via a force that is provided by the swellable material, a body comprising: a swellable material compartment configured to contain at least a portion of the swellable material; a directional channel and/or an orifice operably connected to the swellable material compartment, wherein each moveable arm comprises a contact element configured to interface with the swellable material in proximity to the directional channel and/or the orifice, and wherein the swellable material compartment, the contact elements of the moveable arms, and a cap substantially surround the swellable material; one or more fluid inlets operably connected to the swellable material compartment; a cap; a first stop, a second stop, a third stop, and a fourth stop configured to engage with the first moveable arm, second moveable arm, third moveable arm, and the fourth moveable arm, respectively, at an extended position; and a drug, wherein the oral drug dosage form is configured to have a pre-administration state having a compact form and a post-administration state having an extended form providing the gastric retention, and wherein the extended form of the post-administration state of the oral drug dosage form is due to, at least in part, expansion of the swellable material in the presence of a gastrointestinal fluid.

In other aspects, provided herein is an oral drug dosage form configured for a gastric retention, the oral drug dosage form comprising: a swellable material; a first moveable arm, a second movable arm, a third moveable arm, and a fourth moveable arm, wherein the first moveable arm, the second moveable arm, the third moveable arm, the fourth moveable arm are configured such that each arm rotates on an independent axis in a direction towards a perpendicularly situated plane via a force that is provided by the swellable material, a body comprising: a swellable material compartment configured to contain at least a portion of the swellable material; a directional channel and/or an orifice operably connected to the swellable material compartment, wherein each moveable arm comprises a contact element configured to interface with the swellable material in proximity to the directional channel and/or the orifice, and wherein the swellable material compartment and the contact elements of the moveable arms substantially surround the swellable material; one or more fluid inlets operably connected to the swellable material compartment; a first stop, a second stop, a third stop, and a fourth stop configured to engage with the first moveable arm, second moveable arm, third moveable arm, and the fourth moveable arm, respectively, at an extended position; and a drug, wherein the oral drug dosage form is configured to have a pre-administration state having a compact form and a post-administration state having an extended form providing the gastric retention, and wherein the extended form of the post-administration state of the oral drug dosage form is due to, at least in part, expansion of the swellable material in the presence of a gastrointestinal fluid.

In other aspects, provided herein is an oral drug dosage form configured for a gastric retention, the oral drug dosage form comprising: a swellable material, wherein the swellable material is wrapped, at least in part, with a semi-permeable membrane; a first moveable arm, a second movable arm, a third moveable arm, and a fourth moveable arm, wherein the first moveable arm, the second moveable arm, the third moveable arm, the fourth moveable arm are configured such that each arm rotates on an independent axis in a direction towards a perpendicularly situated plane via a force that is provided by the swellable material; and a body comprising: a first point of connection for the first arm, a second point of connection for the second arm, a third point of connection for the third arm, and a fourth point of connection for the fourth arm, wherein each point of connection is configured to provide the independent axis for rotation of the first arm, the second arm, the third arm, and the fourth arm, wherein each moveable arm comprises a contact element configured to interface with the swellable material or the semi-permeable material, and a first stop, a second stop, a third stop, and a fourth stop configured to engage with the first moveable arm, the second moveable arm, the third moveable arm, and the fourth moveable arm, respectively, at an extended position, wherein the oral drug dosage form is configured to have a pre-administration state having a compact form and a post-administration state having an extended form providing the gastric retention, and wherein the extended form of the post-administration state of the oral drug dosage form is due to, at least in part, expansion of the swellable material in the presence of a gastrointestinal fluid.

In other aspects, provided herein is a swelling structure for use in an oral drug dosage form, the swelling structure comprising: a swellable material; a shell comprising: an outer shell comprising at least two tiers, wherein the at least two tiers have sequentially smaller outer dimensions, wherein the at least two tiers are configured to have a pre-administration state having a compact form wherein at least two of the at least two tiers slide along an axis, wherein the at least two tiers are configured to have a post-administration state having an extended form wherein the at least two tiers extend along the axis, and wherein the post-administration state having the extended form provides the gastric retention, one or more fluid inlets operably connected to the swellable material compartment; and wherein the swelling structure expands due to, at least in part, expansion of the swellable material in the presence of a gastrointestinal fluid.

In other aspects, provided herein is an oral drug dosage form configured for a gastric retention, the oral drug dosage form comprising: an expandable material; a first moveable arm, a second movable arm, a third moveable arm, and a fourth moveable arm, wherein the first moveable arm, the second moveable arm, the third moveable arm, the fourth moveable arm are configured such that each arm rotates on an independent axis in a direction towards a perpendicularly situated plane via a force that is provided by the expandable material, a body comprising: an expandable material compartment configured to contain at least a portion of the expandable material; a directional channel and/or an orifice operably connected to the expandable material compartment, wherein each moveable arm comprises a contact element configured to interface with the expandable material in proximity to the directional channel and/or the orifice, and wherein the expandable material compartment, the contact elements of the moveable arms, and a cap substantially surround the expandable material; a first stop, a second stop, a third stop, and a fourth stop configured to engage with the first moveable arm, second moveable arm, third moveable arm, and the fourth moveable arm, respectively, at an extended position; and a drug, wherein the oral drug dosage form is configured to have a pre-administration state having a compact form and a post-administration state having an extended form providing the gastric retention, and wherein the extended form of the post-administration state of the oral drug dosage form is due to, at least in part, expansion of the expandable material in the presence of a gastrointestinal fluid.

### I. Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

As used herein, the term “individual” refers to a mammal and includes, but is not limited to, human, bovine, horse, feline, canine, rodent, rat, mouse, dog, or primate. In some embodiments, the individual is a human individual.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “an,” “or,” and “the” include plural referents unless the context clearly dictates otherwise.

### II. Oral Drug Dosage Forms Configured for Gastric Retention

The present disclosure, in some aspects, is directed to drug dosage forms designed to provide a desired retention in an individual based on geometric differences between a pre-administration and post-administration state of the dosage form, e.g., a change in size of one or more dimensions. In some aspects of the description provided herein, oral drug dosage forms are exemplified, however, it is to be understood that the teachings of such dosage forms can be readily extrapolated to other drug dosage forms, such as dosage form suitable for vaginal or rectal administration. In certain aspects, provided herein are oral drug dosage forms configured for gastric retention, and components thereof. The oral drug dosage forms provided herein are configured with swellable material, including one or more portions of swellable material being the composed of the same or different material, such that the swellable material upon exposure to gastrointestinal fluid swells and provides a force to actuate a mechanism to increase the size of the oral drug dosage form. This increase in the size of the oral drug dosage form leads to the oral drug dosage form being retained in the stomach for an extended time as it cannot readily pass through they pylorus.

In some embodiments, the drug dosage form, or a component thereof, undergoes expansion due to a bodily fluid. For example, in some embodiments, the bodily fluid is a gastrointestinal fluid.

In some embodiments, provided is an oral drug dosage form configured for gastric retention, the oral drug dosage form comprising: a swellable material; a moveable arm configured such that at least a portion of the moveable arm is extendible beyond, or further away from, a body of the oral drug dosage form via a force that is provided by the swellable material; and the body comprising: a swellable material compartment configured to contain at least a portion of the swellable material; a directional channel and/or an orifice operably connected to the swellable material compartment, wherein the moveable arm comprises a contact element configured to receive the force generated by the swellable material (such as configured to interface with the swellable material, or a feature associated with the swellable material) in proximity to the directional channel and/or the orifice; and one or more fluid inlets operably connected to the swellable material compartment; and a drug, wherein the oral drug dosage form is configured to have a pre-administration state having a compact form and a post-administration state having an extended form providing the gastric retention, and wherein the extended form of the post-administration state of the oral drug dosage form is due to, at least in part, expansion of the swellable material in the presence of a gastrointestinal fluid. In some embodiments, the drug is located in one or more of: one or more moveable arms, or the body (or a component thereof, such as a cap).

In some embodiments, the swellable material is a chemical composition; In some embodiments, the swellable material compartment comprises a chemical composition and a plunger structure, wherein the plunger structure has a certain rigidity, and the swollen composition pushes it to move towards the orifice, thereby pushing the movable arm to unfold; In some embodiments, the piston is pushed beyond the orifice; In some embodiments, the piston moves in the direction of the orifice until it is blocked by a cap; In some embodiments, the plunger structure is a piston, which has a stronger rigidity than the swelling composition.

In some embodiments, provided is an oral drug dosage form configured for a gastric retention, the oral drug dosage form comprising: a swellable material, wherein the swellable material is wrapped, at least in part, with a semi-permeable membrane; In some embodiments, the body or base is all semi-permeable membrane; a first moveable arm, a second movable arm, a third moveable arm, and a fourth moveable arm, wherein the first moveable arm, the second moveable arm, the third moveable arm, the fourth moveable arm are configured such that each arm rotates on an independent axis in a direction towards a perpendicularly situated plane via a force that is provided by the swellable material; and a body comprising: a first point of connection for the first arm, a second point of connection for the second arm, a third point of connection for the third arm, and a fourth point of connection for the fourth arm, wherein each point of connection is configured to provide the independent axis for rotation of the first arm, the second arm, the third arm, and the fourth arm, wherein each moveable arm comprises a contact element configured to interface with the swellable material or the semi-permeable material, and a first stop, a second stop, a third stop, and a fourth stop configured to engage with the first moveable arm, second moveable arm, third moveable arm, and the fourth moveable arm, respectively, at an extended position, wherein the oral drug dosage form is configured to have a pre-administration state having a compact form and a post-administration state having an extended form providing the gastric retention, and wherein the extended form of the post-administration state of the oral drug dosage form is due to, at least in part, expansion of the swellable material in the presence of a gastrointestinal fluid.

As described herein, the oral drug dosage forms comprise moving parts, such as involved with the transitions between a pre-administration state (e.g., compact for purposes of oral administration) and a post-administration state (e.g., extended for purposes of gastric retention). Accordingly, there may be certain variations between the most compact and the most extended states reached by oral drug dosage forms during use. Description of a single pre-administration state and a single post-administration state is not intended to limit the scope of the description provided herein, and as taught herein there may be more than one pre-administration state and/or more than one post-administration state experienced by an oral drug dosage form during an administration life cycle. Additionally, the oral drug dosage forms provided herein are configured to eventually clear from the stomach of the individual to whom the oral drug dosage form was administered.

Additional sections are provided below to teach example mechanisms involved with providing gastric retention to oral drug dosage forms, and certain aspects of the oral drug dosage forms. Such modular description is not intended to limit the scope of the description provided herein, and based on the teaching provided herein one of ordinary skill in the art will readily appreciate that the description encompasses any combination of modules brought together to form an oral drug dosage form having gastric retention.

a. Example Oral Drug Dosage Forms

For purposes of illustration and explanation of the subject matter provided herein, certain drug dosage forms are described below, such as oral drug dosage forms. As described, the fundamental teachings described herein enable various moveable arm mechanisms to provide a size increase of an oral drug dosage form such that the oral drug dosage form does not readily pass through the pylorus for a desired period of time and thus exhibits a period of gastric retention. In certain aspects, the oral drug dosage form taught herein includes a single taught mechanism for post-administration size increase. In some embodiments, two or more instances of the single mechanism, such as a sliding arm, may be used in a single oral drug dosage form. In certain aspects, an oral drug dosage form taught herein includes more than one taught mechanism for post-administration size increase, such as the use of two styles of moveable arms.

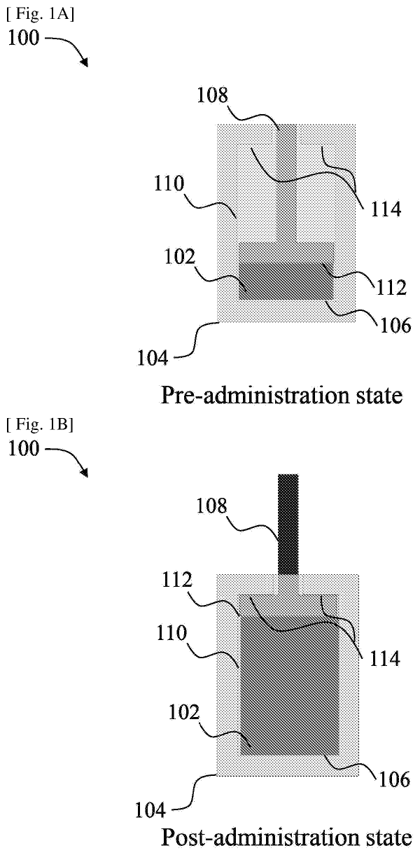

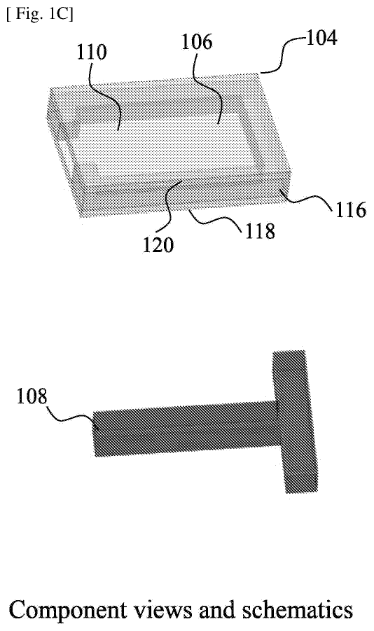

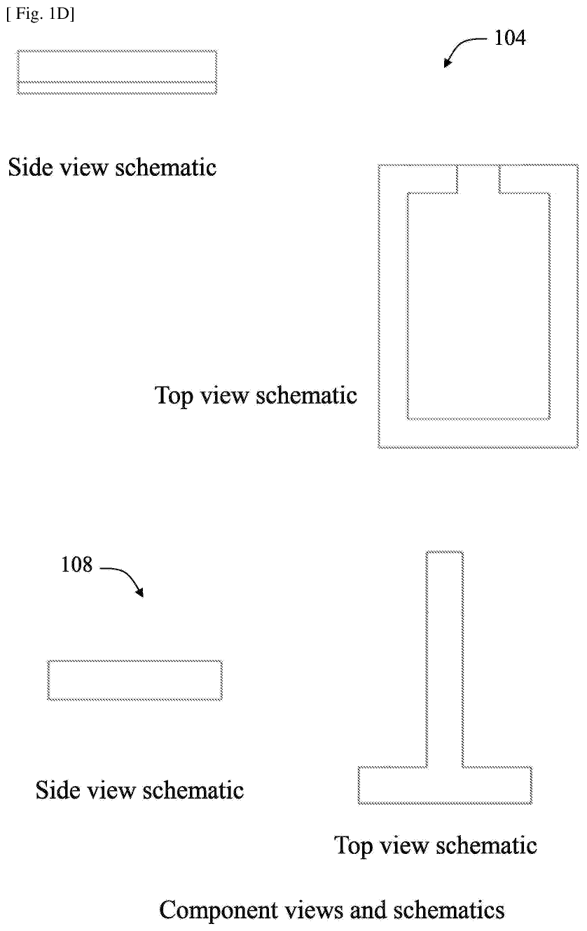

An example oral drug dosage form 100 configured for gastric retention is provided in FIGS. 1A-1C. As shown in a pre-administration state in FIG. 1A, the oral drug dosage form 100 comprises a swellable material 102 contained in a swellable material compartment 106 formed by a body 104 of the oral drug dosage form 100. The oral drug dosage form 100 comprises a moveable arm 108 positioned within (or substantially within) the footprint of the oral drug dosage form 100 in the pre-administration state. The moveable arm 108 is configured such that at least a portion of the moveable arm is extendible beyond, or further away from, the body 104 of the oral drug dosage form 100 via a force that is provided by the swellable material 102. The body 104 of the oral drug dosage form 100 forms a directional channel 110 configured to guide the movement of the moveable arm 108 upon swelling of the swellable material 102. In the pre-administration state, the oral drug dosage form 100 is designed such that the moveable arm 108 comprises a contact element 112 configured to interface with the swellable material 102 in proximity to the directional channel 110. As the swellable material 102 swells, the swellable material 102 will push against the contact element 112 of the moveable arm 108 to move the moveable arm 108. The body 104 also forms a stop 114 configured to engage with the moveable arm 108 at an extended position. A post-administration state of the oral drug dosage form 100 is shown in FIG. 1B. In the post-administration state, gastrointestinal fluids have entered the oral drug dosage form 100 such that the swellable material 102 swells and pushes the moveable arm 108 to the stop 114 via the directional channel 110. Component views of the oral drug dosage form 100 are provided in FIG. 1C. As shown in FIG. 1C, the body 104 of the oral drug dosage form 100 may comprise a frame 116 and caps 118, 120. In certain embodiments, the frame 116, or portions thereof, comprise a semi-permeable material that allows gastrointestinal fluid to contact the swellable material. In some embodiments, the semi-permeable material provides gastrointestinal fluid access to the swellable material compartment 106. In some embodiments, the semi-permeable material provides gastrointestinal fluid access to the direction channel 110 such that the swellable material can continue to absorb gastrointestinal fluid during swelling. In some embodiments, the oral drug dosage form may comprise one or more fluid inlets, wherein at least one of the fluid inlets is a pore. Component views of the oral drug dosage form 100 are provided in FIG. 1D. As shown in FIG. 1C, certain oral drug dosage forms may have a seamless transition between a swellable material compartment and a directional channel, wherein a boundary can be assigned, for descriptive purposes, that is consistent with the teaching provided herein. In some embodiments, the components of the oral drug dosage form 100 may be produced, such as via three-dimensional printing, injection molding, ultrasonic welding, or any combination thereof, and then assembled to form the pre-administration oral drug dosage form 100. For example, in some embodiments, the body (or one or more portions thereof) is produced independently from the moveable arm, and such components are later assembled to form the oral drug dosage form.

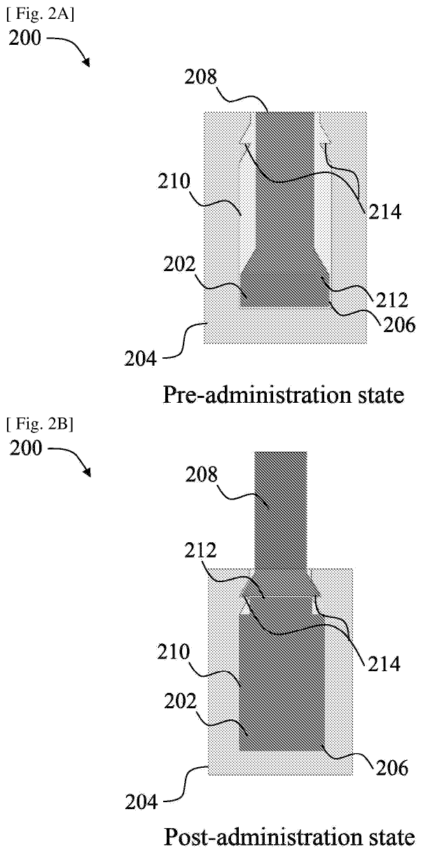

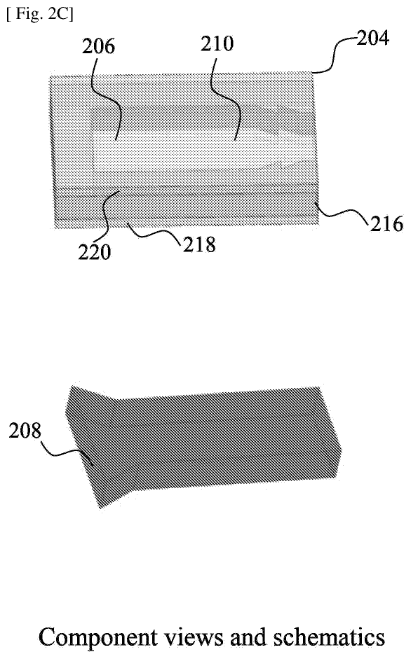

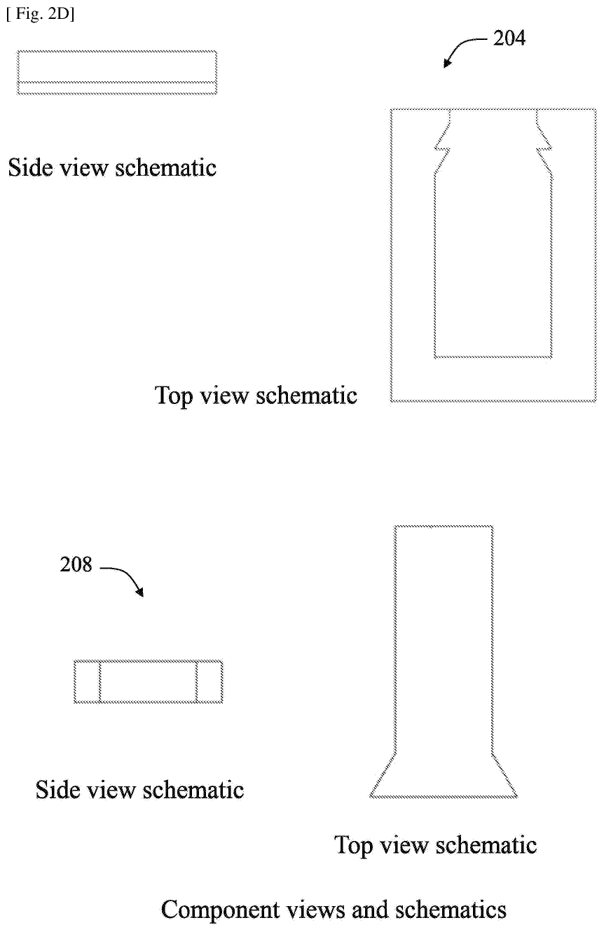

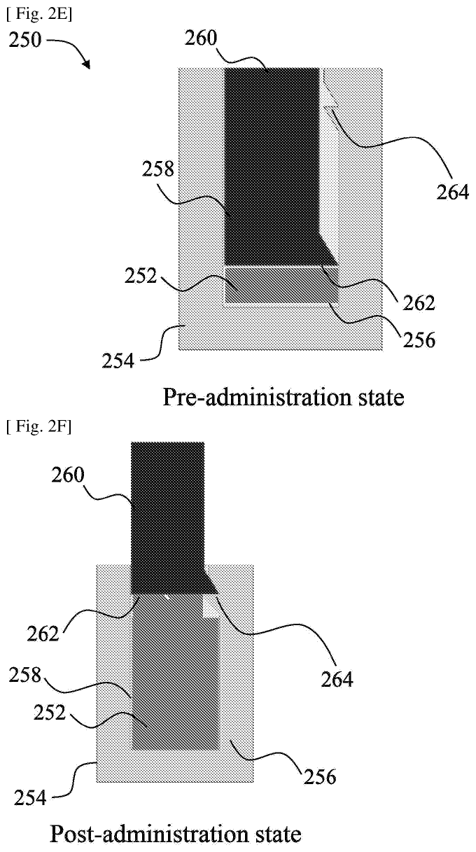

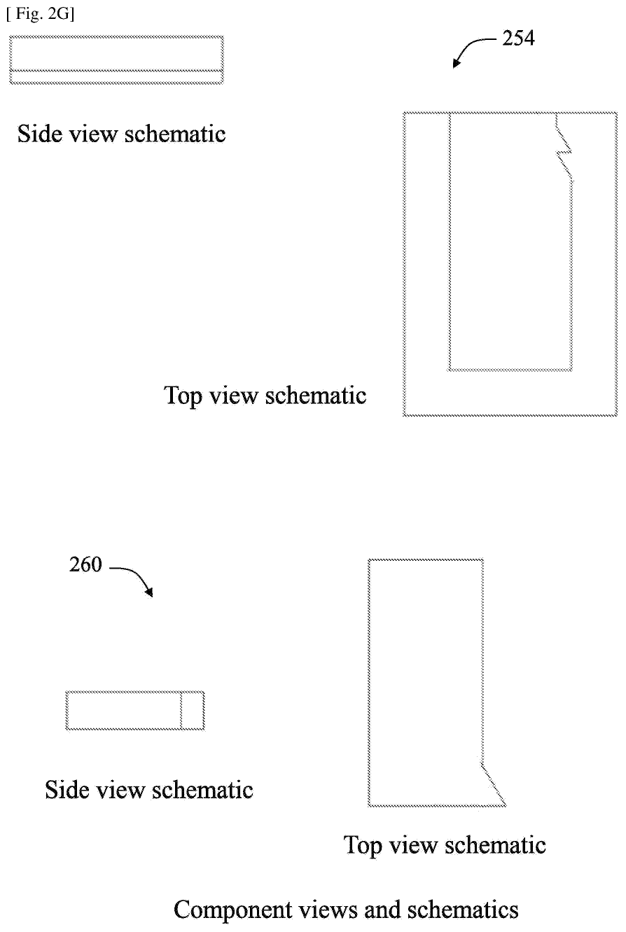

An example oral drug dosage form 200 configured for gastric retention is provided in FIGS. 2A-2C. As shown in a pre-administration state in FIG. 2A, the oral drug dosage form 200 comprises a swellable material 202 contained in a swellable material compartment 206 formed by a body 204 of the oral drug dosage form 200. The oral drug dosage form 200 comprises a moveable arm 208 positioned within (or substantially within) the footprint of the oral drug dosage form 200 in the pre-administration state. The moveable arm 208 is configured such that at least a portion of the moveable arm is extendible beyond, or further away from, the body 204 of the oral drug dosage form 200 via a force that is provided by the swellable material 202. The body 204 of the oral drug dosage form 200 forms a directional channel 210 configured to guide the movement of the moveable arm 208 upon swelling of the swellable material 202. In the pre-administration state, the oral drug dosage form 200 is designed such that the moveable arm 208 comprises a contact element 212 configured to interface with the swellable material 202 in proximity to the directional channel 210. As the swellable material 202 swells, the swellable material 202 will push against the contact element 212 of the moveable arm 208 to move the moveable arm 208. The body 204 also forms a stop 214 comprising a locking element configured to maintain the moveable arm 208 at an extended position in the post-administration state. A post-administration state of the oral drug dosage form 200 is shown in FIG. 2B. In the post-administration state, gastrointestinal fluids have entered the oral drug dosage form 200 such that the swellable material 202 swells and pushes the moveable arm 208 to the stop 214 comprising the locking element via the directional channel 210. As illustrated in FIG. 2B, a portion of the moveable arm 208 engages with the locking element such that the moveable arm 208 is maintained at an extended position (substantial movement in the direction of or opposite to the movement of the moveable arm in the directional channel is inhibited). Component views of the oral drug dosage form 200 are provided in FIG. 2C. As shown in FIG. 2C, the body 204 of the oral drug dosage form 200 may comprise a frame 216 and caps 218, 220. In certain embodiments, the frame 216, or portions thereof, comprise a semi-permeable material that allows gastrointestinal fluid to contact the swellable material. In some embodiments, the semi-permeable material provides gastrointestinal fluid access to the swellable material compartment 206. In some embodiments, the semi-permeable material provides gastrointestinal fluid access to the direction channel 210 such that the swellable material can continue to absorb gastrointestinal fluid during swelling. In some embodiments, the oral drug dosage form may comprise one or more fluid inlets, wherein at least one of the fluid inlets is a pore. Component views of the body 204 and the moveable arm 208 are provided in FIG. 2D. As shown in FIG. 2C, certain oral drug dosage forms may have a seamless transition between a swellable material compartment and a directional channel, wherein a boundary can be assigned, for descriptive purposes, that is consistent with the teaching provided herein. In some embodiments, the components of the oral drug dosage form 200 may be produced, such as via three-dimensional printing, injection molding, ultrasonic welding, or any combination thereof, and then assembled to form the pre-administration oral drug dosage form 200. For example, in some embodiments, the body (or one or more portions thereof) is produced independently from the moveable arm, and such components are later assembled to form the oral drug dosage form.

As shown in FIGS. 2E and 2F, variant designs of oral drug dosage form 200 are provided. Specifically, the oral drug dosage form 250 provided in FIGS. 2E and 2F has a different locking element 264 wherein the locking element 264 engages with a single side of the moveable arm 260. The body 254 of the oral drug dosage form 250 forms a swellable material compartment 256, a direction channel 258 and a stop comprising a locking element 264. The swellable material 252 is contained in the swellable material compartment 256. The moveable arm 260 is configured with a contact surface 262. In the post-administration state FIG. 2F, the swellable material 252 has pushed the moveable arm 260 via the contact surface 262 to an extended position such that the moveable arm 260 engages with the locking element 264 of the stop of the oral drug dosage form 250. Component views of the body 254 and the moveable arm 260 are provided in FIG. 2G.

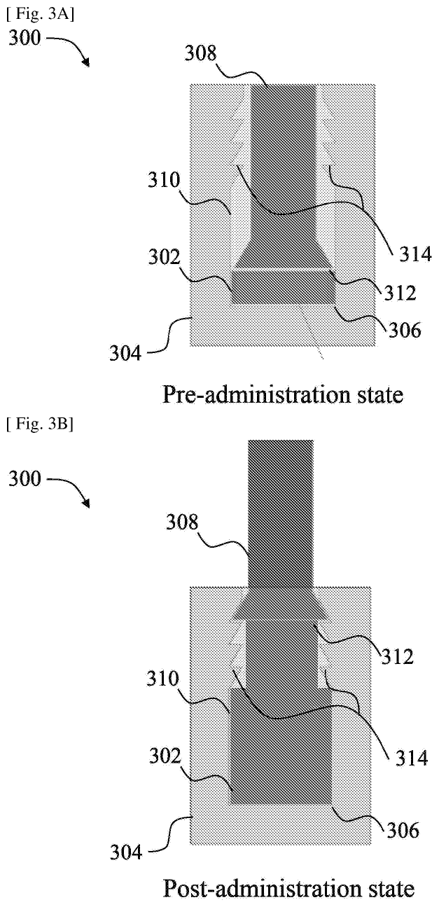

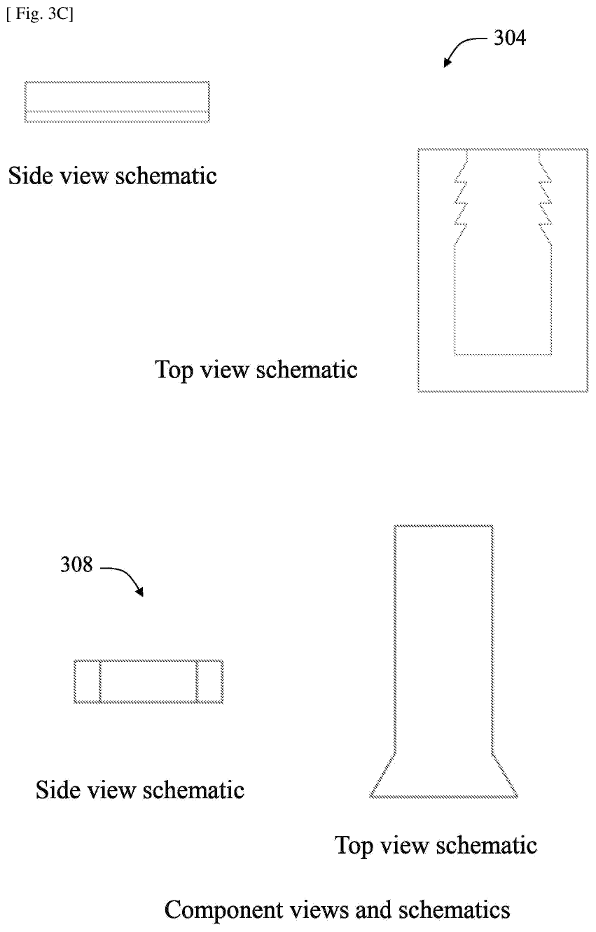

An example oral drug dosage form 300 configured for gastric retention is provided in FIGS. 3A and 3B. As shown in a pre-administration state in FIG. 3A, the oral drug dosage form 300 comprises a swellable material 302 contained in a swellable material compartment 306 formed by a body 304 of the oral drug dosage form 300. The oral drug dosage form 300 comprises a moveable arm 308 positioned within (or substantially within) the footprint of the oral drug dosage form 300 in the pre-administration state. The moveable arm 308 is configured such that at least a portion of the moveable arm is extendible beyond, or further away from, the body 304 of the oral drug dosage form 300 via a force that is provided by the swellable material 302. The body 304 of the oral drug dosage form 300 forms a directional channel 310 configured to guide the movement of the moveable arm 308 upon swelling of the swellable material 302. In the pre-administration state, the oral drug dosage form 300 is designed such that the moveable arm 308 comprises a contact element 312 configured to interface with the swellable material 302 in proximity to the directional channel 310. As the swellable material 302 swells, the swellable material 302 will push against the contact element 312 of the moveable arm 308 to move the moveable arm 308. The body 304 also forms a stop 314 comprising a locking element configured to maintain the moveable arm 308 at an extended position in the post-administration state. A post-administration state of the oral drug dosage form 300 is shown in FIG. 3B. In the post-administration state, gastrointestinal fluids have entered the oral drug dosage form 300 such that the swellable material 302 swells and pushes the moveable arm 308 to the stop 314 comprising the locking element via the directional channel 310. As illustrated in FIG. 3B, the locking element comprises several steps which can engage with the movable arm at various extended positions In some embodiments, the post-administration state may be any one or more of the extended position provided by the several steps of the locking element of the stop. As shown in FIG. 3B, a portion of the moveable arm 308 engages with the locking element such that the moveable arm 308 is maintained at an extended position (substantial movement in the direction of or opposite to the movement of the moveable arm in the directional channel is inhibited; however, it is noted that for steps prior to the final, most-extended step of the locking element that movement is permitted to allow for further extension). Component views of the oral drug dosage form 300 are provided in FIG. 3C.

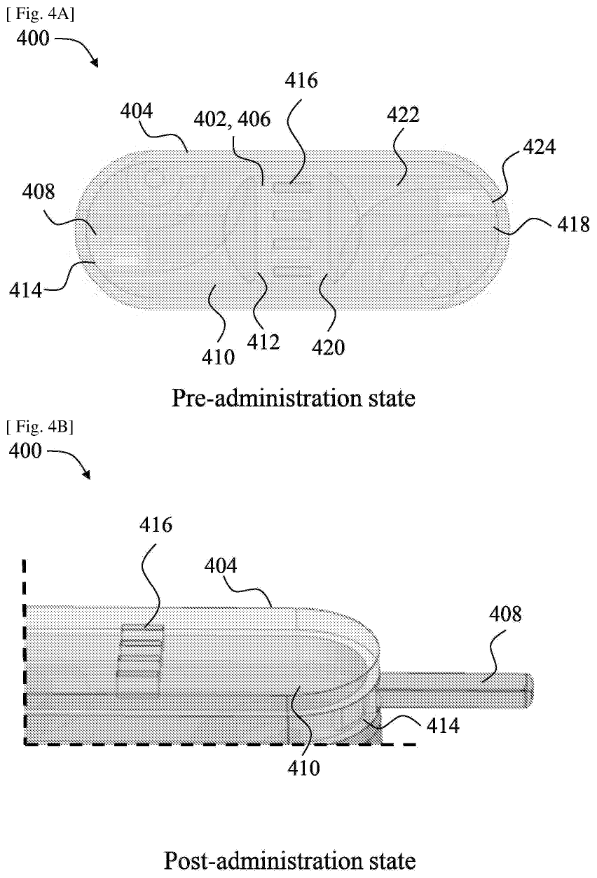

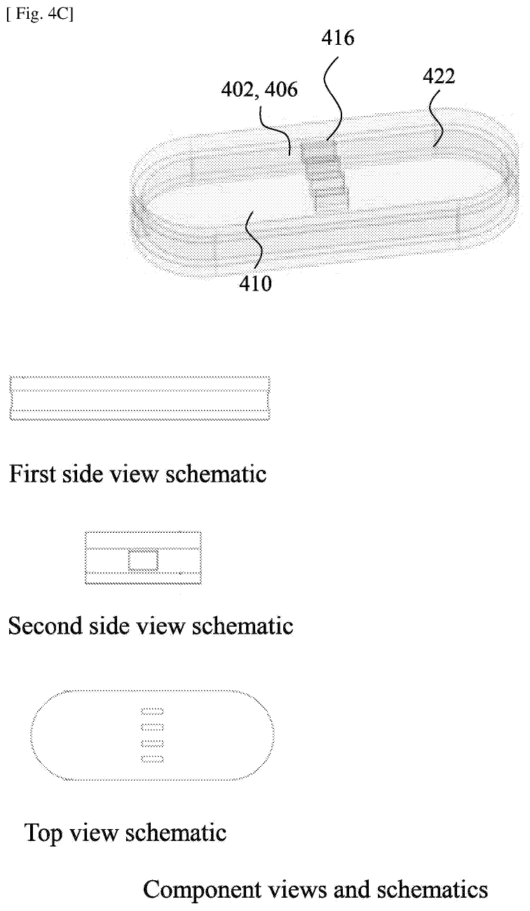

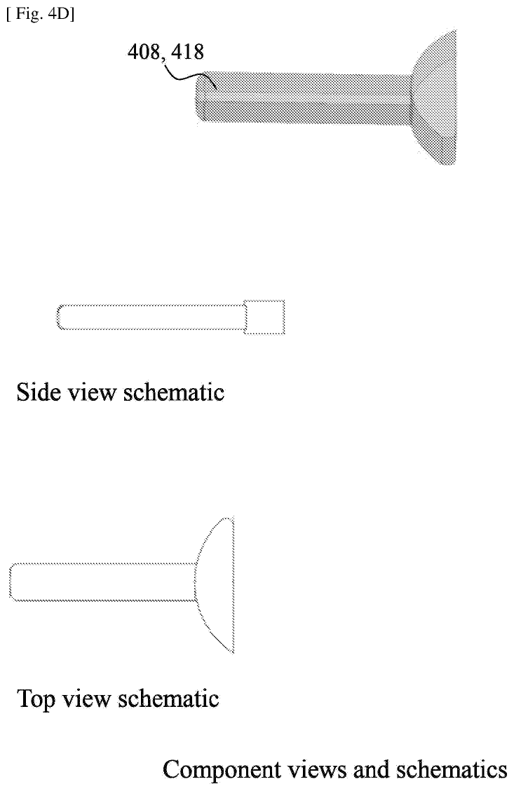

An example oral drug dosage form 400 configured for gastric retention is provided in FIGS. 4A-4C. As shown in a pre-administration state in FIG. 4A, the oral drug dosage form 400 comprises a swellable material 402 contained in a swellable material compartment 406 formed by a body 404 of the oral drug dosage form 400. The oral drug dosage form 400 comprises a first moveable arm 408 positioned within (or substantially within) the footprint of the oral drug dosage form 400 in the pre-administration state. The moveable arm 408 is configured such that at least a portion of the moveable arm is extendible beyond, or further away from, the body 404 of the oral drug dosage form 400 via a force that is provided by the swellable material 402. The body 404 of the oral drug dosage form 400 forms a directional channel 410 configured to guide the movement of the moveable arm 408 upon swelling of the swellable material 402. In the pre-administration state, the oral drug dosage form 400 is designed such that the moveable arm 408 comprises a contact element 412 configured to interface with the swellable material 402 in proximity to the directional channel 410. The oral drug dosage form 400 comprises a plurality (four are pictured) of fluid inlets in the form of pores 416 operably connected to the swellable material compartment 406. As the swellable material 402 swells, the swellable material 402 will push against the contact element 412 of the moveable arm 408 to move the moveable arm 408. The body 404 also forms a stop 414 configured to stop the moveable arm 408 at an extended position in the post-administration state. The oral drug dosage form 400 further comprises a second moveable arm 418 having a contact element 420 configured to interface with the swellable material 402. The second movable arm 418 is configured relative to a second directional channel 422, wherein the second movable arm 418 will move in the second directional channel 422 to a stop 424. A post-administration state of a portion of the oral drug dosage form 400 is shown in FIG. 4B. In the post-administration state, gastrointestinal fluids have entered the oral drug dosage form 400 via the fluid inlets comprising pores 416 such that the swellable material 402 swells and pushes the first movable arm 408 and the second moveable arm 418 to respective stops 414, 424 such that the first moveable arm 408 and second movable arm 418 are maintained at extended positions. Component views of the oral drug dosage form 400 are provided in FIGS. 4C and 4D. As shown in FIGS. 4C, certain oral drug dosage forms may have a seamless transition between a swellable material compartment and a directional channel, wherein a boundary can be assigned, for descriptive purposes, that is consistent with the teaching provided herein. In some embodiments, the components of the oral drug dosage form 400 may be produced, such as via three-dimensional printing, injection molding, ultrasonic welding, or any combination thereof, and then assembled to form the pre-administration oral drug dosage form 400. For example, in some embodiments, the body (or one or more portions thereof) is produced independently from the first moveable arm and second moveable arm, and such components are later assembled to form the oral drug dosage form.

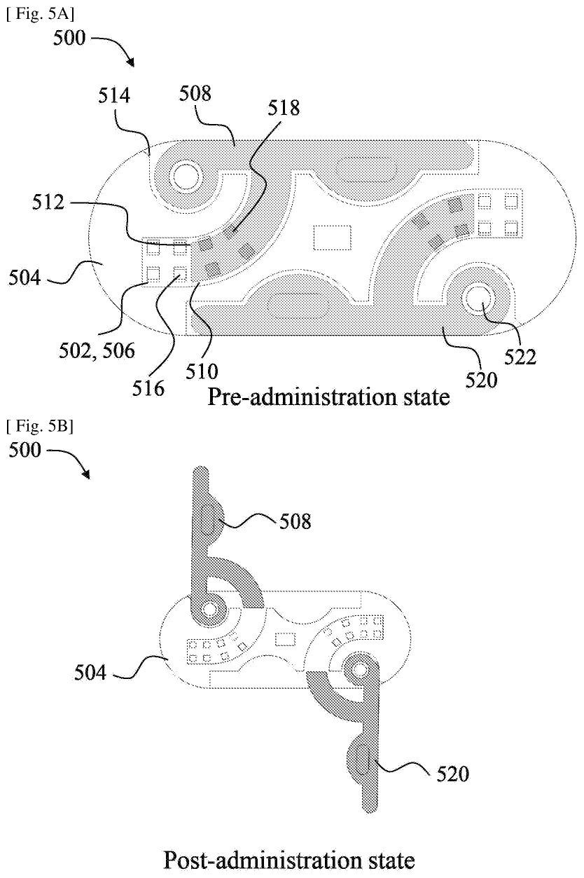

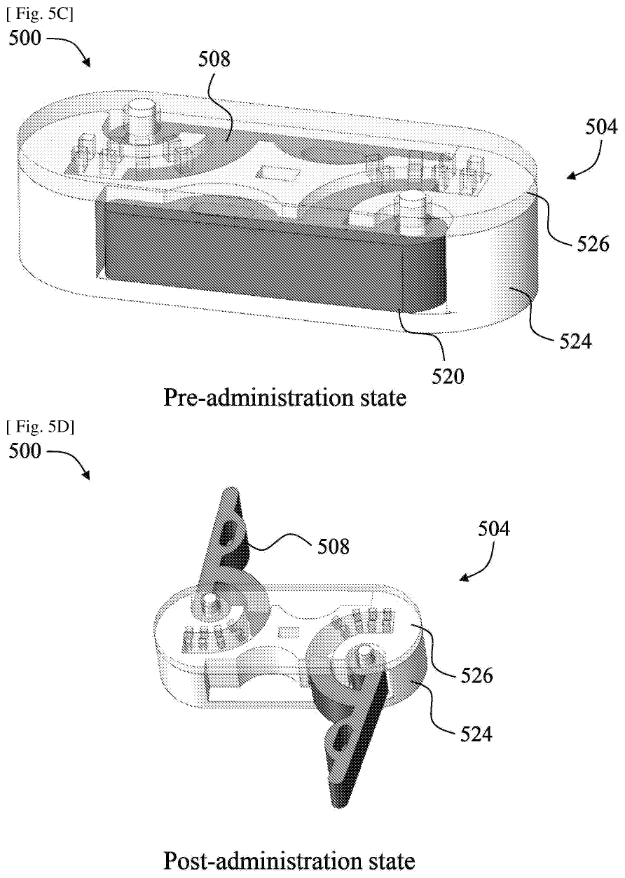

An example oral drug dosage form 500 configured for gastric retention is provided in FIGS. 5A-5F. As shown in a pre-administration state in FIG. 5A, the oral drug dosage form 500 comprises a first swellable material 502 contained in a first swellable material compartment 506 formed by a body 504 of the oral drug dosage form 500. The oral drug dosage form 500 comprises a first moveable arm 508 positioned within (or substantially within) the footprint of the oral drug dosage form 500 in the pre-administration state. The first moveable arm 508 is configured such that at least a portion of the first moveable arm is extendible beyond, or further away from, the body 504 of the oral drug dosage form 500 via a force that is provided by the first swellable material 502. The body 504 of the oral drug dosage form 500 forms a first directional channel 510 configured to guide the movement of the first moveable arm 508 upon swelling of the first swellable material 502. In the pre-administration state, the oral drug dosage form 500 is designed such that the first moveable arm 508 comprises a contact element 512 configured to interface with the first swellable material 502 in proximity to the first directional channel 510. The oral drug dosage form 500 comprises a plurality (four are pictured) of fluid inlets in the form of pores 516 operably connected to the first swellable material compartment 506. As the first swellable material 502 swells, the first swellable material 502 pushes against the contact element 512 of the first moveable arm 508 to move the first moveable arm 508. The first directional channel comprises a plurality (four are pictured) of fluid inlets in the form of pores 518 operably connected to the first directional channel 510 such that the first swellable material 502 continues to be exposed to gastrointestinal fluid as the first moveable arm 508 travels through the first directional channel 510. The body 504 also forms a first stop 514 configured to stop the first moveable arm 508 at an extended position in the post-administration state. The oral drug dosage form 500 further comprises a second moveable arm 520 having a matching movement mechanism as the first moveable arm 508. Both the first moveable arm and the second moveable arm are configured to rotate on an axis (such as 522 for the second moveable arm 520). A post-administration state of the oral drug dosage form 500 is shown in FIG. 5B. In the post-administration state, gastrointestinal fluids have entered the oral drug dosage form 500 via the fluid inlets comprising pores (such as 516 and 518 as relevant to the first swellable material) such that swellable material (such as the first swellable material 502 as relevant to the first moveable arm 508) pushes the movable arms 508, 520 to respective stops (such as the first stop 514 as relevant to the first moveable arm 508) such that the first moveable arm 508 and second movable arm 520 are configured at extended positions. Additional pre-administration state and post-administration state views of the oral drug dosage form 500 are provided in FIGS. 5C and 5D, respectively. As shown in FIGS. 5C and 5D, the body 504 of the oral drug dosage form 500 may comprise a frame 524 and a cap 526. Component views and accompanying schematics of certain aspects of the oral drug dosage form 500 are provided in FIGS. 5E and 5F. As shown in the frame view 524 of FIG. 5E, certain oral drug dosage forms may have a seamless transition between a swellable material compartment and a directional channel, wherein a boundary can be assigned, for descriptive purposes, that is consistent with the teaching provided herein. In some embodiments, the components of the oral drug dosage form 500 may be produced, such as via three-dimensional printing, injection molding, ultrasonic welding, or any combination thereof, and then assembled to form the pre-administration oral drug dosage form 500. For example, in some embodiments, the body (or one or more portions thereof) is produced independently from the first moveable arm and second moveable arm, and such components are later assembled to form the oral drug dosage form.

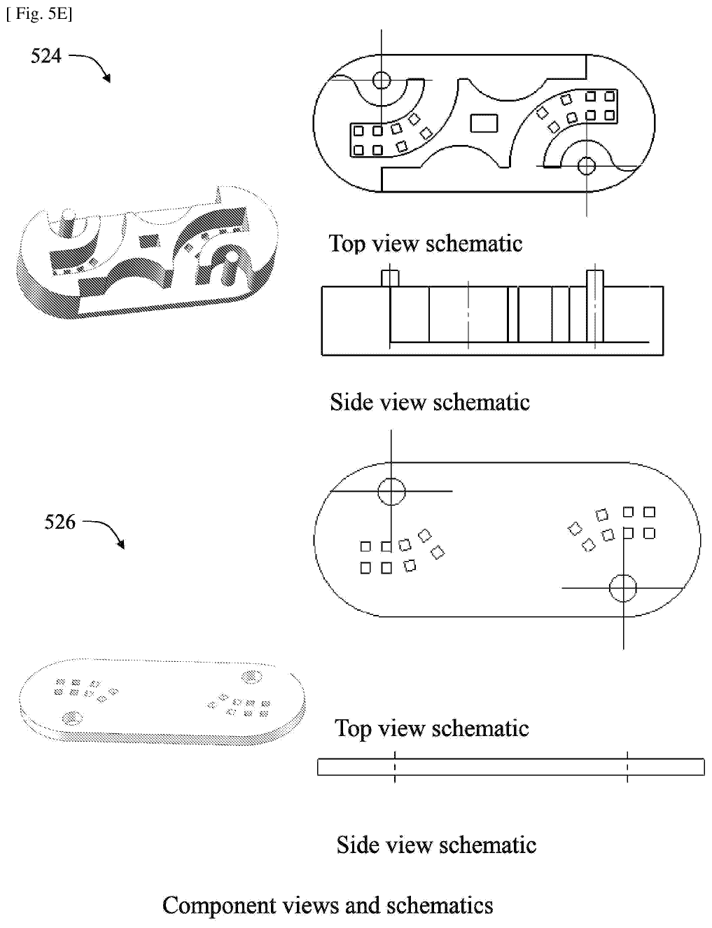

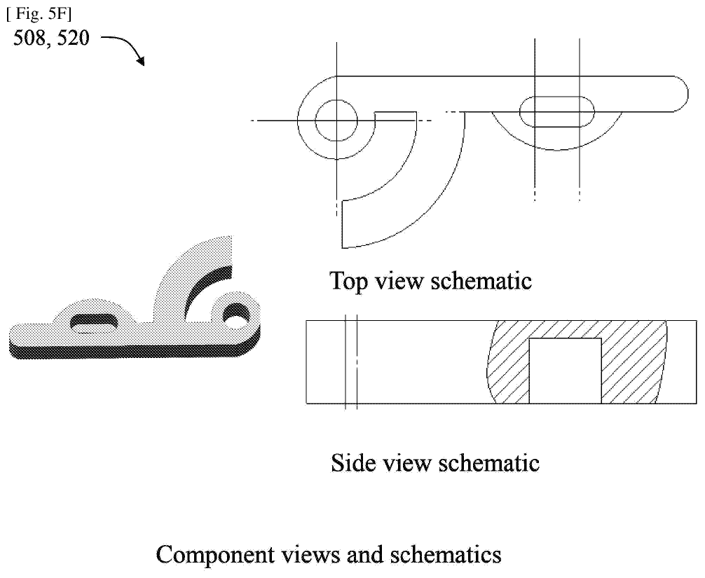

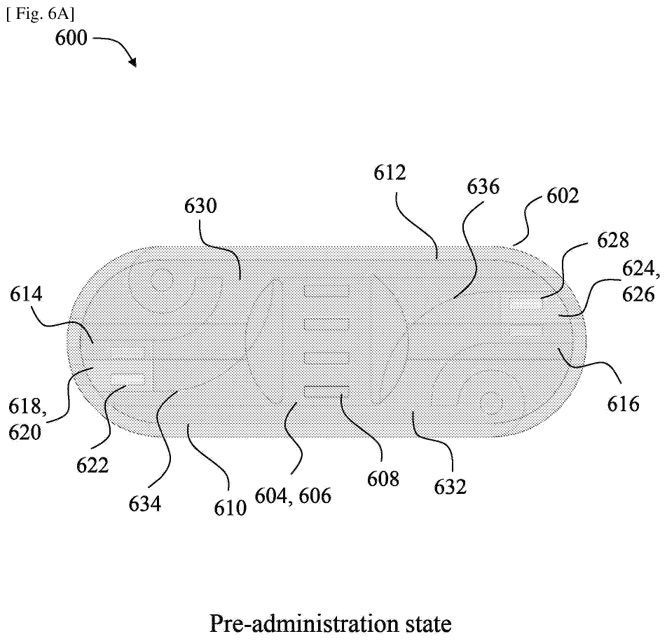

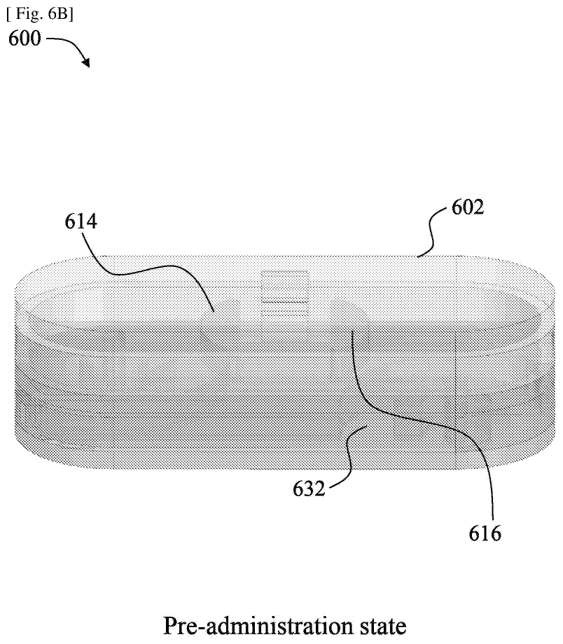

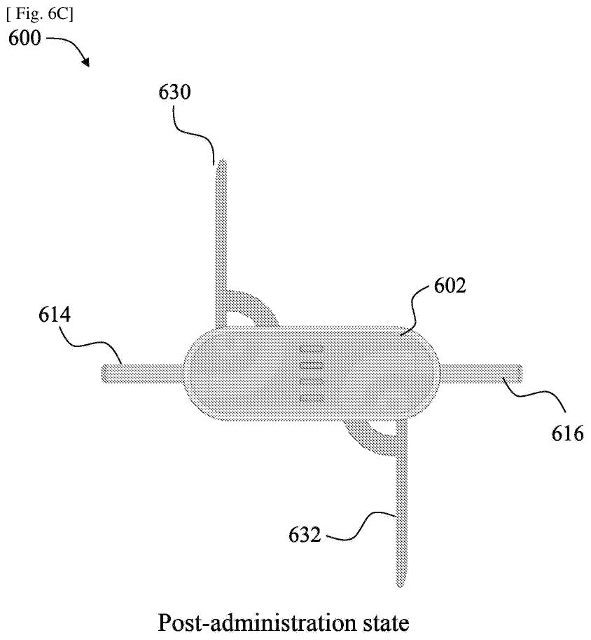

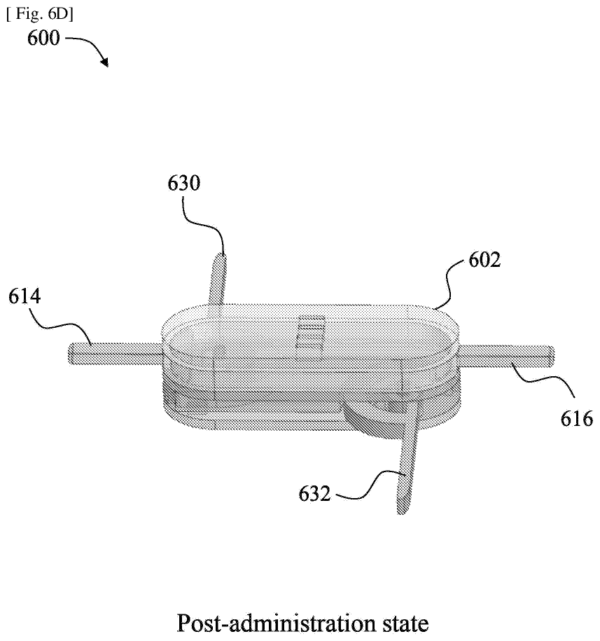

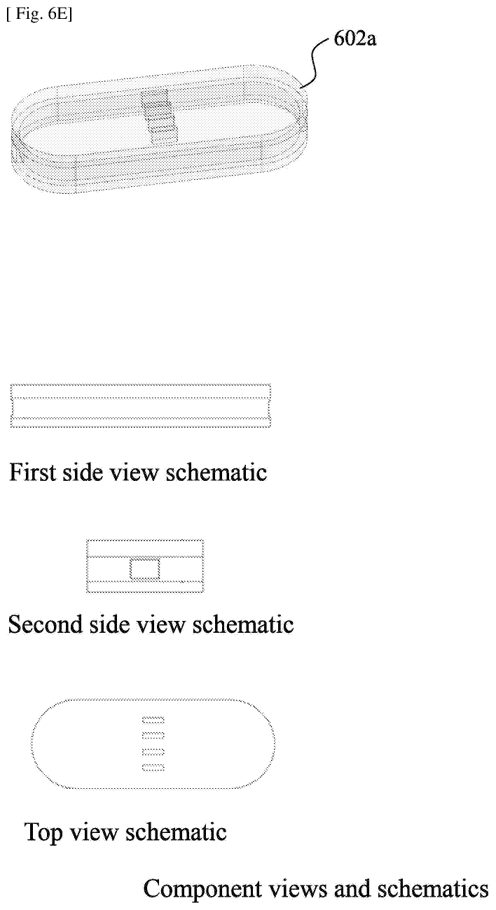

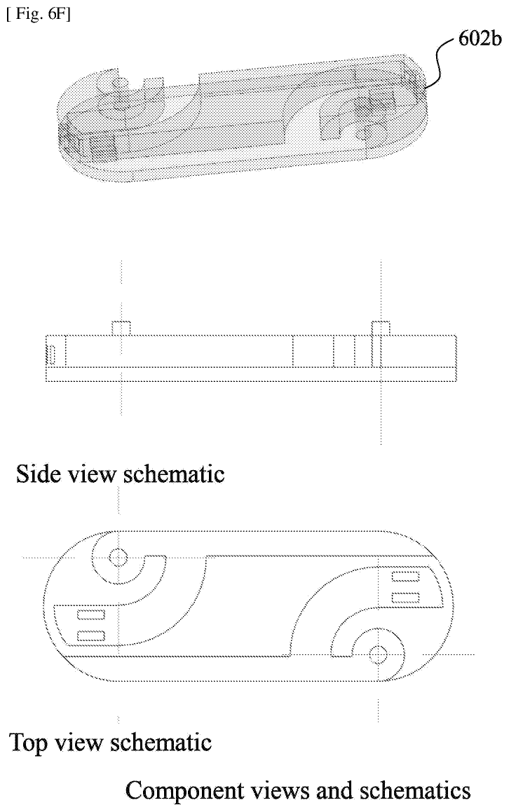

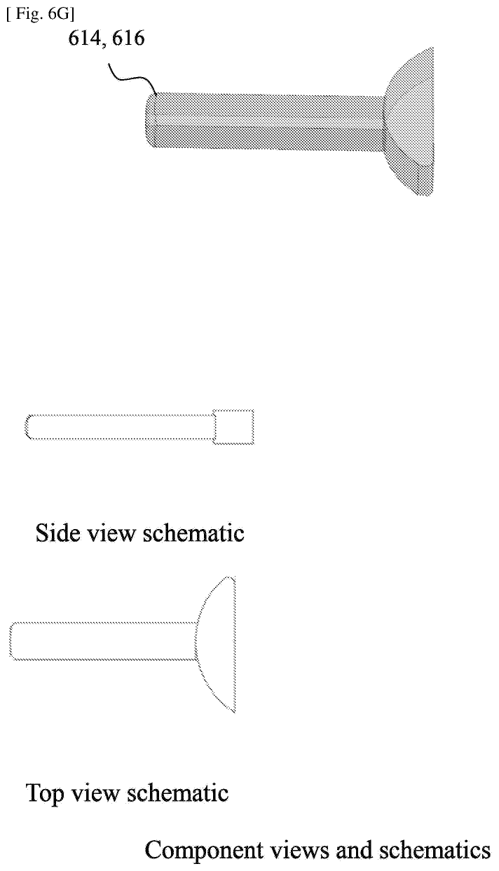

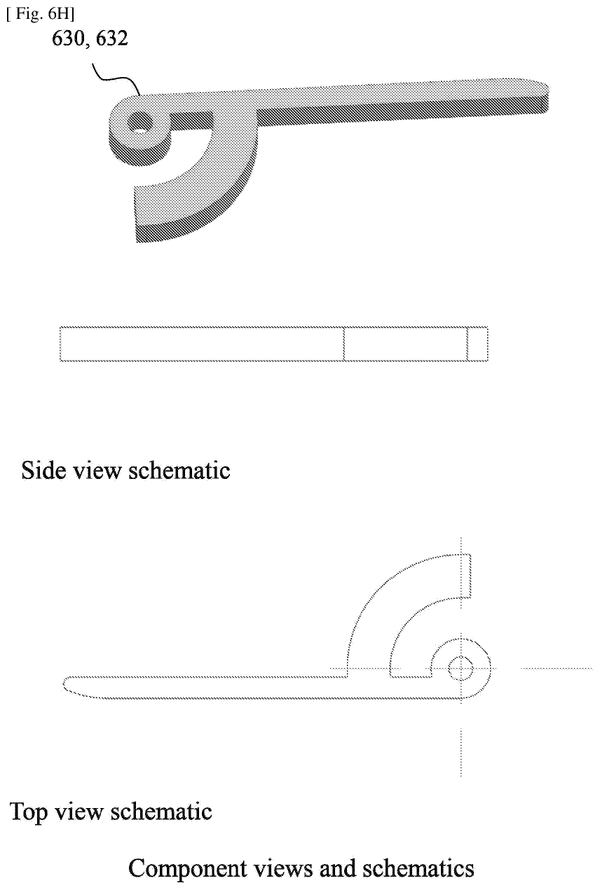

As taught herein, oral drug dosage forms may comprise moveable arms configured comprising different movement mechanisms. An example oral drug dosage form 600 configured for gastric retention is provided in FIGS. 6A-6H. As shown in a pre-administration state in FIG. 6A, the oral drug dosage form 600 comprises a body 602 comprising a first swellable material 604 in a first swellable material compartment 606. The body 602 comprises a first set of fluid inlets comprising pores 608 (four pictured) operably connected to the first swellable material compartment 606. The body 602 comprises a first directional channel 610 and a second directional channel 612 operably connected to the first swellable material compartment 606. A first moveable arm 614 is positioned in the first directional channel 610 and a second moveable arm 616 is positioned in the second directional channel 612. The oral drug dosage form 600 is configured in the pre-administration state such that the first moveable arm 614 and the second moveable arm 616 are positioned within (or substantially within) the footprint of the oral drug dosage form 600. The first moveable arm 614 and the second moveable arm 616 are configured such that they are extendible beyond, or further away from, the body 602 of the oral drug dosage form 600 via a force that is provided by the first swellable material 604. The first directional channel 610 and the second directional channel 612 are configured to guide the movement of the first moveable arm 614 and the second moveable arm 616 (respectively) upon swelling of the first swellable material 604. The body 602 of the oral drug dosage form 600 further comprises a second swellable material 618 in a second swellable material compartment 620. The body 602 comprises a second set of fluid inlets comprising pores 622 (two pictured) operably connected to the second swellable material compartment 620. The body 602 comprises a third directional channel 634 operably connected to the second swellable material compartment 620. A third moveable arm 614 is positioned in the third directional channel 634. The oral drug dosage form 600 is configured in the pre-administration state such that the third moveable arm 630 is positioned within (or substantially within) the footprint of the oral drug dosage form 600. The third moveable arm 630 is configured such that it is extendible beyond, or further away from, the body 602 of the oral drug dosage form 600 via a force that is provided by the second swellable material 618. The third directional channel 634 is configured to guide the movement of the third moveable arm 630 upon swelling of the second swellable material 618. The body 602 of the oral drug dosage form 600 further comprises a third swellable material 624 in a third swellable material compartment 626. The body 602 comprises a third set of fluid inlets comprising pores 628 (two pictured) operably connected to the third swellable material compartment 626. The body 602 comprises a fourth directional channel 636 operably connected to the third swellable material compartment 626. A fourth moveable arm 632 is positioned in the fourth directional channel 636. The oral drug dosage form 600 is configured in the pre-administration state such that the fourth moveable arm 632 is positioned within (or substantially within) the footprint of the oral drug dosage form 600. The fourth moveable arm 632 is configured such that it is extendible beyond, or further away from, the body 602 of the oral drug dosage form 600 via a force that is provided by the third swellable material 624. The fourth directional channel 636 is configured to guide the movement of the fourth moveable arm 632 upon swelling of the third swellable material 624. An alternative view of the pre-administration state of the oral drug dosage form 600 is provided in FIG. 6B, wherein only certain features are labeled to guide points for understanding the features of the oral drug dosage form 600. A post-administration state of the oral drug dosage form 600 is provided in FIG. 6C, illustrating the first moveable arm 614, the second moveable arm 616, the third moveable arm 630, and the fourth moveable arm 632 in extended positions beyond the body 602 of the oral drug dosage form 600. An alternative view of the post-administration state of the oral drug dosage form 600 is provided in FIG. 6D, wherein only certain features are labeled to guide points for understanding the features of the oral drug dosage form 600. Component views and schematics of the oral drug dosage form 600 are provided in FIGS. 6E-6H. As shown in FIGS. 6E and 6F, the body 602 may be composed of two or more modules, such as 602a and 602b. FIG. 6E shows the body 602a relevant to the first moveable arm and the second moveable arm. FIG. 6F shows the body 602b relevant to the third moveable arm and the fourth moveable arm. FIG. 6G shows views and schematics of the first moveable arm and the second moveable arm. FIG. 6H shows views and schematics of the third moveable arm and the fourth moveable arm. In some embodiments, the components of the oral drug dosage form 600 may be produced, such as via three-dimensional printing, injection molding, ultrasonic welding, or any combination thereof, and then assembled to form the pre-administration oral drug dosage form 600. For example, in some embodiments, the body (or one or more portions thereof) is produced independently from the first moveable arm and second moveable arm, and such components are later assembled to form the oral drug dosage form.

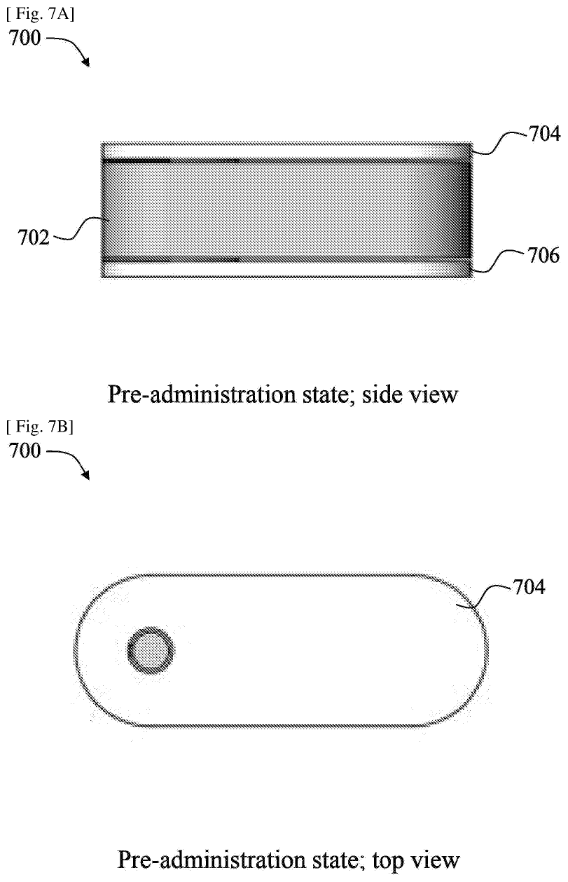

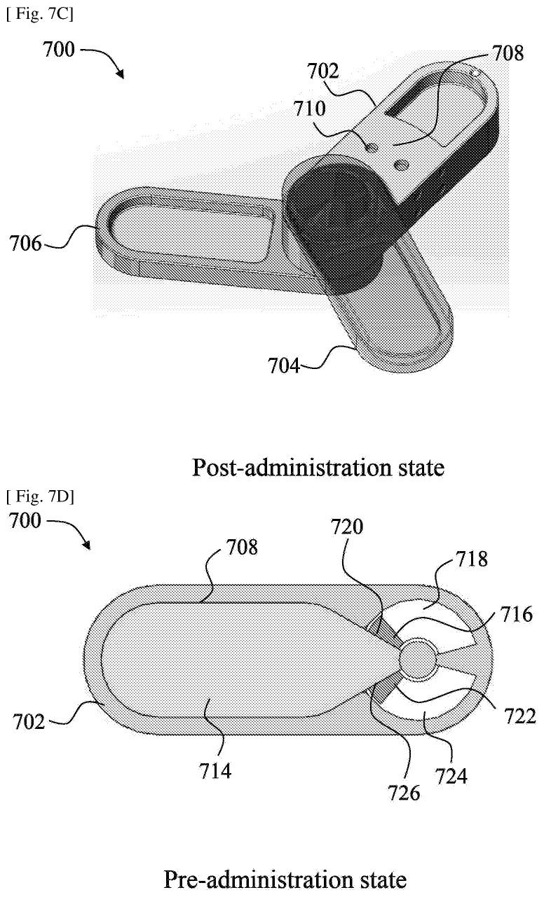

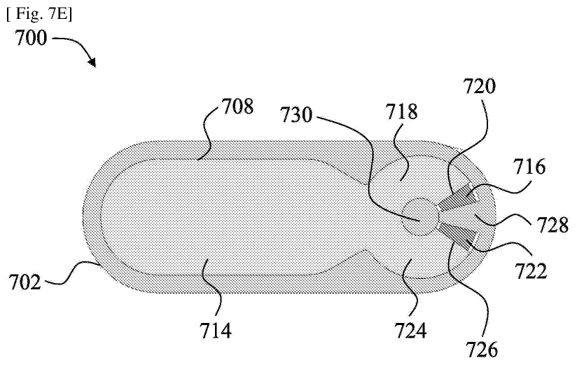

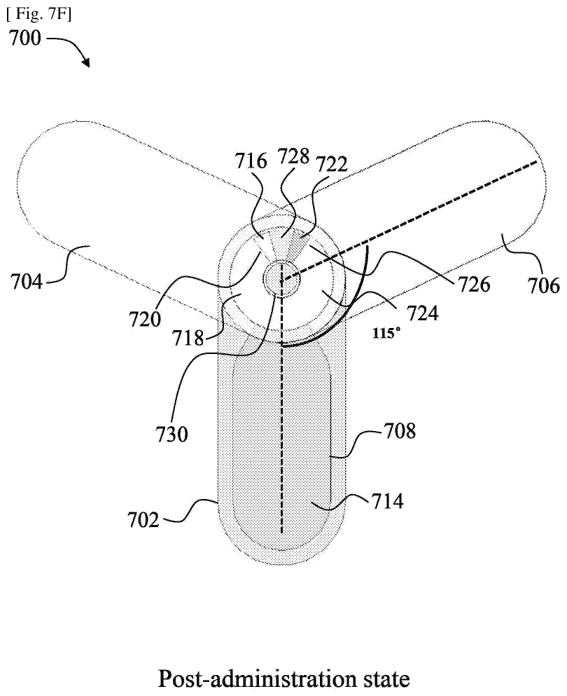

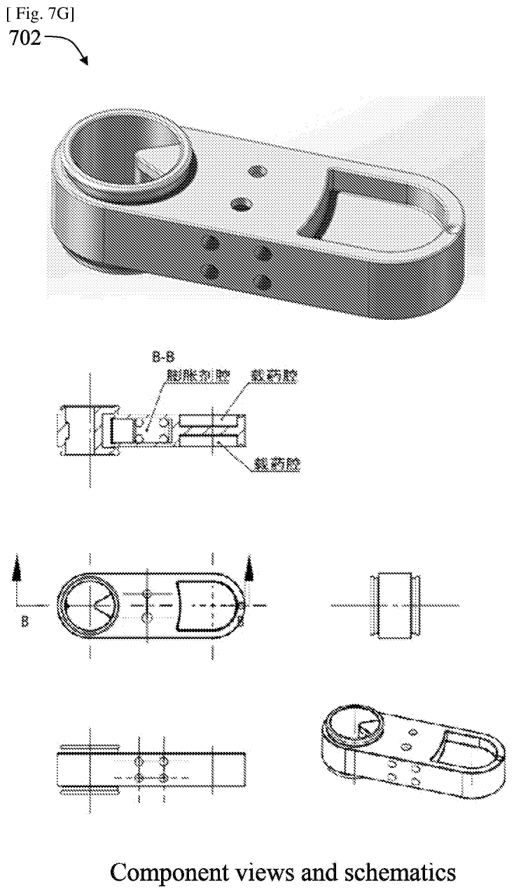

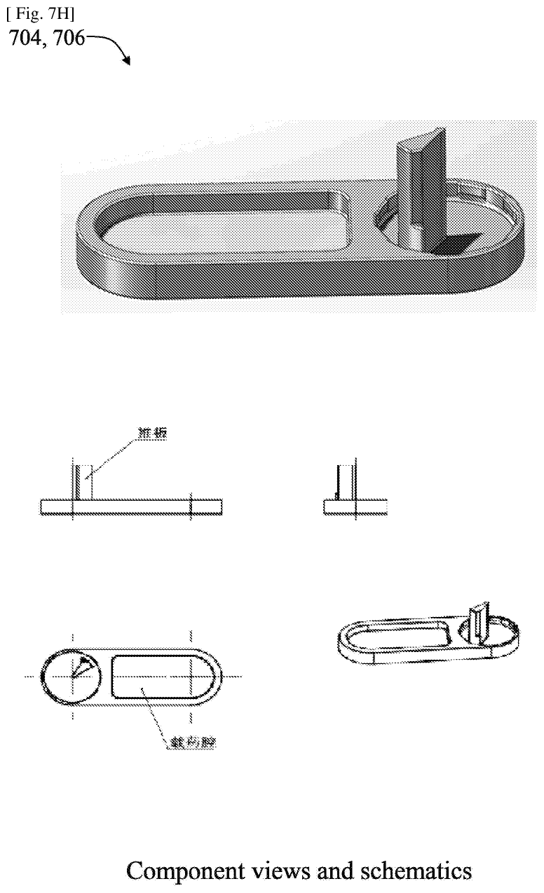

An example oral drug dosage form 700 is illustrated in FIGS. 7A-7I. As shown in FIGS. 7A and 7B, in a pre-administration state, the oral drug dosage form 700 comprises a body 702, a first moveable arm 704, and a second moveable arm 706 forming a capsule shaped oral drug dosage form. A post-administration state of the oral drug dosage form 700 is shown in FIG. 7C, wherein the first moveable arm 704 and the second moveable arm 706 are position beyond, or further away from the body 702 of the oral drug dosage form 700. In the post-administration state view, a set of fluid inlets in the form of pores 710 are shown on the body 702, the fluid inlets being operably connected to a swellable material compartment 708 comprising at least a part of a swellable material. To illustrate the mechanism of the oral drug dosage form 700, a cross-sectional view of a pre-administration state (FIG. 7D) and a cross-sectional view of a post-administration state (FIG. 7E) of the oral drug dosage form 700 are provided and further discussed. As shown in FIG. 7D, the body 702 of the oral drug dosage form 700 comprises a swellable material compartment 708, a first direction channel 718, and a second direction channel 724. The swellable material compartment 708 comprises a swellable material 714. In FIG. 7D, the first arm (not fully pictured) comprises a rudder-shaped feature 716 configured to travel in the first directional channel 718, the rudder-shaped feature 716 comprising a contact element 720 configured to interface with the swellable material 714. Further illustrated in FIG. 7D, the second arm (not fully pictured) comprises a rudder-shaped feature 722 configured to travel in the second directional channel 724, the rudder-shaped feature 722 comprising a contact element 726 configured to interface with the swellable material 714. As illustrated in FIG. 7E, in a post-administration state the swellable material 714 has expanded into the first directional channel 718 and the second directional channel 724 and exerted a force on the first moveable arm and the second moveable arm via each respective rudder-shaped feature. The oral drug dosage form 700 is configured such that the first moveable arm and the second moveable arm rotate on an axis 730 until reaching a stop 728. Specifically, the rudder-shaped feature 716 of the first moveable arm and the rudder-shaped feature 722 of the second moveable arm travel in respective directional channels leading to respective moveable arms extending beyond, or further away from, a body 702 of the oral drug dosage form 700. An additional view of the oral drug dosage form 700 is provided in FIG. 7F illustrating the first moveable arm 704 and the second moveable arm 706 in an extended post-administration state, wherein the rudder-shaped features 716, 722 of the first moveable arm 704 and the second moveable arm 706 have been pushed by the swellable material 714 to come into contact with the stop 728. The size of the oral drug dosage form 700 in the post-administration state provides gastric retention as the increased size does not readily pass through the pylorus. Component views and schematics of aspects of the oral drug dosage form 700 are provided in FIGS. 7G-7I. In some embodiments, the components of the oral drug dosage form 700 may be produced, such as via three-dimensional printing, injection molding, ultrasonic welding, or any combination thereof, and then assembled to form the pre-administration oral drug dosage form 700. For example, in some embodiments, the body (or one or more portions thereof) is produced independently from the first moveable arm and second moveable arm, and such components are later assembled to form the oral drug dosage form.

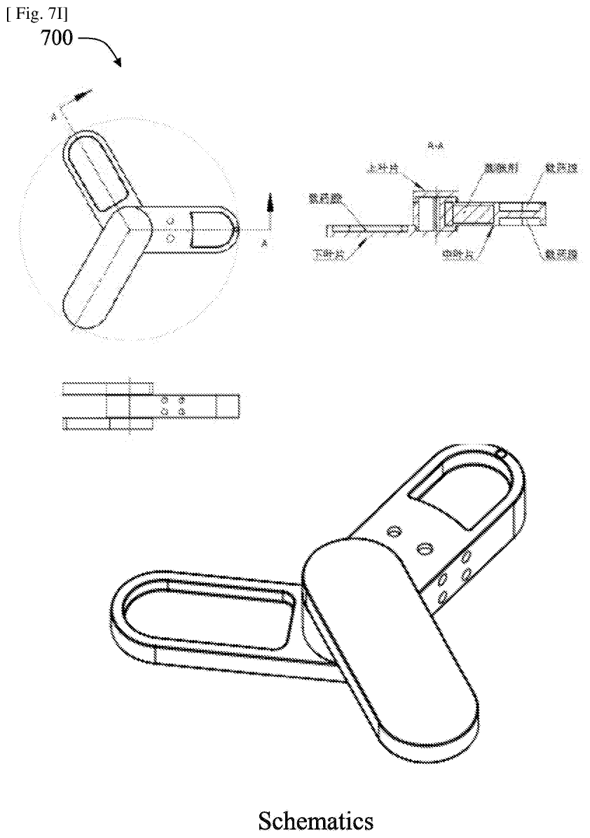

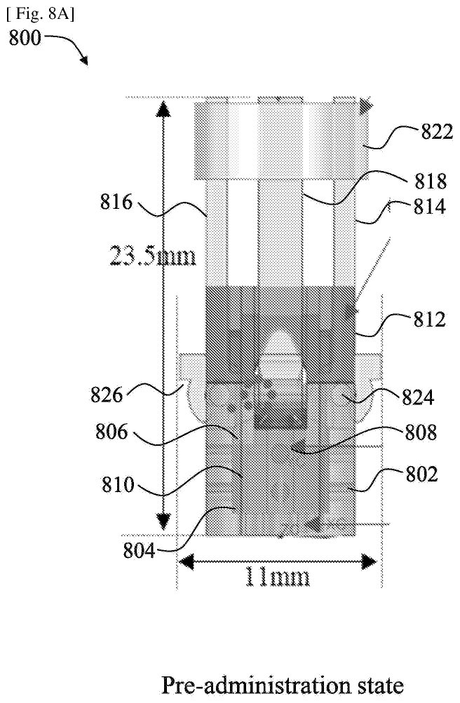

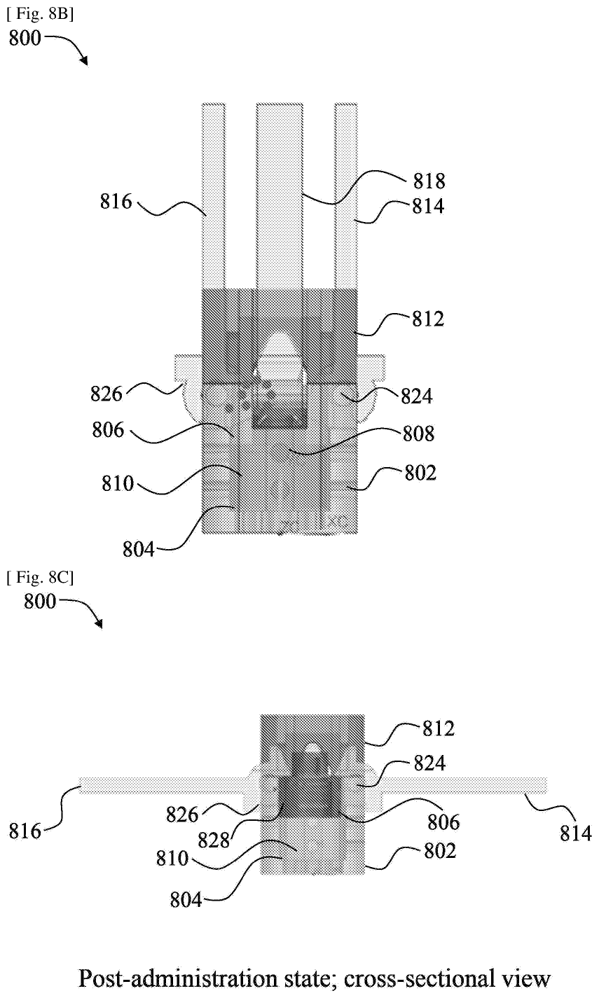

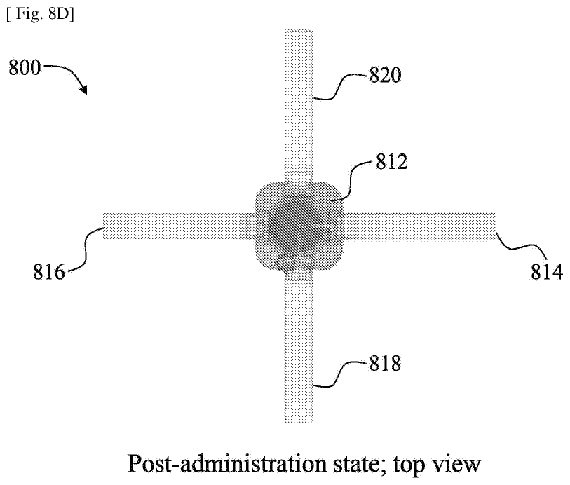

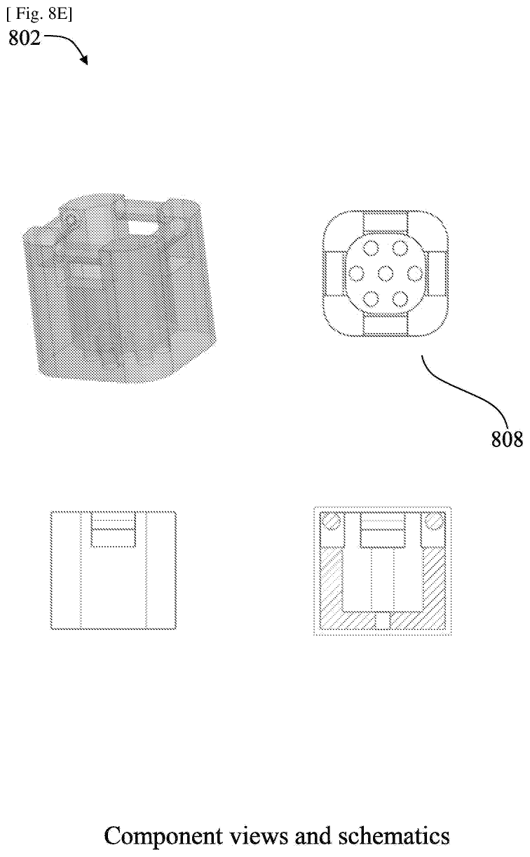

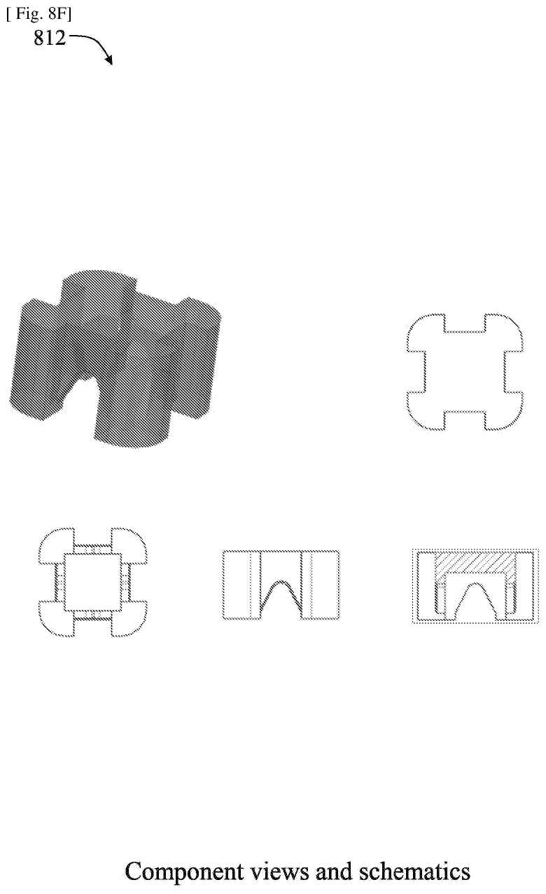

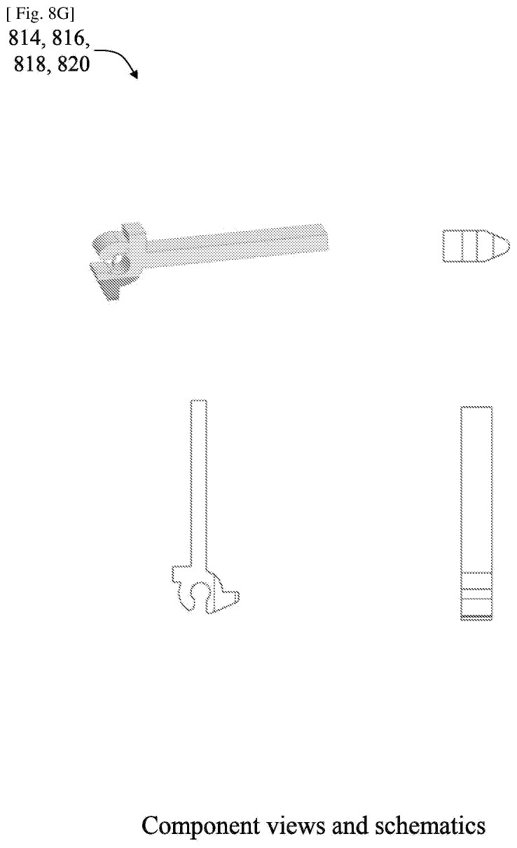

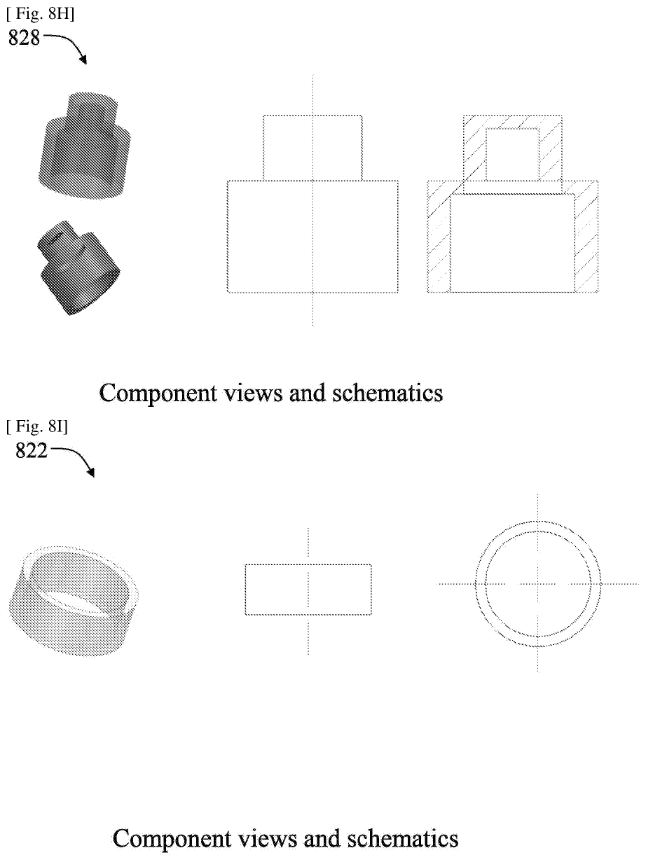

An example dosage form having four movable arms is provided in FIGS. 8A-8I. A pre-administration state of the oral drug dosage form 800 is provided in FIG. 8A, wherein the oral drug dosage form 800 comprises a body 802 having a swellable material compartment 804 configured to contain at least a portion of the swellable material 810. The body comprises a set of fluid inlets 808 operably connected to the swellable material compartment 804. The body 802 comprises a directional channel 806 operably connected to the swellable material compartment 804, wherein upon swelling the swellable material 810 may swell in the direction guided by the directional channel 806. The swellable material 810 may be configured in conjunction with a non-swellable feature, such as to maintain a certain shape of at least a portion of the swellable material and/or contact a contact element of one or more moveable arms. As illustrated in FIG. 8A, the swellable material is embedded in a non-swellable feature covering the upper portion of the swellable material in the pre-administration state (as later shown in FIG. 8C, the non-swellable feature 828 covering the upper portion of the swellable material 810 has been force upward and contacts each of the moveable arms). The body of the oral drug dosage form 800 comprises a cap 812. The oral drug dosage form 800 comprises four moveable arms (the view of FIG. 8A shows three of the moveable arms 814, 816, 818), each configured such that at least a portion of each moveable arm is extendible beyond, or further away from, the body of the oral drug dosage form 800 via a force that is provided by the swellable material 810. Specifically, each moveable arm is configured to rotate on an axis. For example, the first moveable arm 814 is configured to rotate on a first axis 824. The moveable arms may contain a feature 826 configured to engage with a stop of the oral drug dosage form 800. As shown in a pre-administration state shown in FIG. 8A, the oral drug dosage form may further comprise an erodible restrainer 822 configured to inhibit extension of moveable arms in a pre-administration state. The oral drug dosage form 800 illustrated in FIG. 8B does not have the erodible restrainer—this may be an embodiment of a pre-administration state of the oral drug dosage form 800 or a post-administration state of the oral drug dosage form 800 wherein the erodible restrainer has eroded but the moveable arms are yet to move to an extended position. A cross-sectional view of the oral drug dosage form 800 is provided in FIG. 8C, wherein the swellable material 810 has swelled as guided by the directional channel 806 of the body 802 exerting a force (via a non-swellable feature 828 covering the upper portion of the swellable material 810) on each moveable arm such that each moveable arm moves to an extended position until reaching the stop 826. FIG. 8D shows a top view of a post-administration state of the oral drug dosage form 800, wherein the first moveable arm 814, the second moveable arm 816, the third moveable arm 818, and the fourth moveable arm 820 are shown in an extended position. Component views and schematics of aspects of the oral drug dosage form 800 are provided in FIGS. 8E-8I. In some embodiments, the components of the oral drug dosage form 800 may be produced, such as via three-dimensional printing, injection molding, ultrasonic welding, or any combination thereof, and then assembled to form the pre-administration oral drug dosage form 800. For example, in some embodiments, the body (or one or more portions thereof, such as the cap 812) is produced independently from each moveable arm, and such components are later assembled to form the oral drug dosage form.

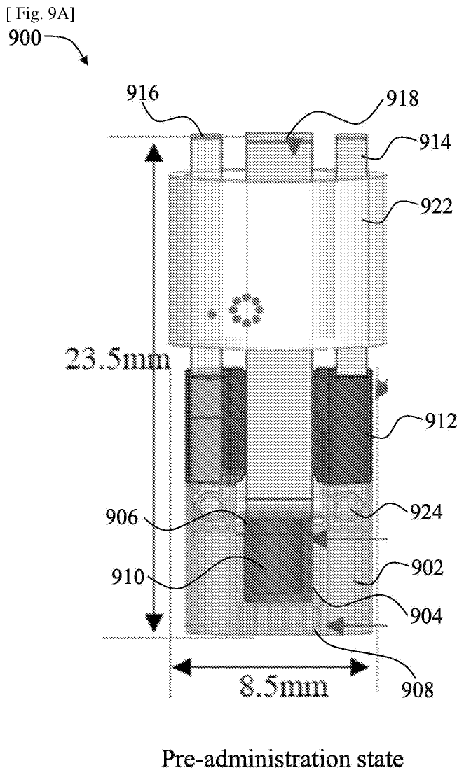

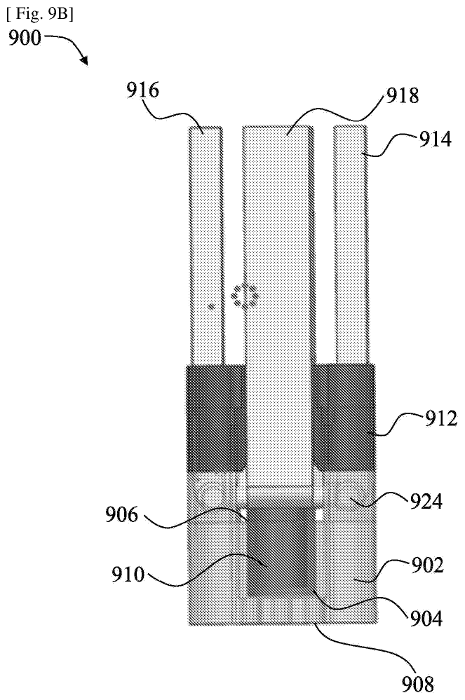

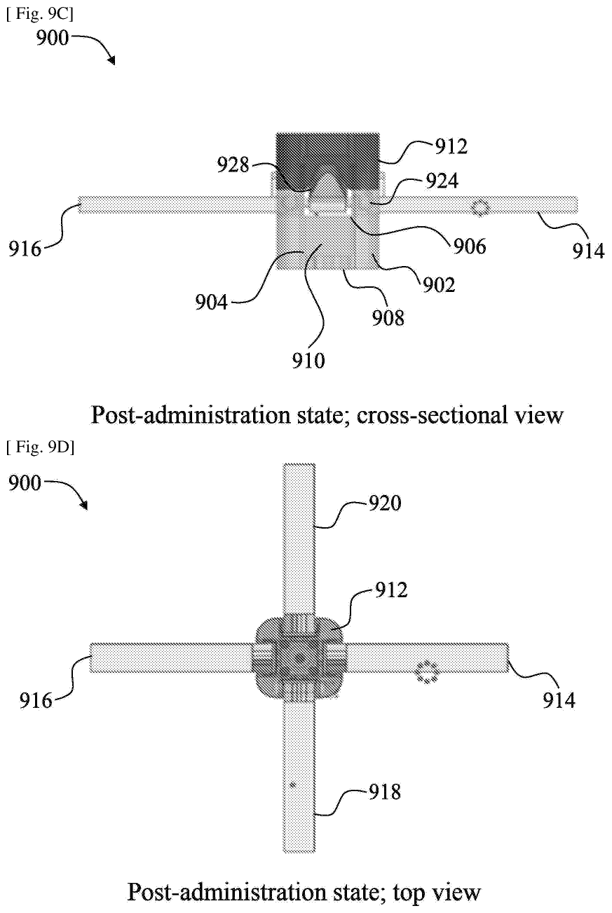

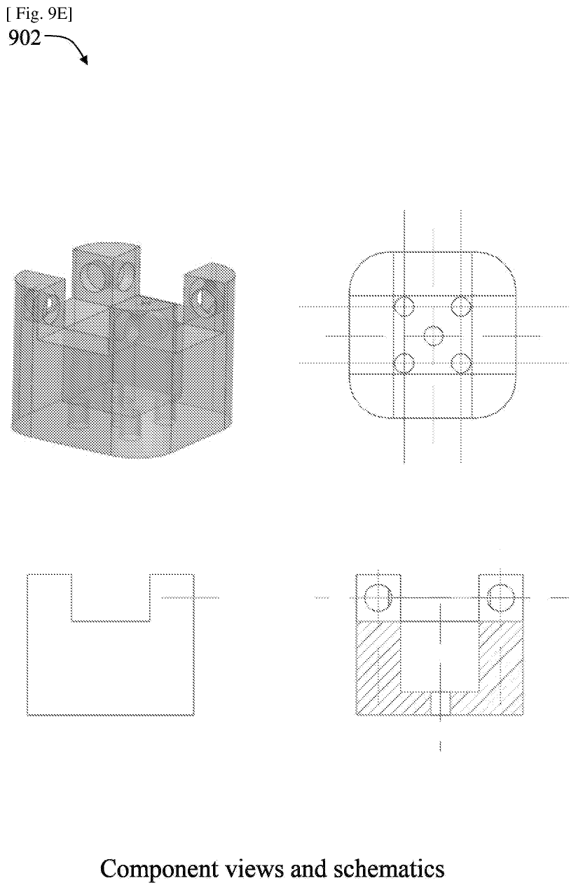

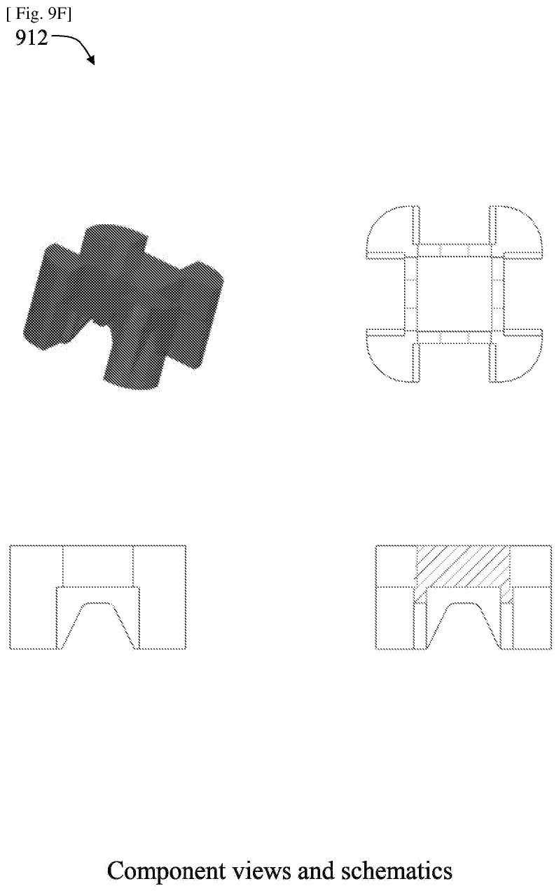

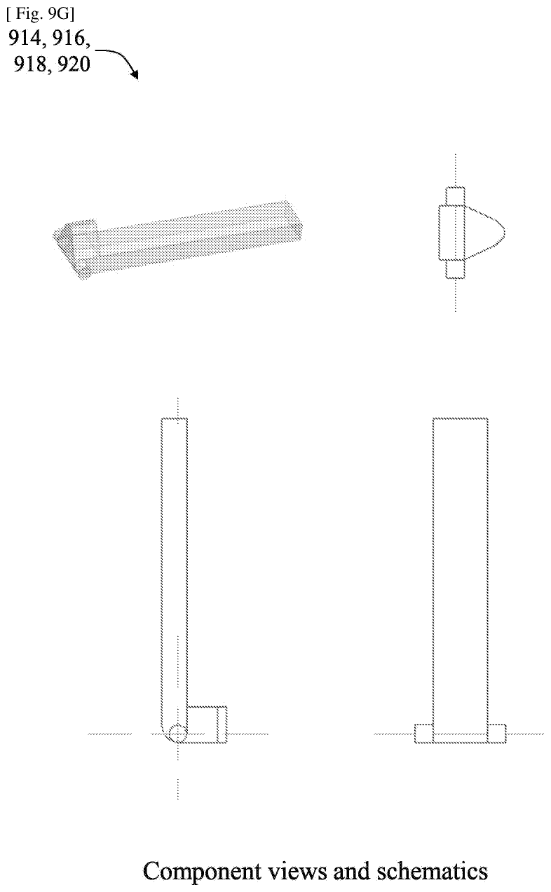

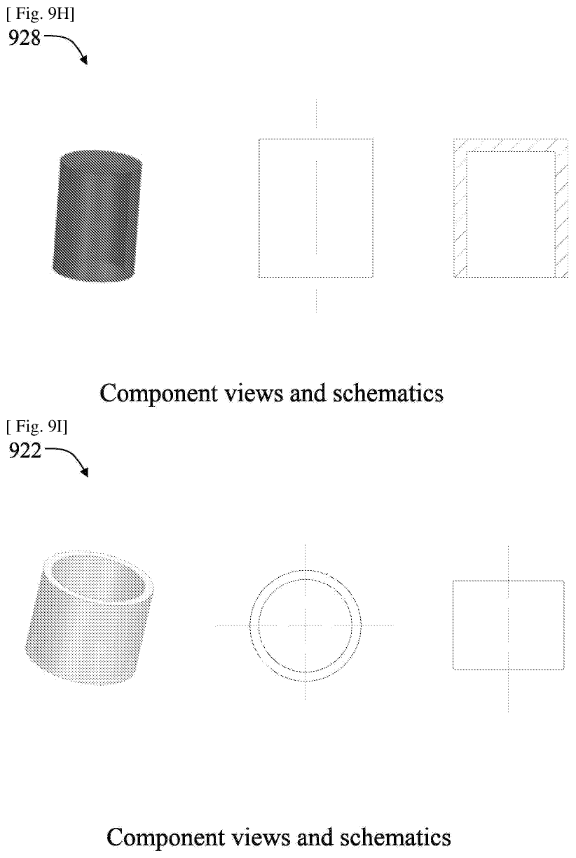

An example dosage form having four movable arms is provided in FIGS. 9A-9I. A pre-administration state of the oral drug dosage form 900 is provided in FIG. 9A, wherein the oral drug dosage form 900 comprises a body 902 having a swellable material compartment 904 configured to contain at least a portion of the swellable material 910. The body comprises a set of fluid inlets 908 operably connected to the swellable material compartment 904. The body 902 comprises an orifice 906 operably connected to the swellable material compartment 904, wherein upon swelling the swellable material 910 may extend out of and beyond the orifice 906 (or to at least to a greater degree if the swellable material already protrudes through the orifice). The swellable material 910 may be configured in conjunction with a non-swellable feature, such as to maintain a certain shape of at least a portion of the swellable material and/or contact a contact element of one or more moveable arms. As illustrated in FIG. 9A, the swellable material is embedded in a non-swellable feature covering the upper portion of the swellable material in the pre-administration state (as later shown in FIG. 9C, the non-swellable feature 928 covering the upper portion of the swellable material 910 has been force upward and contacts each of the moveable arms). The body of the oral drug dosage form 900 comprises a cap 912. The oral drug dosage form 900 comprises four moveable arms (the view of FIG. 9A shows three of the moveable arms 914, 916, 918), each configured such that at least a portion of each moveable arm is extendible beyond, or further away from, the body of the oral drug dosage form 900 via a force that is provided by the swellable material 910. Specifically, each moveable arm is configured to rotate on an axis. For example, the first moveable arm 914 is configured to rotate on a first axis 924. As shown in a pre-administration state shown in FIG. 9A, the oral drug dosage form 900 may further comprise an erodible restrainer 922 configured to inhibit extension of moveable arms in a pre-administration state. The oral drug dosage form 900 illustrated in FIG. 9B does not have the erodible restrainer—this may be an embodiment of a pre-administration state of the oral drug dosage form 900 or a post-administration state of the oral drug dosage form 900 wherein the erodible restrainer has eroded but the moveable arms are yet to move to an extended position. A cross-sectional view of the oral drug dosage form 900 is provided in FIG. 9C, wherein the swellable material 910 has swelled through and beyond (at least in part) of the orifice 906 of the body 902 exerting a force (via a non-swellable feature 928 covering the upper portion of the swellable material 910) on each moveable arm such that each moveable arm moves to an extended position. FIG. 9D shows a top view of a post-administration state of the oral drug dosage form 900, wherein the first moveable arm 914, the second moveable arm 916, the third moveable arm 918, and the fourth moveable arm 920 are shown in an extended position. Component views and schematics of aspects of the oral drug dosage form 900 are provided in FIGS. 9E-9I. In some embodiments, the components of the oral drug dosage form 900 may be produced, such as via three-dimensional printing, injection molding, ultrasonic welding, or any combination thereof, and then assembled to form the pre-administration oral drug dosage form 900. For example, in some embodiments, the body (or one or more portions thereof, such as the cap 912) is produced independently from each moveable arm, and such components are later assembled to form the oral drug dosage form.

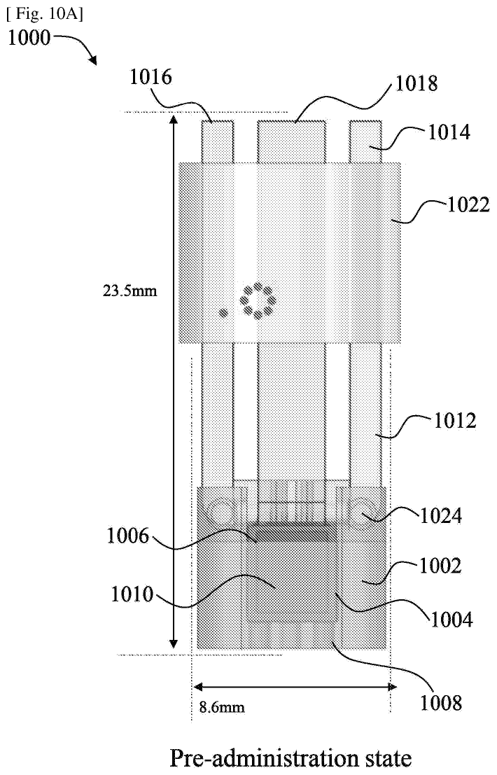

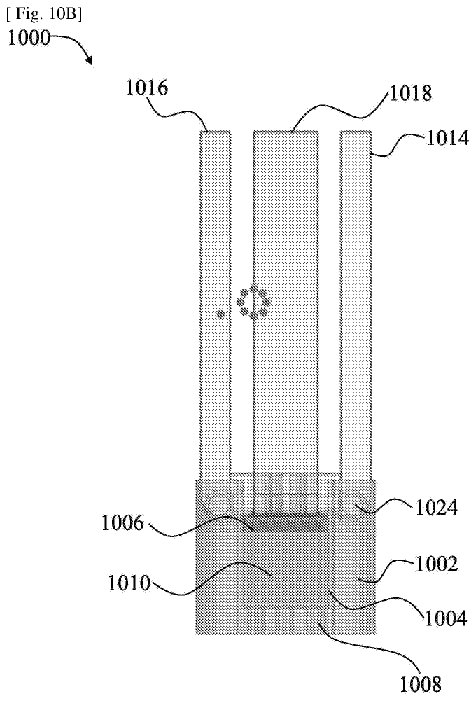

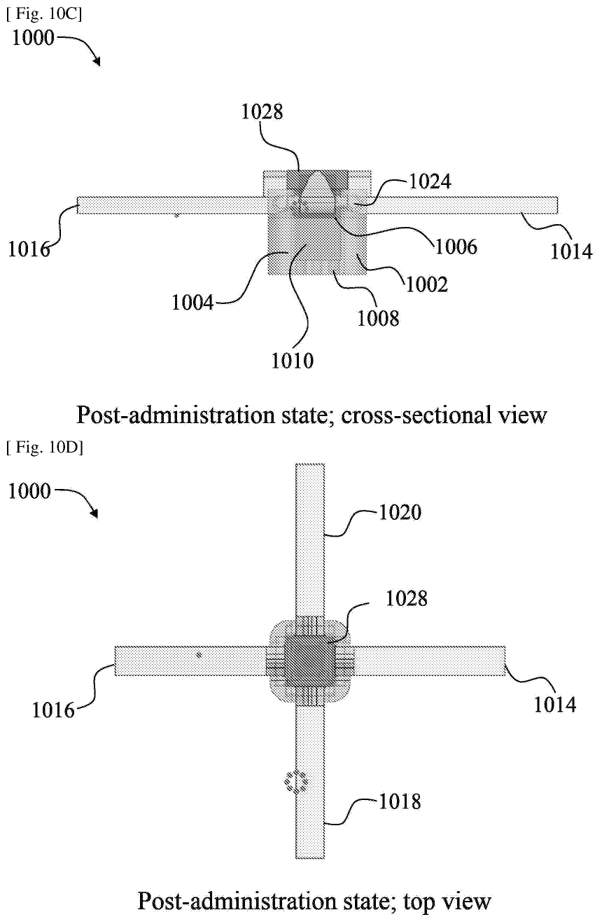

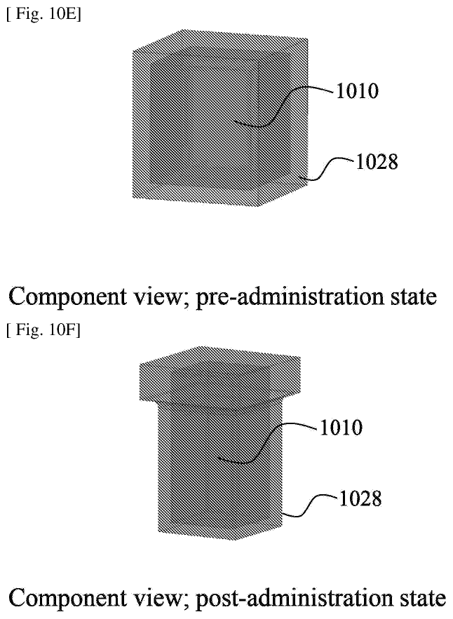

An example dosage form having four movable arms is provided in FIGS. 10A-10F. A pre-administration state of the oral drug dosage form 1000 is provided in FIG. 10A, wherein the oral drug dosage form 1000 comprises a body 1002 having a swellable material compartment 1004 configured to contain at least a portion of the swellable material 1010. The body comprises a set of fluid inlets 1008 operably connected to the swellable material compartment 1004. The body 1002 comprises an orifice 1006 operably connected to the swellable material compartment 1004, wherein upon swelling the swellable material 1010 may extend out of and beyond the orifice 1006 (or to at least to a greater degree if the swellable material already protrudes through the orifice). The swellable material 1010 may be configured in conjunction with a non-swellable feature, such as to maintain a certain shape of at least a portion of the swellable material and/or contact a contact element of one or more moveable arms. As illustrated in FIG. 10A, the swellable material is embedded in a non-swellable feature encapsulating the swellable material in the pre-administration state (as later shown in FIG. 10C, the non-swellable feature 1028 expands with the swellable material 1010 and contacts each of the moveable arms). The non-swellable feature may be or comprise a semi-permeable material that allows gastrointestinal fluid to enter the swellable material. In some embodiments, the oral drug dosage form may comprise one or more fluid inlets, wherein at least one of the fluid inlets is a pore. The oral drug dosage form 1000 comprises four moveable arms (the view of FIG. 10A shows three of the moveable arms 1014, 1016, 1018), each configured such that at least a portion of each moveable arm is extendible beyond, or further away from, the body of the oral drug dosage form 1000 via a force that is provided by the swellable material 1010. Specifically, each moveable arm is configured to rotate on an axis. For example, the first moveable arm 1014 is configured to rotate on a first axis 1024. As shown in a pre-administration state shown in FIG. 10A, the oral drug dosage form 1000 may further comprise an erodible restrainer 1022 configured to inhibit extension of moveable arms in a pre-administration state. The oral drug dosage form 1000 illustrated in FIG. 10B does not have the erodible restrainer—this may be an embodiment of a pre-administration state of the oral drug dosage form 1000 or a post-administration state of the oral drug dosage form 1000 wherein the erodible restrainer has eroded but the moveable arms are yet to move to an extended position. A cross-sectional view of the oral drug dosage form 1000 is provided in FIG. 10C, wherein the swellable material 1010 has swelled through and beyond (at least in part) of the orifice 1006 of the body 1002 exerting a force (via a non-swellable feature, i.e., a semi-permeable material 1028 surrounding the swellable material 1010) on each moveable arm such that each moveable arm moves to an extended position. FIG. 10D shows a top view of a post-administration state of the oral drug dosage form 1000, wherein the first moveable arm 1014, the second moveable arm 1016, the third moveable arm 1018, and the fourth moveable arm 1020 are shown in an extended position. Component views of the swellable material 1010 surrounded by the non-swellable feature 1028 are provided for a pre-administration state (FIG. 10E) and for a post-administration state (FIG. 10F) are provided. In some embodiments, the post-administration state forms a designed shape, provided by the swellable material and/or the non-swellable feature. In some embodiments, the components of the oral drug dosage form 1000 may be produced, such as via three-dimensional printing, injection molding, ultrasonic welding, or any combination thereof, and then assembled to form the pre-administration oral drug dosage form 1000. For example, in some embodiments, the body (or one or more portions thereof) is produced independently from each moveable arm, and such components are later assembled to form the oral drug dosage form.

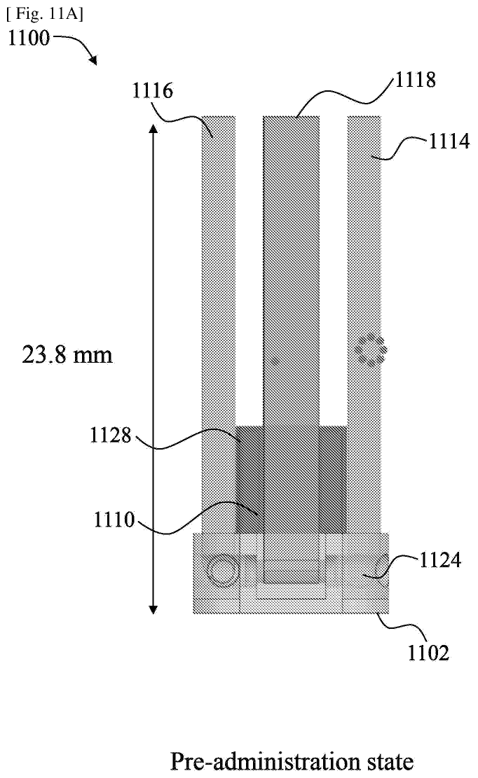

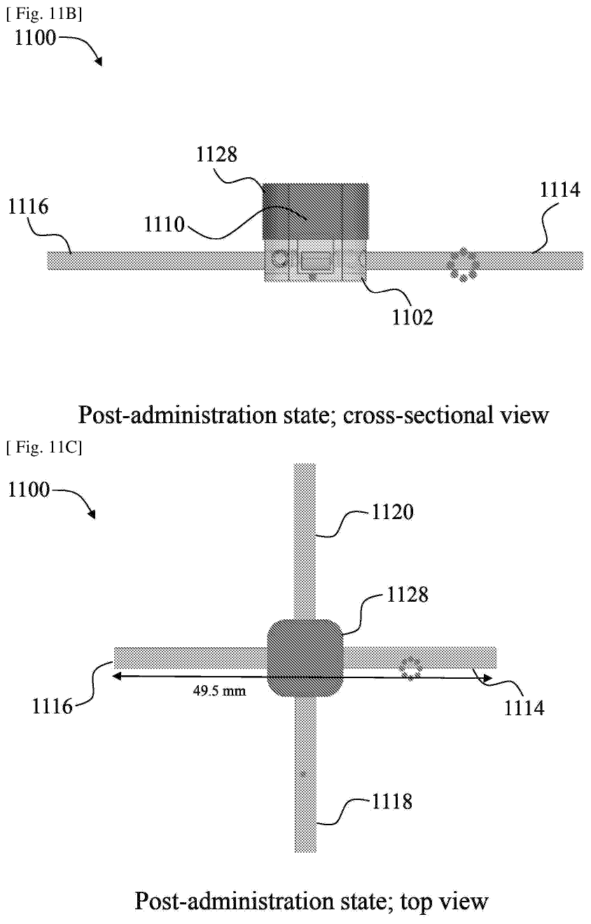

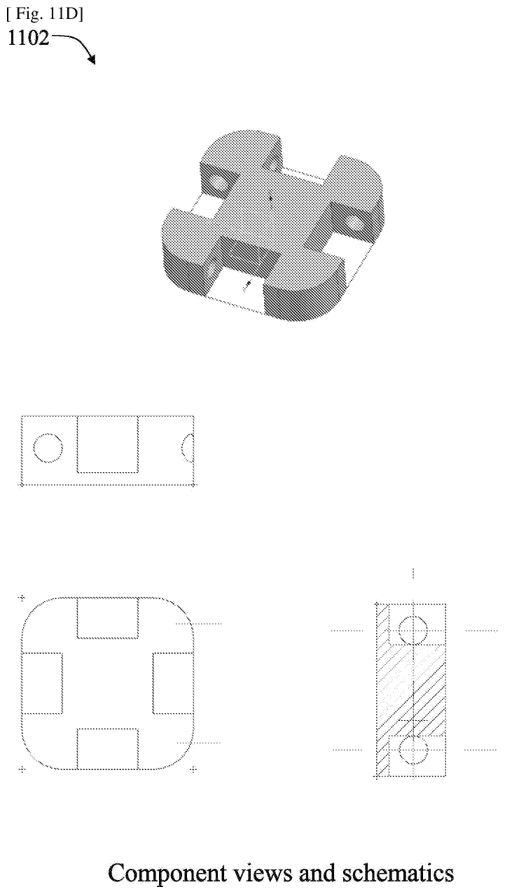

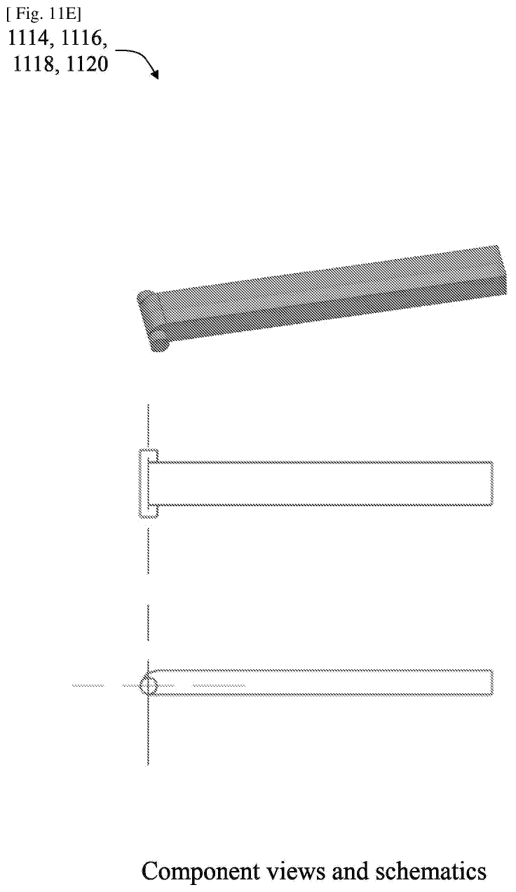

An example dosage form having four movable arms is provided in FIGS. 11A-11E. A pre-administration state of the oral drug dosage form 1100 is provided in FIG. 11A, wherein the oral drug dosage form 1100 comprises a body 1102 comprising points of connection for each movable arm. The oral drug dosage form 1100 comprises four moveable arms (the view of FIG. 11A shows three of the moveable arms 1114, 1116, 1118), each configured such that at least a portion of each moveable arm is extendible beyond, or further away from, the body of the oral drug dosage form 1100 via a force that is provided by the swellable material 1110. Each moveable arm is configured to rotate on an axis. For example, the first moveable arm 1114 is configured to rotate on a first axis 1124. The swellable material 1110 may be configured in conjunction with a non-swellable feature 1128, such as to maintain a certain shape of at least a portion of the swellable material and/or contact a contact element of one or more moveable arms. As illustrated in FIG. 11A, the swellable material 1110 and associated non-swellable feature 1128 are position on top of the body 1102 and between the moveable arms. In some embodiments, the oral drug dosage form may comprise an erodible restrainer around the moveable arms. The non-swellable material 1128 associated with the swellable material 1110 is a semi-permeable material that allows gastric fluid to enter the swellable material. In some embodiments, the oral drug dosage form may comprise one or more fluid inlets, wherein at least one of the fluid inlets is a pore. A cross-sectional view of the oral drug dosage form 1100 is provided in FIG. 11B, wherein the swellable material 1110 has swelled exerting a force (via a non-swellable feature 1128) on each moveable arm such that each moveable arm moves to an extended position. FIG. 11C shows a top view of a post-administration state of the oral drug dosage form 1100, wherein the first moveable arm 1114, the second moveable arm 1116, the third moveable arm 1118, and the fourth moveable arm 1120 are shown in an extended position. Component views and schematics of aspects of the oral drug dosage form 1100 are provided in FIGS. 11D and 11E. In some embodiments, the components of the oral drug dosage form 1100 may be produced, such as via three-dimensional printing, injection molding, ultrasonic welding, or any combination thereof, and then assembled to form the pre-administration oral drug dosage form 1100.

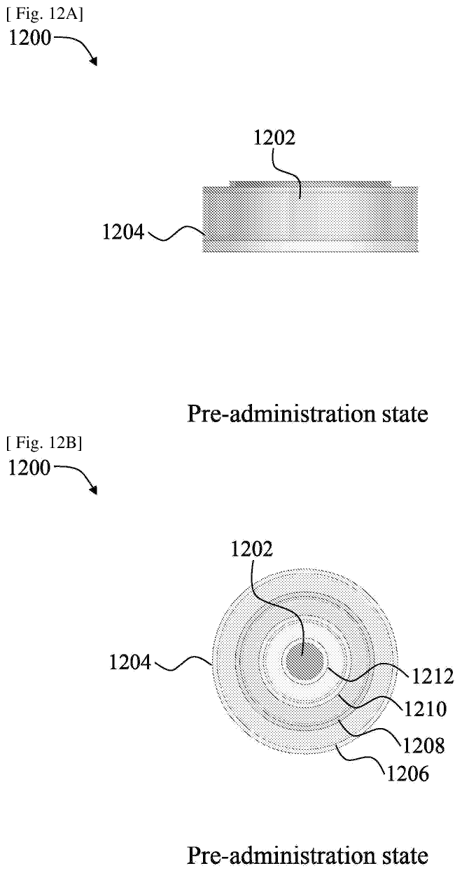

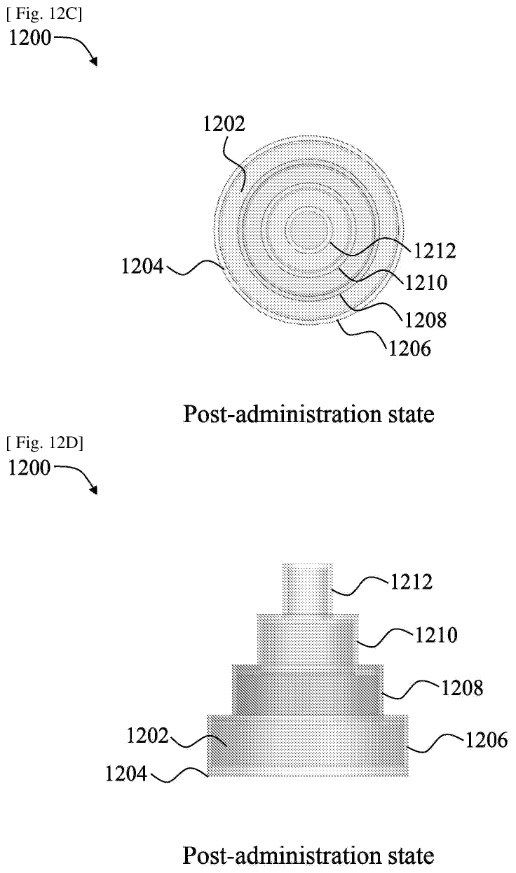

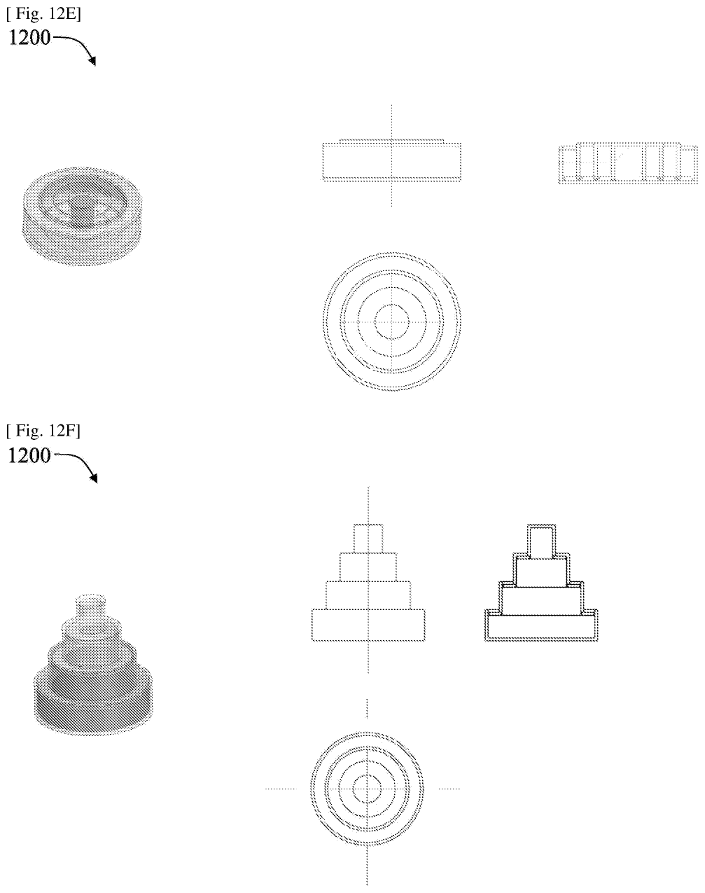

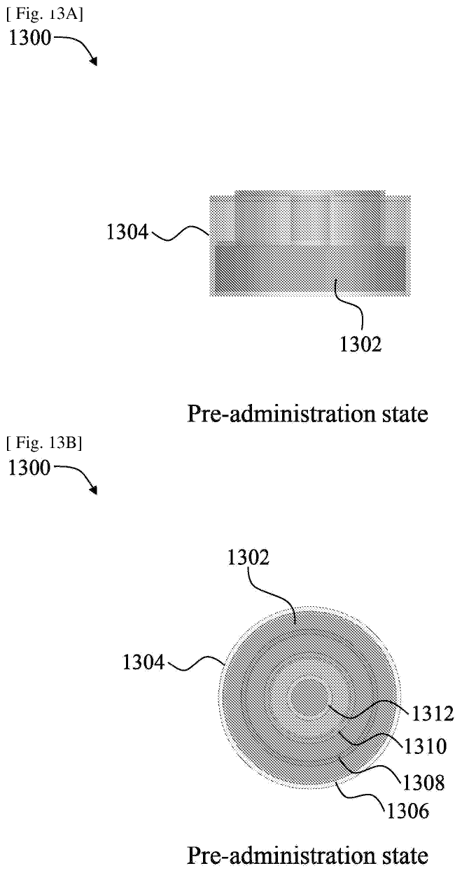

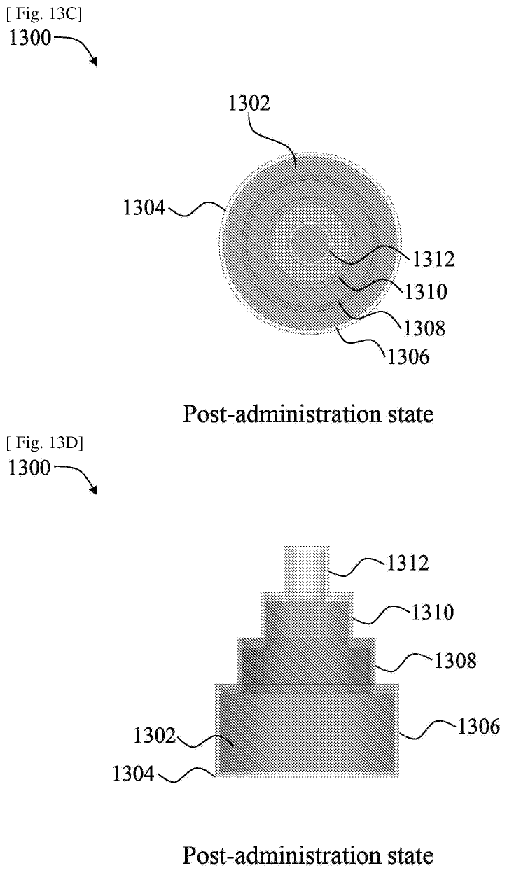

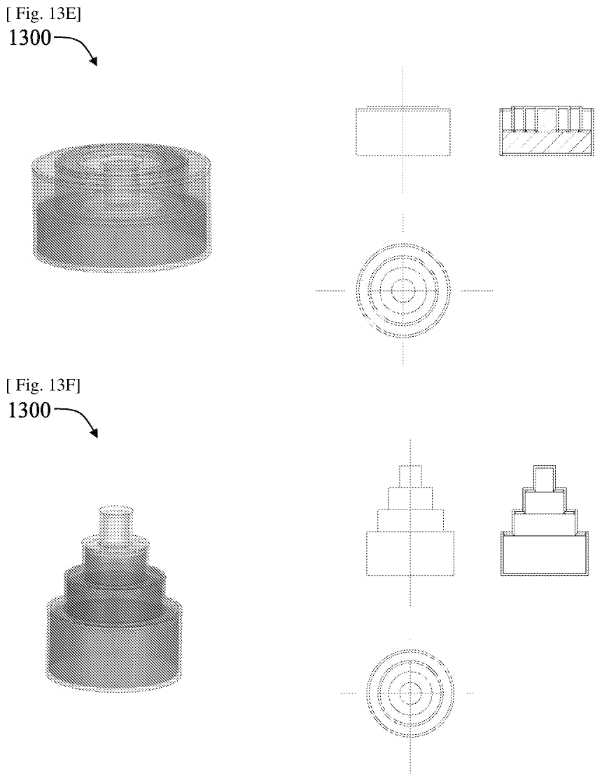

As taught herein, in some embodiments, the swellable material (and, optionally, any non-swellable feature associated therewith) may have a desired pre-administration state and a desired post-administration state. For example, in certain of the dosage forms taught herein, provided are swellable materials (and, optionally, any non-swellable feature associated therewith) that obtain a specific structure in a post-administration state after swelling occurs via exposure to gastrointestinal fluids. An example of this concept is provided in the swelling structure 1200 of FIGS. 12A-12E. In FIG. 12A, the swellable material 1202 and an associated shell 1204 are shown in a pre-administration state from a side view, wherein the swellable material 1202 is a core within the associated shell 1204 that forms the swelling structure 1200. The shell 1204 is configured to expand to a shape having at least two tiers having sequentially smaller outer dimensions. The associated shell 1204 comprises a means for water to access the swellable material 1202, e.g., a pore and/or a semi-permeable material. A top view of the swellable material 1202 and the associate shell 1204 are shown in FIG. 12B. As seen from the top view of FIG. 12B, there are four tiers 1206, 1208, 1210, and 1212 that nest together in the pre-administration state configured in a concentric arrangement. Post-administration state views of the swelling structure 1200 are provided in FIGS. 12C and 12D, wherein the swelling material 1202 has expanded to fill the associated shell 1204 such that the nested tiers 1206, 1208, 1210, and 1212 expand to an expanded state. Additionally views and schematics are provided in FIGS. 12E and 12F. Another example of this concept is provided in the swelling structure 1300 of FIGS. 13A-13E. In FIG. 13A, the swellable material 1302 and an associated shell 1304 are shown in a pre-administration state from a side view, wherein the swellable material 1302 is a base and protruding core within the associated shell 1304 that forms the swelling structure 1300. The shell 1304 is configured to expand to a shape having at least two tiers having sequentially smaller outer dimensions. The associated shell 1304 comprises a means for water to access the swellable material 1302, e.g., a pore and/or a semi-permeable material. A top view of the swellable material 1302 and the associate shell 1304 are shown in FIG. 13B. As seen from the top view of FIG. 13B, there are four tiers 1306, 1308, 1310, and 1312 that nest together in the pre-administration state configured in a concentric arrangement. Post-administration state views of the swelling structure 1300 are provided in FIGS. 13C and 13D, wherein the swelling material 1302 has expanded to fill the associated shell 1304 such that the nested tiers 1306, 1308, 1310, and 1312 expand to an expanded state. Additionally views and schematics are provided in FIGS. 13E and 13F.

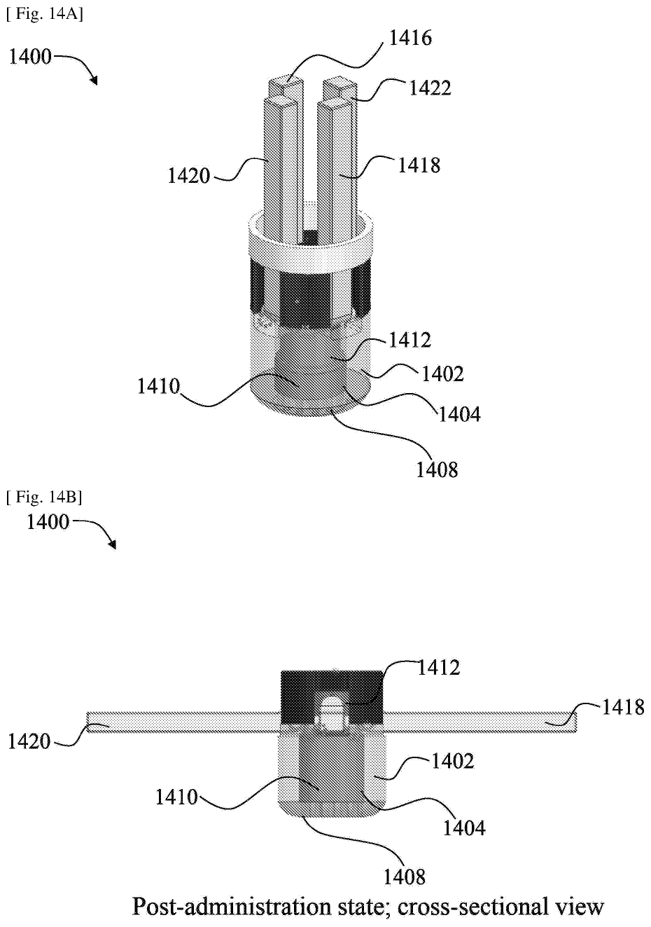

An example dosage form having four movable arms is provided in FIGS. 14A and 14B, wherein the oral drug dosage form comprises a column configured to push the moveable arms via a force provided by a swellable material. A pre-administration state of the oral drug dosage form 1400 is provided in FIG. 14A, wherein the oral drug dosage form 1400 comprises a body 1402 having a swellable material compartment 1404 configured to contain at least a portion of the swellable material 1410. The body comprises a set of fluid inlets 1408 operably connected to the swellable material compartment 1404. A column 1412 is positioned on top of the swellable material 1410. The body 1402 comprises an orifice operably connected to the swellable material compartment 1404, wherein upon swelling the swellable material 1410 pushes the column 1412 and the at least a portion of the column may extend out of and beyond the orifice. The oral drug dosage form 1400 comprises four moveable arms (the view of FIG. 14A shows four of the moveable arms 1416, 1418, 1420, 1422), each configured such that at least a portion of each moveable arm is extendible beyond, or further away from, the body of the oral drug dosage form 1400 via a force that is provided by the swellable material 1410. Specifically, each moveable arm is configured to rotate on an axis. A cross-sectional view of the oral drug dosage form 1400 is provided in FIG. 14B, wherein the swellable material 1410 has swelled and pushed the column 1412 exerting a force on each moveable arm such that each moveable arm moves to an extended position. In some embodiments, the components of the oral drug dosage form 1400 may be produced, such as via three-dimensional printing, injection molding, ultrasonic welding, or any combination thereof, and then assembled to form the pre-administration oral drug dosage form 1400. For example, in some embodiments, the body (or one or more portions thereof) is produced independently from each moveable arm, and such components are later assembled to form the oral drug dosage form.

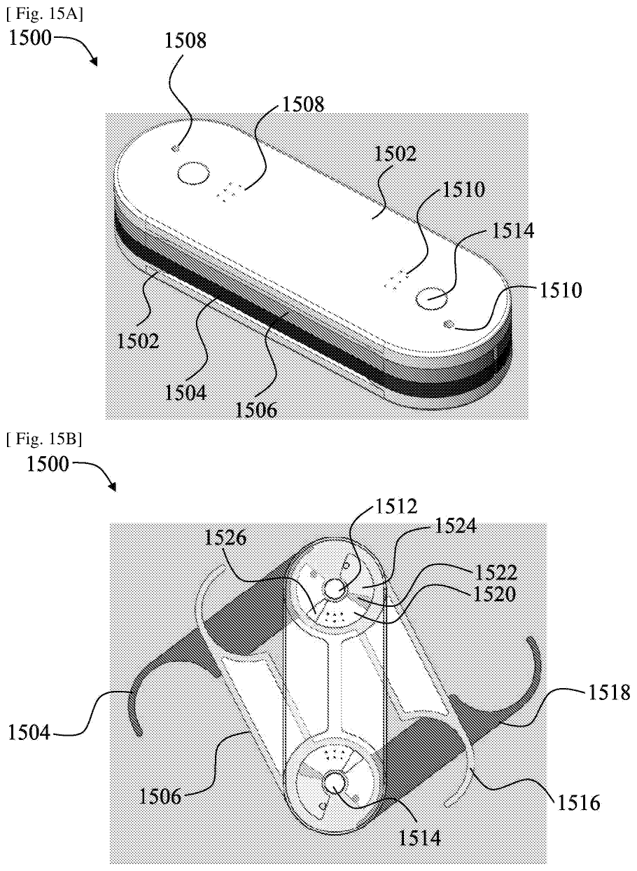

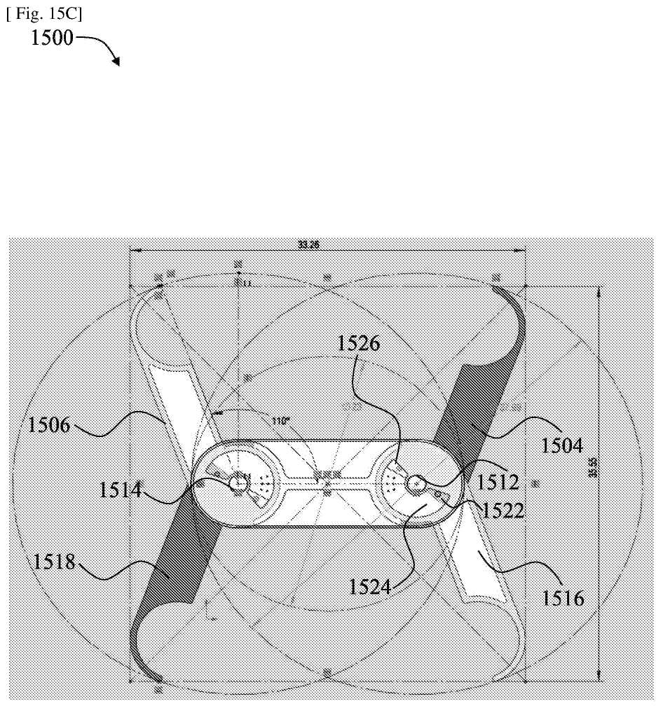

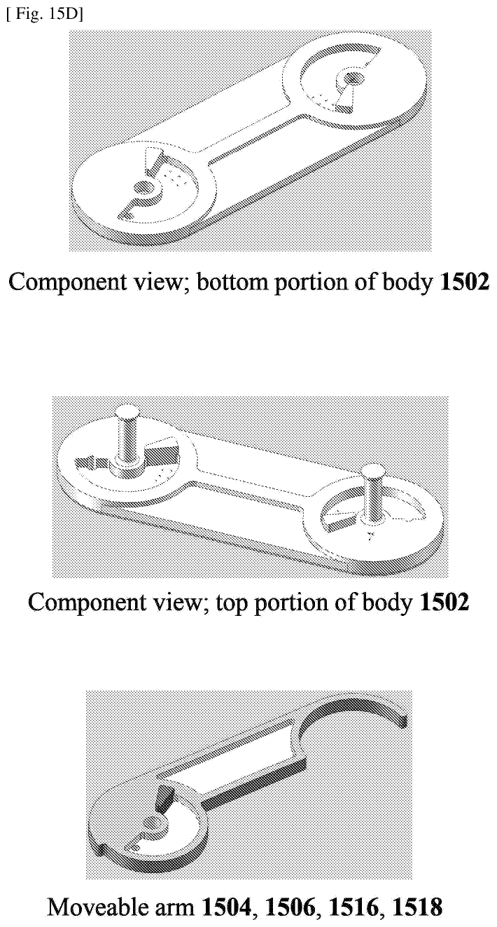

An example oral drug dosage form 1500 comprising four movable arms is illustrated in FIGS. 15A-15D. As shown in FIG. 15A, in a pre-administration state, the oral drug dosage form 1500 comprises a body 1502 forming two opposing exterior portions of the drug dosage form, wherein a first moveable arm 1504 and a second moveable arm 1506 forming are visible in the view. As shown in FIG. 15A, the body comprises a first set of fluid inlets 1508, a second send of fluid inlets 1510, a first axis, and a second axis 1514. FIG. 15B shows a post-administration state where the moveable arms 1504, 1506, 1516, and 1518 have started to extend away from the body 1502 of the oral drug dosage form 1500. Following administration, the first set of fluid inlets allow gastrointestinal fluid to enter the swellable material compartment 1520 comprising a swellable material. The third moveable arm 1516 comprises a rudder-shaped feature 1522 that sits in a direction channel 1524, wherein upon expansion of the swellable material the rudder-shaped feature 1522 is pushed through the directional channel to extend the moveable arm 1516 until a stop 1526 is reached. The first moveable arm 1504 operates using this type of mechanism and both the first moveable arm 1504 and the second moveable arm 1516 rotate on a shared axis. The second moveable arm 1506 and the fourth moveable arm 1518 operate using this type of mechanism and rotate on a second axis 1514. FIG. 15C illustrate a post-administration state of the oral drug dosage form 1500 wherein the moveable arms are fully extended. Component views of aspects of the oral drug dosage form 1500 are provided in FIG. 15D. In some embodiments, the components of the oral drug dosage form 1500 may be produced, such as via three-dimensional printing, injection molding, ultrasonic welding, or any combination thereof, and then assembled to form the pre-administration oral drug dosage form 1500. For example, in some embodiments, the body (or one or more portions thereof) is produced independently from the first moveable arm and second moveable arm, and such components are later assembled to form the oral drug dosage form.

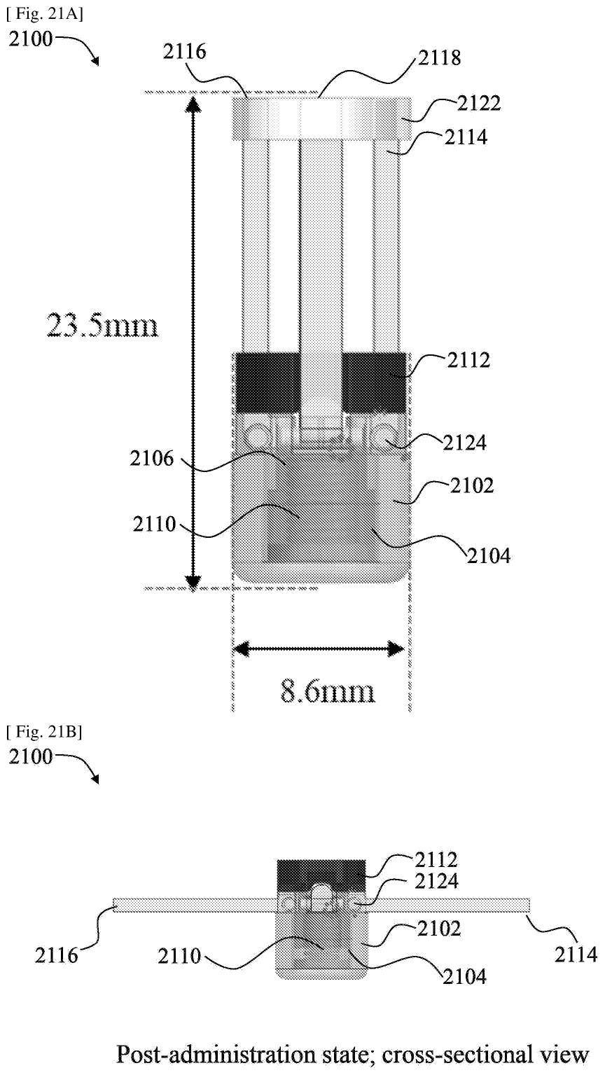

An example dosage form 2100 having four movable arms is provided in FIGS. 21A-21C. A pre-administration state of the oral drug dosage form 2100 is provided in FIG. 21A, wherein the oral drug dosage form 2100 comprises a body 2102 having an expandable material compartment 2104 configured to contain at least a portion of the expandable material 2110. Analogous to other teachings provided herein for oral drug dosage forms having a swellable material, in certain aspects an expandable material can be used as a mechanism to change the oral drug dosage form from a pre-administration state to a post-administration state. In this embodiment, the body 2102 comprises an orifice 2106 operably connected to the expandable material compartment 2104, wherein upon swelling the expandable material 2110 may extend out of and beyond the orifice 2106 (or to at least to a greater degree if the expandable material already protrudes through the orifice). The expandable material 2110 may be configured in conjunction with a non-expandable feature, such as to maintain a certain shape of at least a portion of the expandable material and/or contact a contact element of one or more moveable arms. As illustrated in FIG. 21A, the expandable material is embedded in a non-expandable feature encapsulating the expandable material in the pre-administration state (as later shown in FIG. 21C, the non-expandable feature 2128 expands with the expandable material 2110 and contacts each of the moveable arms). The body of the oral drug dosage form 2100 comprises a cap 2112. The oral drug dosage form 2100 comprises four moveable arms (the view of FIG. 21A shows three of the moveable arms 2114, 2116, 2118), each configured such that at least a portion of each moveable arm is extendible beyond, or further away from, the body of the oral drug dosage form 2100 via a force that is provided by the expandable material 2110. Specifically, each moveable arm is configured to rotate on an axis. For example, the first moveable arm 2114 is configured to rotate on a first axis 2124. As shown in a pre-administration state shown in FIG. 21A, the oral drug dosage form 2100 may further comprise an erodible restrainer 2122 configured to inhibit extension of moveable arms in a pre-administration state. A cross-sectional view of the oral drug dosage form 2100 is provided in FIG. 21B, wherein the expandable material 2110 has expanded through and beyond (at least in part) of the orifice 2106 of the body 2102 exerting a force on each moveable arm such that each moveable arm moves to an extended position. FIG. 21C shows a top view of a post-administration state of the oral drug dosage form 2100, wherein the first moveable arm 2114, the second moveable arm 2116, the third moveable arm 2118, and the fourth moveable arm 2120 are shown in an extended position. In some embodiments, the post-administration state forms a designed shape, provided by the expandable material and/or the non-expandable feature. In some embodiments, the components of the oral drug dosage form 2100 may be produced, such as via three-dimensional printing, injection molding, ultrasonic welding, or any combination thereof, and then assembled to form the pre-administration oral drug dosage form 2100. For example, in some embodiments, the body (or one or more portions thereof) is produced independently from each moveable arm, and such components are later assembled to form the oral drug dosage form.

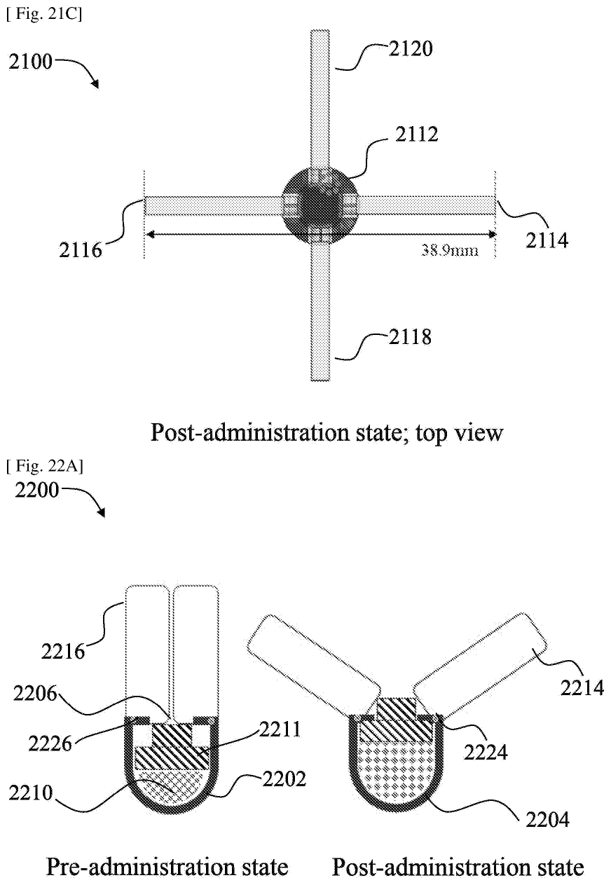

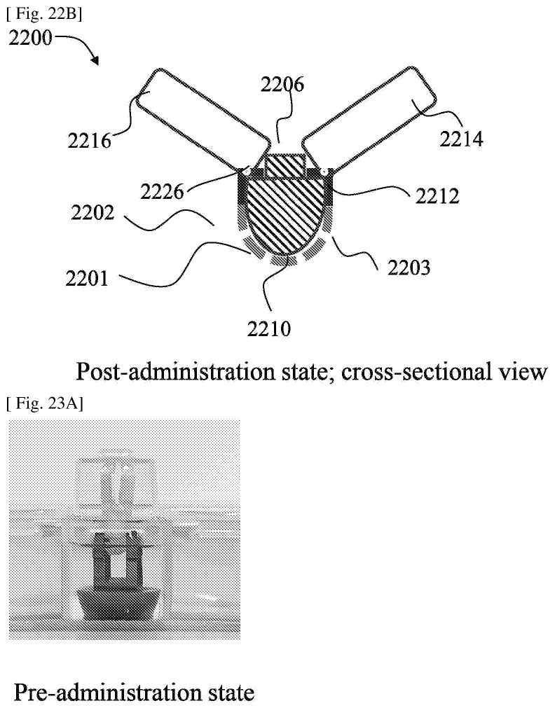

An example dosage form 2200 having movable arms is provided in FIGS. 22A-22B. In FIG. 22A, the oral drug dosage form 2200 comprises moveable arms 2114, 2116. Specifically, each moveable arm is configured to rotate on an axis. For example, the first moveable arm 2114 is configured to rotate on a first axis 2224, the second moveable arm 2116 is configured to rotate on a second axis 2226, wherein the oral drug dosage form 2200 comprises a body 2202 having an expandable material compartment 2204 configured to contain at least a portion of the expandable material 2210.

In the embodiment shown in FIG. 22A, the expandable material compartment 2204 contains expandable material 2210 and a piston 2211, wherein the piston has a certain rigidity. In the pre-administration state, the piston 2211 is located in the expandable material compartment 2204. In the post-administration state, the expandable material 2210 absorbs liquid (such as gastric or intestinal fluid) and increases in volume, pushing the piston 2211 to move towards the moveable arms 2114, 2116. The body 2202 comprises an orifice 2206 operably connected to the expandable material compartment 2204, wherein expansion of the swellable material in the presence of a gastrointestinal fluid, the piston 2211 may extend out of and beyond the orifice 2206. As illustrated in FIG. 22B, the body of the drug dosage form 2200 comprises a body 2202, where in the body comprises a cap 2212 and a base 2201, and the movable arms 2214, 2216 are connected to the cap 2212 by the pivot 2226, wherein one or more fluid inlets 2203 operably located in the base 2201. In some embodiments, the fluid inlets 2203 could be filled with soluble material. When the drug dosage form contacts with water, the soluble material begins to dissolve, and the fluid inlets is connecting to expandable material compartment 2204. Each moveable arm is extendible rotate around the body, the body of the oral drug dosage form 2200 via a force that is provided by the expandable material 2210. In some embodiments, the expandable material 2210 is pre-shaped. After absorbing water, the expandable material 2210 expands according to the preset shape until part of the expandable material 2210 protrudes from the orifice 2206 and pushes the movable arm 2214 to rotate. The expandable material moves in the direction of the orifice until it is blocked by a cap 2212. A cross-sectional view of the oral drug dosage form 2200 is provided in FIG. 22B. In some embodiments, the components of the oral drug dosage form 2200 may be produced, such as via three-dimensional printing, injection molding, ultrasonic welding, or any combination thereof, and then assembled to form the pre-administration oral drug dosage form 2200. For example, in some embodiments, the body (or one or more portions thereof) is produced independently from each moveable arm, and such components are later assembled to form the oral drug dosage form.

### B. Components of the Oral Drug Dosage Forms and Materials Thereof

In certain aspects, provided herein are components of oral drug dosage forms, such as swellable materials and/or expandable materials and associated non-swellable features, including swelling structures, one or more moveable arms, bodies and features formed therein, drugs, and erodible restrainers.

The drug dosage forms described herein contain one or more features producing a force that contributes (in whole or in part) to a change in the pre-administration state to the post-administration state of the drug dosage form. In some embodiments, the oral drug dosage form comprises a swellable material and/or an expandable material. The swellable materials described herein are configured to expand in the presence of gastrointestinal fluid and produce a force exerted on one more moveable arms, or a feature associated with the swellable material (such as a non-swellable feature covering at least a portion of the swellable material, including a non-swellable feature comprising a semi-permeable material), such that an oral drug dosage form transitions to an extended post-administration form. In some embodiments, the oral drug dosage form comprises a single unit of a swellable material. In some embodiments, the oral drug dosage form comprises two or more separate units of one or more swellable materials (e.g., a first swellable material in a first swellable material compartment and a second swellable material in a second swellable material compartment). In embodiments having two more separate units of one or more swellable materials, the swellable material of the separate units can be the same or different.

In some embodiments, the swellable material and/or the expandable material is configured to expand at a desired rate and/or with a desired force. For example, in some embodiments, the swellable material and/or the expandable material is configured to quickly expand upon contact with gastrointestinal fluid (including contact of the oral drug dosage form to the gastrointestinal fluid) to prevent an oral drug dosage form from passing through the stomach prior to remaining in the stomach for a desired stomach residency period. In some embodiments, the swellable material and/or the expandable material is configured to expand with the force necessary to move components of an oral drug dosage form to a stomach retention state, such as to move one or more moveable arms.

In some embodiments, the swellable material expands at least about 1.2×, such as at least about any of 1.3×, 1.4×, 1.5×, 1.6×, 1.7×, 1.8×, 1.9×, or 2.0×, in volume following exposure to a gastrointestinal fluid. In some embodiments, the expansion occurs within about 1 hour, such as within about any of 50 minutes, 40 minutes, 30 minutes, 20 minutes, 10 minutes, or 5 minutes. In some embodiments, the swellable material expands at least about 1.2× in 30 minutes, such as at least about any of 1.3× in 30 minutes, 1.4× in 30 minutes, 1.5× in 30 minutes, 1.6× in 30 minutes, 1.7× in 30 minutes, 1.8× in 30 minutes, 1.9× in 30 minutes, or 2.0× in 30 minutes, in volume following exposure to a gastrointestinal fluid. In some embodiments, the swellable material reaches a substantially complete swelled state (e.g., absorbs at least about 90% of its fluid capacity) within about 1 hour or less, such as within about any of 50 minutes, 40 minutes, 30 minutes, 20 minutes, 10 minutes, or 5 minutes.

In some embodiments, the swellable material, upon swelling, at least in part conforms to the shape of a direction channel and/or an orifice, or a portion thereof. In some embodiments, the swellable material, upon swelling, adopts a pre-determined shape and/or size, at least in part. In some embodiments, the pre-determined shape and/or size is guided, at least in part, by a non-swellable feature associated with a swellable material, such as a non-swellable material surround at least a portion of the swellable material in a pre-administration state. In some embodiments, the general shape of the swellable material after swelling is different than the shape of the swellable component prior to swelling. In some embodiments, the swellable material comprises a coating. In some embodiments, the coating of the swellable material delays the swelling of the swellable material, such as by inhibiting contact with gastrointestinal fluid and/or restraining swelling, for at least a pre-determined amount of time after administration of an oral drug dosage form to an individual. In some embodiments, the coating completely surrounds a swellable material. In some embodiments, the coating partially surrounds a swellable material. In some embodiments, the swellable material is completely or partially surrounded by a semi-permeable material.

In some embodiments, the swellable material comprises sodium alginate (SA), hydroxypropyl cellulose (HPC), hydroxyethyl cellulose (HEC), hydroxypropyl methyl cellulose (HPMC), polyethylene oxide (PEO), polyvinyl alcohol (PVA), microcrystalline cellulose (MCC), croscarmellose sodium (CCNa), carboxymethylcellulose sodium (CMC-Na), polyvinylpolypyrrolidone (PVPP), carboxymethyl starch sodium (CMS-Na), polyethylene glycol (PEG), or a mixture thereof. In some embodiments, HPC comprises L-HPC or H-HPC, or a combination thereof.

In some embodiments, the swellable material comprises a material selected from the group consisting of: a cross-linked product, and a shape memory material. In some embodiments, the swellable material comprises a material selected from the group consisting of: polyethylene oxide-polyethylene glycol (PEO-PEG) cross-linked polymer, polycaprolactone-polyethylene glycol-polycaprolactone (PCL-PEG-PCL), hydroxypropylcellulose, polyethylene oxide (PEO), such as high molecular weight PEO, sodium alginate, carbomer, high molecule weight hydroxypropyl cellulose (HPC), high molecular weight hydroxylpropyl methylcellulose or Hypromellose (HPMC), methyl cellulose (MC), high molecular polyvinyl alcohol (PVA), polyvinyl acetate (PVAc) and polyvinylpyrrolidone (PVP) 80/20, methacrylic ester copolymer, ammonioalkyl methacrylate copolymer, amino alkyl methacrylate copolymer E, hydroxypropyl methylcellulose acetate succinate or hypromellose acetate succinate (HPMCAS), hydroxypropyl methylcellulose phthalate (HPMCP), or combinations thereof. In some embodiments, the shape memory material is selected from the group consisting of polyurethanes, block copolymer of polyethylene terephthalate (PET) and polyethyleneoxide (PEO), block copolymers containing polystyrene and poly (1, 4-butadiene), an ABA triblock copolymer made from poly (2-methyl-2-oxazoline) and polytetrahydrofuran, polynorbornene (Norsorex, developed by CdF Chemie/Nippon Zeon), polynorbornene with partially substituted polyhedral oligosilsesquioxane (POSS), copolymer consisting of polycyclooctene (PCOE) and poly (5-norbornene-exo, exo-2, 3-dicarboxylic anhydride) (PNBEDCA), poly (ester-urethane) s, composites composed of a polyol (soft segment) and a diisocyanate coupled with a chain extender (hard phase) (poly (ε-caprolactone) (PCL), poly (ethylene adipate) (PEA) glycol)), the combination of poly (ester-urethane) s (PURs) and PCL, ethylene oxide-ethylene terephthalate segmented copolymers, oligo (ε-caprolactone)- and oligo (p-dioxanone)-based PUR, poly (p-dioxanone)-b-poly (tetramethylene oxide glycol) multiblocky copolymers, poly (methyl methacrylate)-poly (ethylene glycol) (PMMA-PEG) semi-interpenetrated network (IPN), poly (cyclohexylmethacrylate) (PCHMA) main-chain cross-linked with difunctional PCL polymers, polymers with grafted short PEG side chains on the PCL main chains, Nafion®, copoly (ester-urethane) network, covalently cross-linked poly [ethylene-co-(vinyl acetate)] (cPEVA), combination of PCL and poly (tetramethylene ether) glycol (PTMEG), or combinations thereof. In some embodiments, the material may have a plurality of properties, including a swellable material and a shape memory material.

In some embodiments, the swellable material further comprises a salt or a mixture of salts, e.g., to promote absorption of gastrointestinal fluids to promote swelling. In some embodiments, the salt is selected from the group consisting of a sodium salt, a magnesium salt, and a kali salt. In some embodiments, the sodium salt is Na₂SO₄.

In some embodiments, the swellable material further comprises a gas-producing substance, for example, by contacting with a gastrointestinal fluid to produce carbon dioxide, in some embodiments, the gas-producing substance is selected from carbonate, bicarbonate, or a combination thereof.

In some embodiments, the expandable material comprises a material selected from the group consisting of: poly (vinyl acetate) (PVAc), polyvidone, hydroxypropyl methyl cellulose phthalate, methacrylic acid copolymer, ethyl cellulose (EC), hydroxypropyl methyl cellulose (HPMC), tocopherol polyethylene glycol succinate (TPGS), polycaprolactone (PCL), polyethylene (PE), guar, poly-ether-ether-ketone (PEEK), polyphenylsulphone (PPSU), polysulfone (PSU), polypropylene (PP), ethylene vinyl acetate (EVA), polymethyl methacrylate (PMMA), polylactic acid (PLA), polyglycolideacid (PGA), poly (lactic-co-glycolic acid) (PLGA), or combinations thereof.

In some embodiments, the swellable material does not contain a drug.

In some embodiments, the swellable material in the swellable material compartment is of an amount of at least about 5 mg, such as at least about any of 10 mg, 20 mg, 30 mg, 40 mg, 50 mg, 75 mg, 100 mg, 125 mg, 150 mg, 175 mg, or 200 mg.

In some embodiments, the swellable material has a swelling volume of about 10 mm³ to about 50 mm³. In some embodiments, the swellable material has a swelling volume of at least about 5 mm³, such as at least about any of 10 mm³, 15 mm³, 20 mm³, 25 mm³, 30 mm³, 35 mm³, 40 mm³, 45 mm³, or 50 mm³.

In some embodiments, the swellable material can be printed via a three-dimensional printing process, injection molding, ultrasonic welding, or any combination thereof, such as described in other aspects of the present disclosure. In some embodiments, the swellable material is a thermoformable material.

In some embodiments, the body comprises two or more pieces configured to form the body. In some embodiments, the body comprises one or more bases and one or more caps. In some embodiments, the body comprises two or more materials. In some embodiments, the body of the oral drug dosage form is a monolithic structure. In some embodiments, the body delineates the outer bounds of the oral drug dosage form in a pre-administration state. In some embodiments, the body can be printed via a three-dimensional printing process, injection molding, ultrasonic welding, or any combination thereof, such as described in other aspects of the present disclosure. In some embodiments, the body is composed of a thermoformable material.

In some embodiments, the body comprises one or more swellable material compartments, such as any of 1, 2, 3, 4, 5, 6, 7, 8, 9, or 10 swellable material compartments. In some embodiments, the swellable material compartment is configured to substantially contain the swellable material in a pre-administration state of an oral drug dosage form. In some embodiments, the swellable material compartment and a contact element of a moveable arm substantially surround the swellable material in a pre-administration state of an oral drug dosage form. The swellable material compartment may be any size and/or shape.

In some embodiments, the body comprises one or more directional channels and/or orifices. In some embodiments, the directional channel is configured to direct movement of a moveable arm via a force provided by a swellable material. Directional channels and/or orifices may be any shape and/or size, and, as taught herein, such shape and/or size may be guided by other design features of an oral drug dosage form, e.g., the mechanism and shape of a moveable arm and amount of swellable material needed to obtain a post-administration state for gastric retention. In some embodiments, the directional channel comprises a curved-shaped channel. In some embodiments, the directional channel comprises a circular-shaped channel. In some embodiments, the directional channel comprises a square or rectangular-shaped channel. In some embodiments, the orifice is square or circular or rectangular-shaped.

In some embodiments, the directional channel further comprises one or more fluid inlet (such as described below) configured to further expose a swellable material to gastrointestinal fluid during the swelling process.

In some embodiments, the body comprises one or more fluid inlets, such as any of about 5 to about 20, per swellable material compartment, wherein the one or more fluid inlets are operably connected to the swellable material compartment. In some embodiments, at least one of the one or more fluid inlets comprises a semi-permeable material. In some embodiments, the at least one of the one or more fluid inlets is the semi-permeable material filling a pore formed by the body. In some embodiments, the one or more fluid inlets is a semi-permeable membrane at least partially wrapping around the swellable material. In some embodiments, at least one of the one or more fluid inlets is a pore. In some embodiments, the one or more fluid inlets is a pore having a largest crossing dimension (such as a diameter) of about 0.5 to about 1 mm. The pore may be any shape, including circular, square, or rectangular.

In some embodiments, the body further forms a stop configured to engage with the moveable arm at an extended position. The stop may be any portion of the body and need not have a specific design. In some embodiments, the stop comprises a locking element configured to maintain the moveable arm at one or more extended positions.

As described herein, the oral drug dosage forms comprise one or more moveable arms, such as any of 1, 2, 3, 4, 5, 6, 7, 8, or 10 moveable arms. The moveable arms may be designed using a number of mechanisms based on swelling of a swellable material, and an oral drug dosage form may implement one or more such mechanisms. In some embodiments, the moveable arm, or a portion thereof, is configured to slide within a directional channel. In some embodiments, the contact element of a moveable arm is configured to slide within a directional channel. In some embodiments, the moveable arm is configured to rotate on an axis. In some embodiments, at least two of the moveable arms or an oral drug dosage form have a different movement mechanism. In some embodiments, at least two of the moveable arms of an oral drug dosage form have the same movement mechanism. In some embodiments, at least two of the moveable arms of an oral drug dosage form are configured such that at least a portion of each of the moveable arms is extendible beyond, or further away from, the body of the oral drug dosage form via a force that is provided by the swellable material In some embodiments, one or more moveable arms is associated with a single swellable material in a swellable material compartment For example, swellable material in a swellable material compartment swells and leads to actuation of one or more moveable arms. In some embodiments, the oral drug dosage form comprises two moveable arms, wherein the first moveable arm is associated with a first swellable material in a first swellable material compartment, and a first moveable arm is associated with a second swellable material in a second swellable material compartment.

In some embodiments, the oral drug dosage form further comprises an erodible restrainer configured to inhibit extension of one or more moveable arm for a duration of time. Generally speaking, the erodible restrainer is configured to facilitate oral administration of an oral drug dosage form, e.g., by preventing early extension of one or more moveable arms prior to entering the stomach of an individual. In some embodiments, the erodible restrainer erodes within about 30 minutes, such as within about any of 25 minutes, 20 minutes, 15 minutes, 10 minutes, or 5 minutes, following administration to an individual.

In some embodiments, certain components of an oral drug dosage form, such as a body or an moveable arm, comprises an insoluble material, a pH-sensitive erosion material, e.g., a material that does not erode in the pH of the stomach, a slowly eroding material, e.g., a material that will erode after the oral drug dosage form, or components thereof, leave the stomach, or a combination thereof. In some embodiments, certain component, such as a body or an moveable arm, comprises a material selected from the group consisting of: ammonio methacrylate copolymer, ammonio-methacrylate copolymer type B, stearic acid, ethyl cellulose (EC), titanium dioxide, cellulose acetate phthalate (CAP), poly (lactide-co-glycolide) (PLGA), ethylene-vinyl acetate copolymer, polyethylene (PE), polycaprolactone (PCL), polylactic acid (PLA), cellulose acetate butyrate (CAB), cellulose acetate (CA), polyvinyl acetate (PVAc), polyvinyl acetal diethyl amino lactate (AEA), poly (butyl methacrylate-co-(2-dimethylaminoethyl) methacrylate-co-methyl methacrylate), and poly (ethyl acrylate, methyl methacrylate, trimethyl aminoethyl methacrylate chloride), or a combination thereof.

In some embodiments, the material of components, such as a body or an moveable arm, of an drug dosage form is a thermoplastic material. In some embodiments, the thermoplastic material is a thermoplastic polymer. In some embodiments, the thermoplastic material comprises any one or more of a plasticizer, and another additive, e.g., a filler, a binder, a lubricant, a glidant, and a disintegrant. In some embodiments, the additive is selected from the group consisting of a clay, a SiC nanoparticle, a Ni powder, a carbon nanotube, a carbon fiber, a carbon black, a graphene, a metal oxide (e.g., Fe₃O₄, TiO₂, ZnO), a silver (Ag) nanoparticle, a gold (Au) nanoparticle, a silver and gold nanoparticle, a nanorod, a nanowhisker, a nanowire, and a cellulose nanocrystals.

In some embodiments, the body is configured to have wall thicknesses (such as from a swellable material compartment to an external portion of an oral drug dosage form or from a directional channel to an external portion of the oral drug dosage form) of at least about 0.3 mm, such as at least about 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm, 1.9 mm, 2.0 mm, 2.1 mm, 2.2 mm, 2.3 mm, 2.4 mm, or 2.5 mm. In some embodiments, the body is configured to have wall thicknesses (such as from a swellable material compartment to an external portion of an oral drug dosage form or from a directional channel to an external portion of the oral drug dosage form) of about 0.3 mm to about 2.5 mm, such as any of about 0.5 mm to about 2.2 mm or about 0.4 mm to about 1.2 mm.

In some embodiments, the arm is configured to have thickness of about 1 mm to about 2 mm, such as about 1.1 mm to about 1.6 mm.

In some embodiments, the oral drug dosage form comprises a component, the component is a plunger or a piston, such as a column, configured to be pushed by a swellable material into one or more arms. In some embodiments, the column comprises a wall thickness of about 0.3 mm to about 2.5 mm, such as about 0.4 mm to about 2.2 mm. In some embodiments, the column comprises a wall thickness of at least about 0.3 mm, such as at least about any of 0.4 mm, 0.5 mm, 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm. 1.9 mm, 2.0 mm, 2.1 mm, 2.2 mm, 2.3 mm, 2.4 mm, or 2.5 mm.

### C. Integration of One or More Drugs in the Oral Drug Dosage Forms and Drug Release Profiles

The oral drug dosage forms described herein comprise one or more drugs. In some embodiments, the oral drug dosage form comprises a drug, including separate portions of the drug formulated and configured with substantially matching or different release profiles. In some embodiments, the oral drug dosage form comprises 2 or more drugs, such as any of 3, 4, or 5 drugs. The drug or drugs of the oral drug dosage forms can be released at any point of the life cycle of the oral drug dosage form. For example, in some embodiments, the oral drug dosage form is configured and formulated to release a drug in the stomach of an individual. In some embodiments, substantially all, such as at least about any of 70%, 75%, 80%, 85%, 90%, 95%, or 100%, of the drug in an oral drug dosage form is released in the stomach. In some embodiments, the oral drug dosage form is configured to release a drug prior to or during swelling of a swellable material, such as an immediate-release profile. In some embodiments, the drug dosage form is configured to release a drug after leaving the stomach.

In some embodiments, the drug is a poorly-water soluble drug. In some embodiments, the poorly-water soluble drug is a Biopharmaceutics Classification System (BCS) Class II active pharmaceutical ingredient (API), e.g., a drug having high permeability and low solubility. In some embodiments, the poorly-water soluble drug is a Biopharmaceutics Classification System (BCS) Class IV active pharmaceutical ingredient (API), e.g., a drug having low permeability and low solubility. In some embodiments, the drug is a Biopharmaceutics Classification System (BCS) Class III active pharmaceutical ingredient (API), e.g., a drug having low permeability and high solubility. In some embodiments, the drug is selected from the group consisting of riociguat, aceclofenac, bicalutamide, carbamazepine, carvedilol, clotrimazole, cinnarizine, danazol, dapsone, estradiol, exetimibe glibenclamide, fenofibrate, griseofulvin, ibuprofen, itraconazole, ketoconazole, mefenamic acid, naproxen, nevirapine, nifedipine, nitrofurantoin, nomegestrol acetate, phenytoin sodium salt, piroxicam, praziquantel, rifampicin, sulfamethoxazole, trimethoprim, and verapramil hydrochloride.

The oral drug dosage forms may be configured to release a drug based on any desired release profile, and extends to release more than one drug wherein each drug has any desired release profile. Generally, as the oral drug dosage forms described herein are designed to be retained in the stomach for an extended period of time, the release profile of at least one drug will be configured based on the expected stomach residency of the oral drug dosage form. In some embodiments, when two or more drugs are in the oral drug dosage form, the oral drug dosage form is configured to release each drug according to a desired release profile. In some embodiments, the oral drug dosage form is configured such that all drug content (or substantially all, such as at least about 90%) in the oral drug dosage form (or a portion thereof) is released during the expected stomach residency of the oral drug dosage form. In some embodiments, the oral drug dosage form is configured such that an amount of the drug content (such as a second drug) in the oral drug dosage form is released after the oral drug dosage form, or components thereof, are expected to be expelled from the stomach. In some embodiments, the oral drug dosage form is formulated and configured such that a drug is released according to a delayed release profile, a sustained release profile, a delayed-sustained release profile, a zero-order release profile, a first-order release profile, an immediate release profile plus a sustained release profile, an immediate release profile plus a delayed release profile, an immediate release profile plus a delayed-sustained release profile, a pulsed release profile, an iterative pulsed release profile, an immediate release profile plus a pulsed release profile, or a combination thereof.

The oral drug dosage forms described herein may be configured and formulated to release a drug according to a desired drug release profile using a variety of techniques. In some embodiments, the release of a drug from an oral drug dosage form is based on the erosion of a drug-containing material, such as when the drug-containing material is exposed to gastrointestinal fluid. In some embodiments, the drug-containing material is configured as a layer having a pre-determined surface area, such a surface area exposed to the gastrointestinal fluid, thickness, and drug mass fraction, wherein these characteristics of the drug-containing material provide a desired drug release. In some embodiments, the drug-containing material is in the form of a multi-layered structure. In some embodiments, the drug-containing material is embedded, including partially embedded, in a material of a component of a drug dosage form. Design, configurations, and materials of such drug-contain materials to provide a desired drug release are known in the art, e.g., see U.S. Pat. No. 10,350,822, which is hereby incorporated herein in its entirety.

In some embodiments, the oral drug dosage form is configured with a drug-containing compartment, wherein the compartment has an orifice from which drug is release from the oral drug dosage form. In some embodiments, the orifice is blocked with an erodible material, such as a plug. In some embodiments, the feature blocking the orifice, such that a drug is held within a compartment of an oral drug dosage form, is configured to no longer block the orifice at a desired time. For example, in some embodiments, the drug-containing compartment is sealed with an erodible plug, wherein the erodible plug dissolves at a certain time after administration to the individual to thereby release the drug from an oral drug dosage form. The timing of release can be based on, e.g., thickness of the plug and/or material of the plug. The drug-containing compartment can be configured in any component. In some embodiments, the oral drug dosage form comprises a plurality of drug-containing compartments. In some embodiments, the component is a non-erodible material, such as an insoluble shell material. In some embodiments, the component erodes after the drug has left the drug-containing compartment, e.g., after an oral drug dosage form exits the stomach.

In some embodiments, the oral drug dosage form is configured such that a drug will leach or diffuse from a material.

In some embodiments, the drug is loaded on one or more of the movable arms; In some embodiments, each movable arm is loaded with drug. In some embodiments, the drug is loaded on one or more of the caps. In some embodiments, the drug is loaded in the swellable material compartment.

The drug or drugs of the drug dosage forms described herein may be a part of any component described herein. In some embodiments, the drug is not located in a swellable material In some embodiments, the swellable material does not comprise a drug.

### D. Characteristics of the Oral Drug Dosage Forms in a Post-Administration State to Provide Gastric Retention

As described herein, the swelling of one or more swellable materials, or portions thereof, increases the dimension(s) of an oral drug dosage form, thereby allowing the drug to be retained in the stomach for an extended period of time. In some embodiments, the post-administration state of an oral drug dosage form is referred to as a stomach retention state. One of ordinary skill in the art will readily appreciate that characteristics of the oral drug dosage form, such as size, during the swelling of the swellable material, or a portion thereof, may be dynamic and change over time. The description of certain states of the oral drug dosage form, such as a post-administration stomach retention state, is not intended to limit the disclosure herein to only that one static embodiment of the drug dosage form.

In some embodiments, when the oral drug dosage form is in an expanded state (e.g., the stomach retention state), the oral drug dosage form is a size that inhibits and/or prevents passage of the oral drug dosage form through an aspect of the pylorus (such as the pyloric antrum, the pyloric canal, or the pyloric orifice create by the pyloric sphincter) to the duodenum. In some embodiments, when the oral drug dosage form is in an expanded state, the oral drug dosage form is a size that inhibits and/or prevents passage of the oral drug dosage form through the pyloric orifice created by the pyloric sphincter. In some embodiments, when the oral drug dosage form is in an expanded state, at least two perpendicular dimensions of the oral drug dosage form are each independently at least about 20 mm to about 70 mm in length, such as at least about 20 mm to about 50 mm in length, about 30 mm to about 60 mm in length, or about 40 mm to about 70 mm in length. In some embodiments, when the drug dosage form is in an expanded state, at least two perpendicular dimensions of the drug dosage form are each independently at least about 20 mm, such as at least about any of 25 mm, 30 mm, 35 mm, 40 mm, 45 mm, 50 mm, 55 mm, 60 mm, 65 mm, or 70 mm, in length. In some embodiments, when the oral drug dosage form is in an expanded state, at least two perpendicular dimensions of the drug dosage form are each independently about any of 20 mm, 25 mm, 30 mm, 35 mm, 40 mm, 45 mm, 50 mm, 55 mm, 60 mm, 65 mm, or 70 mm, in length. In some embodiments, when the oral drug dosage form is in an expanded state, one dimension of the at least two perpendicular dimensions is different from another dimension. In some embodiments, when the drug dosage form is in an expanded state, one dimension of the at least two perpendicular dimensions is the same as another dimension. It is noted that individuals may have different anatomical characteristics and sizes (e.g., adult versus child), and the present application encompasses oral drug dosage forms designed in view of such considerations to achieve the desired gastric retention.

The oral drug dosage forms described herein are configured to be retained in the stomach for an extended period of time, such as compared to a drug dosage form without a gastric retention feature (e.g., an oral drug dosage form that passes through the stomach according to the natural flow of ingested material out of the stomach). In some embodiments, the oral drug dosage form is configured to be retained in the stomach for about 8 hours to about 3 months, such as any of about 8 hours to about 24 hours, about 8 hours to about 36 hours, about 18 hours to about 30 hours, about 20 hours to about 28 hours, about 12 hours to about 36 hours, about 1 day to about 3 days, about 3 days to about 7 days, about 8 hours to about 1 month, or about 8 hours to about 2 months. In some embodiments, the oral drug dosage form is configured to be retained in the stomach for at least about 8 hours, such as at least about any of 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, or 36 hours. In some embodiments, the oral drug dosage form is configured to be retained in the stomach for at least about 1 day, such as at least about any of 2 days, 3 days, 4 days, 5 days, 6 days, 7 days, 14 days, 21 days, 1 month, 1.5 months, 2 months, 2.5 months, or 3 months. In some embodiments, the oral drug dosage form is configured to be retained in the stomach for no longer than about 7 days, such as no longer than about any of 6 days, 5 days, 4 days, 3 days, 2 days, 36 hours, 30 hours, 24 hours, 18 hours, or 12 hours. In some embodiments, the oral drug dosage form is configured to be retained in the stomach for no longer than about 3 months, such as no longer than about any of 2.5 months, 2 months, 1.5 months, 1 month, 21 days, 14 days, or 7 days. In some embodiments, the oral drug dosage form is configured to be retained in the stomach for at about any of 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 2 days, 3 days, 4 days, 5 days, 6 days, 7 days, 14 days, 21 days, 1 months, 1.5 months, 2 months, 2.5 months, or 3 months.

In some embodiments, the post-administration state of an oral drug dosage form occurs within about 1 hour or less following administration of the oral drug dosage form to the individual. For example, in some embodiments, the oral drug dosage form obtains a post-administration stomach retention state within about any of 1 hour or less, 50 minutes or less, 40 minutes or less, 30 minutes or less, 20 minutes or less, 10 minutes or less, or 5 minutes or less of being administered to an individual.

In some embodiments, the oral drug dosage form is configured such that the oral drug dosage form, or parts thereof, can pass through the pylorus and be expelled from the stomach. For example, in some embodiments, one or more components of an oral drug dosage form, such as one or more moveable arms, erodes and/or softens in the stomach thereby allowing passage out of the stomach in one or more parts. In some embodiments, the oral drug dosage form will become softer after a period of gastric retention and is configured to pass through the pylorus as a whole. In some embodiments, the erosion or dissolution of a component, or a portion thereof, of an oral drug dosage form is due to prolonged exposure to gastrointestinal fluids in the stomach (e.g., due to prolonged exposure to a low pH).

### E. Characteristics of the Oral Drug Dosage Form in a Pre-Administration State

The oral drug dosage forms described herein may be formed in any number of shapes, sizes, weights, and appearances. As described herein, the oral drug dosage forms of the present application may take forms having different features (such as a size and shape) during the life cycle of the administered oral drug dosage form (e.g., the administration state and the post-administration stomach retention state).

In some embodiments, the oral drug dosage form described herein is suitable for oral administration to a human individual. Such oral drug dosage forms of the present application can be, for example, any size, shape, or weight that is suitable for oral administration to specific human individuals, such as children and adults. In some embodiments, the oral drug dosage form is suitable for oral administration to an individual, wherein selection of size, shape, or weight of the drug dosage form is based on an attribute of the individual, e.g., one or more of height, weight, age, or a size of an anatomical feature, such as those relevant to oral administration.

In some embodiments, the surface, such as an exterior surface, e.g., the body, of the oral drug dosage form has the shape of a capsule, circle, oval, bullet shape, arrow head shape, triangle, arced triangle, square, arced square, rectangle, arced rectangle, diamond, pentagon, hexagon, octagon, half moon, almond, or a combination thereof.

In some embodiments, the oral drug dosage form has a largest crossing dimension of about 5 mm to about 26 mm, such as any of about 5 mm to about 15 mm, about 6 mm to about 13 mm, or about 7 to about 11 mm. In some embodiments, the oral drug dosage form has a largest crossing dimension of at least about 5 mm, such as at least about any of 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21, mm, 22 mm, 23 mm, or 24 mm. In some embodiments, the drug dosage form has a largest crossing dimension of less than about 26 mm, such as less than about any of 25 mm, 24 mm, 23 mm, 22 mm, 21 mm, 20 mm, 19 mm, 18 mm, 17 mm, 16 mm, 15 mm, 14 mm, 13 mm, 12 mm, 11 mm, 10 mm, 9 mm, 8 mm, 7 mm, 6 mm, or 5 mm. In some embodiments, the drug dosage form has a largest crossing dimension of about any of 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 20.5 mm, 21 mm, 21.5 mm, 22 mm, 22.5 mm, 23 mm, 23.5 mm, or 24 mm. In some embodiments, the largest crossing dimension is measured across a surface, such as an exterior surface, of the drug dosage form (such as represented by length or width of the oral drug dosage form). In some embodiments, the largest crossing dimension is measured crossing through or in a diagonal across an oral drug dosage form.

In some embodiments, the oral drug dosage form has a crossing dimension perpendicular to the largest crossing dimension of about 5 mm to about 20 mm, such as any of about 5 mm to about 15 mm, about 6 mm to about 13 mm, or about 7 to about 11 mm. In some embodiments, the oral drug dosage form has a crossing dimension perpendicular to the largest crossing dimension of at least about 5 mm, such as at least about any of 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm. In some embodiments, the oral drug dosage form has a crossing dimension perpendicular to the largest crossing dimension of less than about 20 mm, such as less than about any of 19 mm, 18 mm, 17 mm, 16 mm, 15 mm, 14 mm, 13 mm, 12 mm, 11 mm, 10 mm, 9 mm, 8 mm, 7 mm, 6 mm, or 5 mm. In some embodiments, the oral drug dosage form has a crossing dimension perpendicular to the largest crossing dimension of about any of 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm. In some embodiments, the crossing dimension perpendicular to a largest crossing dimension is measured across a surface of an oral drug dosage form. In some embodiments, the crossing dimension perpendicular to a largest crossing dimension is measured crossing through or in a diagonal across an oral drug dosage form.

In some embodiments, the oral drug dosage form has a thickness of about 5 mm to about 20 mm, such as any of about 5 mm to about 15 mm, about 6 mm to about 13 mm, or about 7 to about 11 mm. In some embodiments, the oral drug dosage form has a thickness of at least about 5 mm, such as at least about any of 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm. In some embodiments, the oral drug dosage form has a thickness of less than about 20 mm, such as less than about any of 19 mm, 18 mm, 17 mm, 16 mm, 15 mm, 14 mm, 13 mm, 12 mm, 11 mm, 10 mm, 9 mm, 8 mm, 7 mm, 6 mm, or 5 mm. In some embodiments, the oral drug dosage form has a thickness of about any of 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm.

In some embodiments, the oral drug dosage form has a total weight of about 50 mg to about 1,000 mg, such as any of about 50 mg to about 100 mg, about 100 to about 200 mg, about 200 mg to about 300 mg, about 300 mg to about 400 mg, about 400 mg to about 500 mg, about 500 mg to about 600 mg, about 600 mg to about 700 mg, about 700 mg to about 800 mg, about 800 mg to about 900 mg, or about 900 mg to about 1,000 mg. In some embodiments, the oral drug dosage form has a total weight of at least about 50 mg, such as at least about any of 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, or 1,000 mg. In some embodiments, the oral drug dosage form has a total weight of less than about 1,000 mg, such as less than about an of 950 mg, 900 mg, 850 mg, 800 mg, 750 mg, 700 mg, 650 mg, 600 mg, 550 mg, 500 mg, 475 mg, 450 mg, 425 mg, 400 mg, 375 mg, 350 mg, 325 mg, 300 mg, 275 mg, 250 mg, 225 mg, 200 mg, 175 mg, 150 mg, 125 mg, 100 mg, 75 mg, or 50 mg. In some embodiments, the oral drug dosage form has a total weight of about any of 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, or 1,000 mg.

### F. Additional Features of the Drug Dosage Forms

In some embodiments, the oral drug dosage form comprises a gas-filled compartment, e.g., to provide buoyancy to an oral drug dosage form. In some embodiments, the gas-filled compartment is embedded in a component of the oral drug dosage form, such as a body or one or more moveable arms. In some embodiments, the gas-filled compartment has an erodible plug, wherein the erodible plug is configured to open the gas-filled compartment at a point after administration of the oral drug dosage form to the individual.

In some embodiments, the oral drug dosage form comprises an additional feature, such as an outer coating, an outer layer (such as a capsule shell), or an outer marking. In some embodiments, the outer coating or layer comprises/is a flavor coating. In some embodiments, the outer coating or layer comprises/is a sugar coating. In some embodiments, the outer coating or layer comprises/is a cosmetic coating. In some embodiments, the outer coating or layer comprises/is a color coating. In some embodiments, the outer coating or layer is a film coating. In some embodiments, the outer coating or layer is a polymer coating. In some embodiments, the outer coating completely surrounds the drug dosage form. In some embodiments, the outer layer forms a portion of the exterior of an oral drug dosage form. In some embodiments, the additional component is a label, such as a drug logo, company name or abbreviation, graphic, medication label, drug chemical name or abbreviation, drug specification, an identification barcode, or a combination thereof.

G. Exemplary medical devices The above invention concept can be applied to medical devices. In some embodiments, a medical device for gastric retention is provided, the medical device comprising: an expandable material; The movable arm is configured so that at least part of the movable arm may extend or away from the oral drug dosage form by force provided by the expandable material; The body comprises: an expandable material chamber, configured to comprise at least a portion of the expandable material; operably connected to a directional channel of the expandable material chamber, wherein the movable arm comprises a contact element configured to be close to the directional channel with the expandable material, wherein the contact element of the expandable material chamber and the movable arm substantially surrounds the expandable material, and wherein the movable arm or a portion thereof, is configured to slide within the directional channel such that the movable arm extends along an axis based on the directional channel beyond or rotating around from the body of the medical device; one or more fluid inlets, such as orifices or semi-permeable materials, operationally connected to an expandable material chamber; and drugs, wherein the medical device is configured to have a dense form in the pre-taking state and having a post-taking state with an extended form providing gastric retention, and wherein the extended form of the post-dose state of the medical device is due to, at least partially, a substance that expands in the presence of gastrointestinal fluid. In some embodiments, the body comprises a matrix and a cap. In some embodiments, the swelling material comprises a plunger.

### H. Exemplary Oral Drug Dosage Forms

In some embodiments, provided is an oral drug dosage form configured for a gastric retention, the oral drug dosage form comprising: a swellable material; a moveable arm configured such that at least a portion of the moveable arm is extendible beyond, or further away from, a body of the oral drug dosage form via a force that is provided by the swellable material; the body comprising: a swellable material compartment configured to contain at least a portion of the swellable material; a directional channel operably connected to the swellable material compartment, wherein the moveable arm comprises a contact element configured to interface with the swellable material in proximity to the directional channel, wherein the swellable material compartment and the contact element of the moveable arm substantially surround the swellable material, and wherein the moveable arm, or a portion thereof, is configured to slide within the directional channel such that the moveable arm extends beyond, or further away from, the body of the oral drug dosage form along an axis based on the directional channel; one or more fluid inlets, such as a semi-permeable material, operably connected to the swellable material compartment; a stop, or one or more stops, configured to engage with the moveable arm at an extended position; and a drug, wherein the oral drug dosage form is configured to have a pre-administration state having a compact form and a post-administration state having an extended form providing the gastric retention, and wherein the extended form of the post-administration state of the oral drug dosage form is due to, at least in part, expansion of the swellable material in the presence of a gastrointestinal fluid. In some embodiments, the stop comprises a locking element configured to maintain the moveable arm at one or more extended positions. In some embodiments, the locking element is configured to maintain the moveable arm at a single position. In some embodiments, the locking element is configured to maintain the moveable arm at a plurality of progressing positions.

In some embodiments, provided is an oral drug dosage form configured for a gastric retention, the oral drug dosage form comprising: a swellable material; a first moveable arm and a second movable arm, wherein the first moveable arm and the second moveable arm are configured such that at least a portion of each moveable arm is extendible beyond, or further away from, a body of the oral drug dosage form via a force that is provided by the swellable material, and wherein the first moveable arm and the second moveable arm extend in substantially opposite directions along an axis based on the directional channel; the body comprising: a swellable material compartment configured to contain at least a portion of the swellable material; a first directional channel and a second directional channel operably connected to the swellable material compartment, wherein the first moveable arm comprises a first contact element configured to interface with the swellable material in proximity to the first directional channel, wherein the second moveable arm comprises a second contact element configured to interface with the swellable material in proximity to the second directional channel, wherein the swellable material compartment and the first contact element of the first moveable arm and second contact element of the second moveable arm substantially surround the swellable material, and wherein the first moveable arm, or a portion thereof, is configured to slide within the first directional channel such that the first moveable arm extends beyond, or further away from, the body of the oral drug dosage form along the axis, and wherein the second moveable arm, or a portion thereof, is configured to slide within the second directional channel such that the second moveable arm extends beyond, or further away from, the body of the oral drug dosage form along the axis; one or more fluid inlets operably connected to the swellable material compartment; a first stop configured to engage with the first moveable arm at an extended position; a second stop configured to engage with the second moveable arm at an extended position; and a drug, wherein the oral drug dosage form is configured to have a pre-administration state having a compact form and a post-administration state having an extended form providing the gastric retention, and wherein the extended form of the post-administration state of the oral drug dosage form is due to, at least in part, expansion of the swellable material in the presence of a gastrointestinal fluid.

In some embodiments, provided is an oral drug dosage form configured for a gastric retention, the oral drug dosage form comprising: a first swellable material; a first moveable arm, wherein the first moveable arm is configured such that at least a portion of the moveable arm is extendible beyond, or further away from, a body of the oral drug dosage form via a force that is provided by the first swellable material, and wherein the first moveable arm is configured to rotate on a first axis; a second swellable material; a second moveable arm, wherein the second moveable arm is configured such that at least a portion of the moveable arm is extendible beyond, or further away from, the body of the oral drug dosage form via a force that is provided by the second swellable material, and wherein the second moveable arm is configured to rotate on a second axis; the body comprising: a first swellable material compartment configured to contain at least a portion of the first swellable material; a first directional channel operably connected to the first swellable material compartment, wherein the first direction channel comprises a curved-shaped channel, wherein the first moveable arm comprises a first contact element configured to interface with the first swellable material in proximity to the first directional channel, wherein the first swellable material compartment and the first contact element of the first moveable arm substantially surround the first swellable material, and wherein the first moveable arm, or a portion thereof, is configured to rotate on the first axis by way of the first contact element sliding in the first direction channel such that the first moveable arm extends beyond, or further away from, the body of the oral drug dosage form, one or more fluid inlets operably connected to the first swellable material compartment; a second swellable material compartment configured to contain at least a portion of the second swellable material; a second directional channel operably connected to the second swellable material compartment, wherein the second direction channel comprises a curved-shaped channel, wherein the second moveable arm comprises a second contact element configured to interface with the second swellable material in proximity to the second directional channel, wherein the second swellable material compartment and the second contact element of the second moveable arm substantially surround the second swellable material, and wherein the second moveable arm, or a portion thereof, is configured to rotate on the second axis by way of the second contact element sliding in the second direction channel such that the second moveable arm extends beyond, or further away from, the body of the oral drug dosage form, one or more fluid inlets operably connected to the second swellable material compartment; a drug, wherein the oral drug dosage form is configured to have a pre-administration state having a compact form and a post-administration state having an extended form providing the gastric retention, and wherein the extended form of the post-administration state of the oral drug dosage form is due to, at least in part, expansion of the swellable material in the presence of a gastrointestinal fluid.

In some embodiments, provided is an oral drug dosage form configured for a gastric retention, the oral drug dosage form comprising: a first swellable material; a first moveable arm and a second movable arm, wherein the first moveable arm and the second moveable arm are configured such that at least a portion of each moveable arm is extendible beyond, or further away from, a body of the oral drug dosage form via a force that is provided by the first swellable material, and wherein the first moveable arm and the second moveable arm extend in substantially opposite directions along an axis based on the directional channel; a second swellable material; a third moveable arm, wherein the third moveable arm is configured such that at least a portion of the third moveable arm is extendible beyond, or further away from, the body of the oral drug dosage form via a force that is provided by the second swellable material, and wherein the third moveable arm is configured to rotate on a first axis; a third swellable material; a fourth moveable arm, wherein the fourth moveable arm is configured such that at least a portion of the fourth moveable arm is extendible beyond, or further away from, the body of the oral drug dosage form via a force that is provided by the third swellable material, and wherein the fourth moveable arm is configured to rotate on a second axis; the body comprising: a first swellable material compartment configured to contain at least a portion of the first swellable material; a first directional channel and a second directional channel operably connected to the first swellable material compartment, wherein the first moveable arm comprises a first contact element configured to interface with the first swellable material in proximity to the first directional channel, wherein the second moveable arm comprises a second contact element configured to interface with the first swellable material in proximity to the second directional channel, wherein the first swellable material compartment and the first contact element of the first moveable arm and second contact element of the second moveable arm substantially surround the first swellable material, and wherein the first moveable arm, or a portion thereof, is configured to slide within the first directional channel such that the first moveable arm extends beyond, or further away from, the body of the oral drug dosage form along the axis, and wherein the second moveable arm, or a portion thereof, is configured to slide within the second directional channel such that the second moveable arm extends beyond, or further away from, the body of the oral drug dosage form along the axis; one or more fluid inlets operably connected to the first swellable material compartment; a first stop configured to engage with the first moveable arm at an extended position; a second stop configured to engage with the second moveable arm at an extended position; a second swellable material compartment configured to contain at least a portion of the second swellable material; a third directional channel operably connected to the second swellable material compartment, wherein the third direction channel comprises a curved-shaped channel, wherein the third moveable arm comprises a contact element configured to interface with the second swellable material in proximity to the third directional channel, wherein the second swellable material compartment and the contact element of the third moveable arm substantially surround the second swellable material, and wherein the third moveable arm, or a portion thereof, is configured to rotate on the first axis by way of the contact element sliding in the third direction channel such that the third moveable arm extends beyond, or further away from, the body of the oral drug dosage form, one or more fluid inlets operably connected to the third swellable material compartment; a third swellable material compartment configured to contain at least a portion of the third swellable material; a fourth directional channel operably connected to the third swellable material compartment, wherein the fourth direction channel comprises a curved-shaped channel, wherein the fourth moveable arm comprises a contact element configured to interface with the third swellable material in proximity to the fourth directional channel, wherein the third swellable material compartment and the contact element of the fourth moveable arm substantially surround the third swellable material, and wherein the fourth moveable arm, or a portion thereof, is configured to rotate on the second axis by way of the contact element sliding in the fourth direction channel such that the fourth moveable arm extends beyond, or further away from, the body of the oral drug dosage form, one or more fluid inlets operably connected to the third swellable material compartment; and a drug, wherein the oral drug dosage form is configured to have a pre-administration state having a compact form and a post-administration state having an extended form providing the gastric retention, and wherein the extended form of the post-administration state of the oral drug dosage form is due to, at least in part, expansion of the first swellable material, the second swellable material, and the third swellable material in the presence of a gastrointestinal fluid.

In some embodiments, provided is an oral drug dosage form configured for a gastric retention, the oral drug dosage form comprising: a swellable material; a first moveable arm and a second movable arm, wherein the first moveable arm and the second moveable arm are configured such that at least a portion of each moveable arm is extendible beyond, or further away from, a body of the oral drug dosage form via a force that is provided by the swellable material, and wherein the first moveable arm and the second moveable arm are configured to rotate on a shared axis in opposite directions; the body comprising: a swellable material compartment configured to contain at least a portion of the swellable material; a first directional channel and a second directional channel operably connected to the swellable material compartment, wherein the first directional channel and the second directional channel are curved-shaped and configured around the shared axis, wherein the first moveable arm comprises a first contact element configured to interface with the swellable material in proximity to the first directional channel, wherein the first contact element is rudder-shaped and configured to travel in the first directional channel such that the first moveable arm rotates beyond, or further away from, the body of the oral drug dosage form relative to the shared axis, wherein the second moveable arm comprises a second contact element configured to interface with the swellable material in proximity to the second directional channel, wherein the second contact element is rudder-shaped and configured to travel in the second directional channel such that the second moveable arm rotates beyond, or further away from, the body of the oral drug dosage form relative to the shared axis, one or more fluid inlets operably connected to the swellable material compartment; a stop, or one or more stops, configured to engage with the first moveable arm and the second moveable arm at an extended position; a drug, wherein the oral drug dosage form is configured to have a pre-administration state having a compact form and a post-administration state having an extended form providing the gastric retention, and wherein the extended form of the post-administration state of the oral drug dosage form is due to, at least in part, expansion of the swellable material in the presence of a gastrointestinal fluid. In some embodiments, the stop, or one or more stops, is configured to engage with the first contact element of the first moveable arm and the second contact element of the second moveable arm at the extended position.

In some embodiments, provided is an oral drug dosage form configured for a gastric retention, the oral drug dosage form comprising: a swellable material; a first moveable arm, a second movable arm, a third moveable arm, and a fourth moveable arm, wherein the first moveable arm, the second moveable arm, the third moveable arm, the fourth moveable arm are configured such that each arm rotates on an independent axis in a direction towards a perpendicularly situated plane via a force that is provided by the swellable material, a body comprising: a swellable material compartment configured to contain at least a portion of the swellable material; a directional channel and/or an orifice operably connected to the swellable material compartment, wherein each moveable arm comprises a contact element configured to interface with the swellable material in proximity to the directional channel and/or the orifice, and wherein the swellable material compartment, the contact elements of the moveable arms, and a cap substantially surround the swellable material; one or more fluid inlets operably connected to the swellable material compartment; a cap; a first stop, a second stop, a third stop, and a fourth stop configured to engage with the first moveable arm, second moveable arm, third moveable arm, and the fourth moveable arm, respectively, at an extended position; and a drug, wherein the oral drug dosage form is configured to have a pre-administration state having a compact form and a post-administration state having an extended form providing the gastric retention, and wherein the extended form of the post-administration state of the oral drug dosage form is due to, at least in part, expansion of the swellable material in the presence of a gastrointestinal fluid. In some embodiments, the oral drug dosage form further comprises a fifth moveable arm. In some embodiments, the oral drug dosage form further comprises a sixth moveable arm. In some embodiments, the oral drug dosage form further comprises an erodible restrainer configured to inhibit extension of the moveable arm. In some embodiments, the swellable material is configured with an associated non-swellable material covering at least a portion of the swellable material, and in such embodiments, the associated non-swellable material can contact the moveable arms.

In some embodiments, provided is an oral drug dosage form configured for a gastric retention, the oral drug dosage form comprising: a swellable material; a first moveable arm, a second movable arm, a third moveable arm, and a fourth moveable arm, wherein the first moveable arm, the second moveable arm, the third moveable arm, the fourth moveable arm are configured such that each arm rotates on an independent axis in a direction towards a perpendicularly situated plane via a force that is provided by the swellable material, a body comprising: a swellable material compartment configured to contain at least a portion of the swellable material; a directional channel and/or an orifice operably connected to the swellable material compartment, wherein each moveable arm comprises a contact element configured to interface with the swellable material in proximity to the directional channel and/or the orifice, and wherein the swellable material compartment and the contact elements of the moveable arms substantially surround the swellable material; one or more fluid inlets operably connected to the swellable material compartment; a first stop, a second stop, a third stop, and a fourth stop configured to engage with the first moveable arm, second moveable arm, third moveable arm, and the fourth moveable arm, respectively, at an extended position; and a drug, wherein the oral drug dosage form is configured to have a pre-administration state having a compact form and a post-administration state having an extended form providing the gastric retention, and wherein the extended form of the post-administration state of the oral drug dosage form is due to, at least in part, expansion of the swellable material in the presence of a gastrointestinal fluid. In some embodiments, the oral drug dosage form further comprises a fifth moveable arm. In some embodiments, the oral drug dosage form further comprises a sixth moveable arm. In some embodiments, the oral drug dosage form further comprises an erodible restrainer configured to inhibit extension of the moveable arm. In some embodiments, the swellable material is configured with an associated non-swellable material covering at least a portion of the swellable material, and in such embodiments, the associated non-swellable material can contact the moveable arms.

In some embodiments, provided is an oral drug dosage form configured for a gastric retention, the oral drug dosage form comprising: a swellable material, wherein the swellable material is wrapped, at least in part, with a semi-permeable membrane; a first moveable arm, a second movable arm, a third moveable arm, and a fourth moveable arm, wherein the first moveable arm, the second moveable arm, the third moveable arm, the fourth moveable arm are configured such that each arm rotates on an independent axis in a direction towards a perpendicularly situated plane via a force that is provided by the swellable material; and a body comprising: a first point of connection for the first arm, a second point of connection for the second arm, a third point of connection for the third arm, and a fourth point of connection for the fourth arm, wherein each point of connection is configured to provide the independent axis for rotation of the first arm, the second arm, the third arm, and the fourth arm, wherein each moveable arm comprises a contact element configured to interface with the swellable material or the semi-permeable material, and a first stop, a second stop, a third stop, and a fourth stop configured to engage with the first moveable arm, second moveable arm, third moveable arm, and the fourth moveable arm, respectively, at an extended position, wherein the oral drug dosage form is configured to have a pre-administration state having a compact form and a post-administration state having an extended form providing the gastric retention, and wherein the extended form of the post-administration state of the oral drug dosage form is due to, at least in part, expansion of the swellable material in the presence of a gastrointestinal fluid. In some embodiments, the oral drug dosage form further comprises a fifth moveable arm. In some embodiments, the oral drug dosage form further comprises a sixth moveable arm. In some embodiments, the oral drug dosage form further comprises an erodible restrainer configured to inhibit extension of the moveable arm. In some embodiments, the swellable material is configured with an associated non-swellable material covering at least a portion of the swellable material, and in such embodiments, the associated non-swellable material can contact the moveable arms.

### I. Drug Dosage Forms Configured for Other Cavities

The concept of pharmaceutical dosage forms or medical devices of the present invention may not only be applied to oral administration, but also to other parts of the human body, such as the large intestine, small intestine, rectum, cecum, colon, vagina and other parts, the pharmaceutical dosage form or medical devices described in the present invention is also applicable. One of ordinary skill in the art will readily appreciate that these other cavities may have anatomical features that guide taught characteristics of the drug dosage forms described herein. For example, the anatomical size of the rectum can guide the size of the pre-administration drug dosage form for such use, which, in certain embodiments, may differ from that of an oral drug dosage form. That being said, the mechanisms taught herein for retention in an individual are still applicable.

### III. Commercial Batches

In some aspects, provided herein is a commercial batch of an oral drug dosage form described herein. In some embodiments, the commercial batch comprises at least about any of 100, 150, 200, 250, 500, 750, 1,000, 2,500, 5,000, 7,500, 10,000, 20,000, 30,000, 40,000, 50,000, 60,000, 70,000, 80,000, 90,000 or 100,000 of an oral drug dosage forms described herein. In some embodiments, each of the oral drug dosage forms of the commercial batch is produced using the same technique, such as involving via three-dimensional (3D) printing, injection molding, ultrasonic welding, or any combination thereof, of one or more components of the oral drug dosage form. In some embodiments, the commercial batch has a standard deviation of about 0.1 or less, such as 0.05 or less, for one or more of the following: an amount of a drug in the oral drug dosage form; weight of the oral drug dosage form; dimensions of the oral drug dosage form (such as in the pre-administration state and/or post-administration state); and time of stomach retention of the oral drug dosage form. In some embodiments, the dimension of the oral drug dosage form is a largest crossing dimension of a pre-administration state of an oral drug dosage form. In some embodiments, the dimension of the oral drug dosage form is a crossing dimension perpendicular to the largest crossing dimension of the oral drug dosage form in a pre-administration state. In some embodiments, the dimension of the oral drug dosage form is a largest crossing dimension of the oral drug dosage form after swelling of the swellable material (such as in a post-administration state in the stomach wherein the swellable material has swelled to a substantially complete state). In some embodiments, the dimension of the oral drug dosage form is a crossing dimension perpendicular to the largest crossing dimension of the oral drug dosage form after swelling of the swellable material (such as in a post-administration state in the stomach wherein the swellable material has swelled to a substantially complete state).

### IV. Methods of Making

In some aspects, provided herein are methods of making an oral drug dosage form described herein. In some embodiments, the method of making comprises a three-dimensional (3D) printing technique to form at least one of the components, or a portion thereof, of the drug dosage forms described herein. In some embodiments, the method of making comprises use of 3D printing, injection molding, ultrasonic welding, or any combination thereof. In some embodiments, components of an oral drug dosage form taught herein are produced separately and later assembled, by machine and/or hand.

As used herein, “printing,” “three-dimensional printing,” “3D printing,” “additive manufacturing,” or equivalents thereof, refers to a process that produces three-dimensional objects, such as delayed sustained-release oral drug dosage forms, layer-by-layer using digital designs. The basic process of three-dimensional printing has been described in U.S. Pat. Nos. 5,204,055; 5,260,009; 5,340,656; 5,387,380; 5,503,785; and 5,633,021. Additional U.S. patents and patent applications that related to three-dimensional printing include: U.S. Pat. Nos. 5,490,962; 5,518,690; 5,869,170; 6,530,958; 6,280,771; 6,514,518; 6,471,992; 8,828,411; U.S. Publication Nos. 2002/0015728; 2002/0106412; 2003/0143268; 2003/0198677; 2004/0005360. The contents of the above U.S. patents and patent applications are hereby incorporated herein by reference in their entirety. In some embodiments, an additive manufacturing technique is used to produce the oral drug dosage forms described herein, or components thereof. In some embodiments, a layer-by-layer technique is used to produce the oral drug dosage forms described herein, or components thereof. For example, in some embodiments, the layer-by-layer technique comprises dispensing one or more materials of an entire first layer of an oral drug dosage form, or component thereof, and then proceeding to dispense one or more materials of an entire second layer of the oral drug dosage form, or component thereof. In some embodiments, the layer, such as the first layer or second layer, is a cross-section of an oral drug dosage form, or component thereof. Because 3D printing may handle a range of pharmaceutical materials and control both composition and architecture locally, 3D printing is well suited to the fabrication of drug dosage forms with complex geometry and compositions in accordance with the present invention.

In some embodiments, layer, when used in reference to a components of the drug dosage form, e.g., a swellable material, refers to the configuration of a component of the oral drug dosage form and may comprise a plurality of printed layers of the same material. In some embodiments, the layer has a pre-determined fill density, such a three-dimensional printed fill density. In some embodiments, the layer comprises a plurality of printed layers between about 5 printed layers to about 2500 printed layers, such as between any of about 10 printed layers to about 2500 printed layers, about 25 printed layers to about 100 printed layers, about 50 printed layers to about 200 printed layers, about 100 printed layers to about 200 printed layers, about 150 printed layers to about 250 printed layers, about 200 printed layers to about 250 printed layers, about 500 printed layers to about 1000 printed layers, or about 2000 printed layers to about 2400 printed layers. In some embodiments, the thickness of a printed layer is no more than about 5 mm, such as no more than about any of 4 mm, 3 mm, 2 mm, 1 mm, 0.9 mm, 0.8 mm, 0.7 mm, 0.6 mm, 0.5 mm, 0.4 mm, 0.3 mm, 0.2 mm, 0.1 mm, 0.09 mm, 0.08 mm, 0.07 mm, 0.06 mm, 0.05 mm, 0.04 mm, 0.03 mm, 0.02 mm, or 0.01 mm. In some embodiments, the thickness of a printed layer is about any of 5 mm, 4 mm, 3 mm, 2 mm, 1 mm, 0.9 mm, 0.8 mm, 0.7 mm, 0.6 mm, 0.5 mm, 0.4 mm, 0.3 mm, 0.2 mm, 0.1 mm, 0.09 mm, 0.08 mm, 0.07 mm, 0.06 mm, 0.05 mm, 0.04 mm, 0.03 mm, 0.02 mm, or 0.01 mm.

Different 3D printing methods have been developed for manufacturing in terms of raw materials, equipment, and solidification. These 3D printing methods include binder deposition (see Gibson et al., Additive Manufacturing Technologies: 3D Printing, Rapid Prototyping, and Direct Digital Manufacturing., 2 ed. Springer, New York, 2015; Katstra et al., Oral dosage forms fabricated by three dimensional printing, J Control Release, 66, 2000; Katstra et al., Fabrication of complex oral delivery forms by three dimensional printing, Dissertation in Materials Science and Engineering, Massachusetts Institute of Technology, 2001; Lipson et al., Fabricated: The New World of 3D printing, John Wiley &Sons, Inc., 2013; Jonathan, Karim 3D printing in pharmaceutics: a new tool for designing customized drug delivery systems, Int J Pharm, 499, 2016), material jetting (see Jonathan, Karim, 3D printing in pharmaceutics: a new tool for designing customized drug delivery systems, Int J Pharm, 499, 2016), extrusion (see Gibson et al., Additive Manufacturing Technologies: 3D Printing, Rapid Prototyping, and Direct Digital Manufacturing. 2 ed. Springer, New York, 2015), and photopolymerization (see Melchels et al., A review on stereolithography and its application in biomedical engineering. Biomaterials, 31, 2010).

In some embodiments, the oral drug dosage forms described herein are 3D printed using an extrusion method. In some embodiments, the method of 3D printing comprises using a double screw extrusion method. In an extrusion process, material is extruded from robotically-actuated printing heads through printing nozzles. Unlike binder deposition, which requires a powder bed, extrusion methods can print on any substrate. A variety of materials can be extruded for three-dimensional printing, including thermoplastic materials disclosed herein, pastes and colloidal suspensions, silicones, and other semisolids. One extrusion printing method is melt extrusion deposition (MED), which used extruded material from a printing head to print layers of material to form the components of the oral drug dosage form, or component thereof. Another common type of extrusion printing is fused deposition modeling, which uses solid polymeric filaments for printing. In fused deposition modeling, a gear system drives the filament into a heated nozzle assembly for extrusion (see Gibson et al., Additive Manufacturing Technologies: 3D Printing, Rapid Prototyping, and Direct Digital Manufacturing, 2 ed. Springer, New York, 2015).

In some embodiments, the 3D printing is carried out by melt extrusion deposition (MED). In some embodiments, the melt extrusion deposition technique comprises preparing a material to be dispensed, such as preparing a powder in a hot melt extruder, and then feeding the material into a MED printing head. The MED printing head then dispenses the material to form the oral drug dosage form, or component thereof, in an additive manner (layer-by-layer deposition). In some embodiments, each material of the oral drug dosage form, or component thereof, is dispensed from a different MED printing head. In some embodiments, the MED printing head dispenses the material according to instructions complied in one or more gcode files. Exemplary MED techniques are disclosed in, e.g., WO2019/137333, WO2018137686, and U.S. Pat. No. 10,201,503, each of which is incorporated herein by reference in its entirety.

In some embodiments, the 3D printing is carried out by fused deposition modeling (FDM). In some embodiments, the three-dimensional printing is carried out by melt extrusion deposition or hot melt extrusion coupled with a 3D printing technique, such as FDM. In some embodiments, the 3D printing is carried out by non-filament FDM. In some embodiments, the 3D printing is carried out by inkjet printing. In some embodiments, the 3D printing is carried out by selective laser sintering (SLS). In some embodiments, the 3D printing is carried out by stereolithography (SLA or SL). In some embodiments, the 3D printing is carried out by PolyJet, Multi-Jet Printing System (MJP), Perfactory, Solid Object Ultraviolet-Laser Printer, Bioplotter, 3D Bioprinting, Rapid Freeze Prototyping, Benchtop System, Selective Deposition Lamination (SDL), Laminated Objet Manufacutring (LOM), Ultrasonic Consolidation, ColorJet Printing (CJP), EOSINT Systems, Laser Engineered Net Shaping (LENS) and Aerosol Jet System, Electron Beam Melting (EBM), Laser CUSING®, Selective Laser Melting (SLM), Phenix PX™ Series, Microsintering, Digital Part Materialization (DPM), or VX System.

In some embodiments, the 3D printing methods described herein comprise a continuous feed method. In some embodiments, the 3D printing methods described herein comprise a batch feed method.

In some embodiments, the methods for producing the drug dosage forms described herein comprise a 3D printing technique, such as 3D printing in combination with another method, e.g., a combination of injection molding and 3D printing. In some embodiments, the method for producing further comprises an injection molding technique. In some embodiments, the method for producing further comprises a ultrasonic welding method. In some embodiments, 3D printing techniques, injection molding techniques, and ultrasonic welding methods can be used separately or in any combination.

The method instructions for 3D printing a drug dosage form disclosed herein may be generated a variety of ways, including direct coding, derivation from a solid CAD model, or other means specific to the 3D printing machine's computer interface and application software. These instructions may include information on the number and spatial placement of droplets, and on general 3D print parameters such as the drop spacing in each linear dimension (X, Y, Z), and volume or mass of fluid per droplet. For a given set of materials, these parameters may be adjusted in order to refine the quality of structure created. The overall resolution of the structure created is a function of the powder particle size, the fluid droplet size, the print parameters, and the material properties.

In some embodiments, one or more components of the oral drug dosage form are created separately, such as printed separately, and later assembled to form the oral drug dosage form. In some embodiments, all components of the oral drug dosage form are created in a single method, such as printed in a single method, without requiring later assembly.

The oral drug dosage forms, and components thereof, described in the present application can be printed on a commercial scale. For example, in some embodiments, the methods disclosed herein may be used to 3D print 1,000 to 100,000 units of an oral drug dosage form per hour, including printing the components there of for later assembly.

In some embodiments, the materials used to print the oral drug dosage forms, or components thereof, are each dispensed by a different printing head. The 3D printing methods described herein encompass printing the materials in any order that will allow for production of the oral drug dosage form, or components thereof.

In some embodiments, the method for 3D printing comprises designing the oral drug dosage form, or component thereof, in whole or in part, on a computer system. In some embodiments, the method comprises inputting parameters of the desired gastric retention, drug release profile, and/or the oral drug dosage form and/or the component(s) into the computer system. In some embodiments, the method comprises providing one or more parameters to be printed, e.g., layer surface area, thickness, drug mass fraction, erosion rate. In some embodiments, the method comprises providing the desired drug release profile. In some embodiments, the methods comprise creating a virtual image of the item to be printed. In some embodiments, the method comprises creating a computer model that contains the pre-determined parameters. In some embodiments, the method comprises feeding the pre-determined parameters to a 3D printer and printing the item according to such pre-determined parameters. In some embodiments, the method comprises creating a 3D drawing of the item to be printed based on the pre-determined parameters, wherein the 3D drawing is created on a computer system. In some embodiments, the method comprises converting, such as slicing, a 3D drawing into 3D printing code, e.g., G code. In some embodiments, the method comprises using the computer system to execute 3D printing code, thereby printing according to the methods described herein.

### V. Methods of Delivering a Drug to an Individual

In some aspects, provided is a method of delivering an oral drug dosage form to an individual such that the oral drug dosage form is retained in the stomach of the individual for an extended time, the method comprising orally administering to the individual any oral drug dosage form described herein. In some aspects, provided is a method of delivering a drug to the stomach and/or upper gastrointestinal tract of an individual, the method comprising orally administering to the individual any oral drug dosage form described herein, wherein the oral drug dosage form comprises the drug. In some embodiments, the drug is release from the oral drug dosage form to the stomach and/or upper gastrointestinal tract over an extended period of time (including a sustained-release profile, a delayed-release profile, a pulsed-release profile, or any combination thereof). In some embodiments, the oral drug dosage form is configured to be retained in the stomach of an individual for an extended period of time (e.g., at least about 8 hours to about 3 months). In some embodiments, the drug or drugs in an oral drug dosage form can be release from an oral drug dosage form at a pre-determined time(s) after administration of the oral drug dosage form to the individual. For example, in some embodiments, the drug, or at least a portion thereof, is released in the stomach of an individual as, or following, the oral drug dosage form reaches a post-administration state designed for gastric retention. In some embodiments, the drug, or at least a portion thereof, is released in the stomach of an individual at least about any of 2 hours, 4 hours, 6 hours, 8 hours, 10 hours, 12 hours, 14 hours, 16 hours, 18 hours, 20 hours, 22 hours, 24 hours, 26 hours, 28 hours, 30 hours, 32 hours, 34 hours, or 36 hours after administering the drug dosage form to the individual.

Those skilled in the art will recognize that several embodiments are possible within the scope and spirit of the disclosure of this application. The disclosure is illustrated further by the examples below, which are not to be construed as limiting the disclosure in scope or spirit to the specific procedures described therein.

### EXAMPLES

### Example 1

This example demonstrates the design and testing of an oral drug dosage form described herein which is configured for stomach retention.

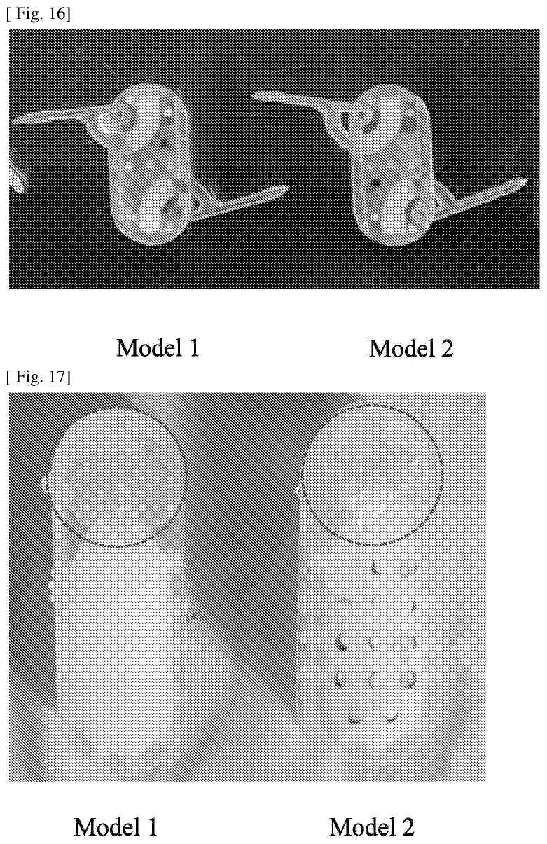

Two demonstrative models based on the design configuration shown in FIGS. 5A-5F were 3D printed with material formulation of the swellable material (LHB1:NaCl=9:1). For both models, the body and the movable arms were printed with photosensitive resin. After printing, the two demonstrative models were immersed in water such that the swellable material could swell and force the moveable arms to rotate and extend beyond, or further away from, the body of the oral drug dosage form. At certain time points, the angles between the two movable arms were measured to evaluate the extent of dimension expansions that could be achieved by the drug dosage form upon swelling. The tested material formulations of the swellable component and their corresponding results are shown in Table 1 and FIG. 16.

TABLE 1
Details of tested models.

| Model | Swelllable | |
|---|---|---|
| No. | Material | Results |
| 1 | LHB1:NaCl = 9:1 | After 4 minutes, the volume of the swellable |
| | | material expanded to about 3.5 times of its |
| | | volume prior to swelling, and the angles |
| 2 | | between two spread arms were relatively |
| | | large. |

### Example 2

This example demonstrates the design and testing of drug dosage forms described herein which are configured for stomach retention.

Two demonstrative models based on the design configuration shown in FIGS. 7A-7I were 3D printed. The two demonstrative models were printed with different designs of the fluid inlets on the body that allowed fluid into the swellable material compartment. One model of the body had fluid inlets only on the side wall of the swellable compartment, while the other model of the body had fluid inlets on the top, bottom, and side walls of the swellable material compartment. The direction channel on the body part was capped on two sides by two caps in place of the movable arms. All the fluid inlets were Φ1 mm. The third channel which connects the swellable compartment and the first directional channel in the body had a cross section of 3.5 mm×4 mm. The swellable material used for this test was PAANa (30-60 mesh). The rest of the aspects of the body and the two caps were printed with photosensitive resin.

After printing, the models were immersed in a fluid with a pH of 1.0 such that the side(s) of the swellable compartment and fluid inlets in the body were immersed while the side of the first directional channel with caps was left above the fluid. The extent to which the swellable material extended beyond the swellable material compartment and filled the first directional channel within a certain amount of time was tested and recorded.

The designs of the fluid inlets in the two demonstrative models are shown in Table 2.

TABLE 2
Details of fluid inlets of the models.

| | | Numbers of inlets on | |
|---|---|---|---|
| Design of | Numbers of inlets on | the top and bottom | Size of |
| fluid inlets | the side wall | walls | the inlets |
| Model 1 | 11 per row × 2 rows | N/A | Φ1 mm |
| Model 2 | 11 per row × 2 rows | 14 per row × 2 rows | Φ1 mm |

Both model 1 and model 2 had the swellable material fully fill the first directional channel within 2 minutes (FIG. 17).

### Example 3

This example demonstrates the design and testing of oral drug dosage forms described herein which are configured for stomach retention.

Ten replicate demonstrative models based on the design configuration shown in FIGS. 8A-8I were 3D printed. The methods of preparation, certain functions, and materials of component of the oral drug dosage form are summarized in Table 3.

TABLE 3
Details of certain components of the oral drug dosage form.

| Component | Method of preparation | Features and functions |
|---|---|---|
| Erodible | In-house printing | The sleeve-shaped restrainer can bundle the arms and inhibit extension |
| restrainer | | of the moveable arms before the swelling of swellable material. The |
| | | crodible restrainer can dissolve in water. |
| Movable arms | In-house printing | The movable arms can have “C-shape hooks on one end and be |
| | | snapped-locked onto the body. This configuration can achieve reliable |
| | | conncetion and flexible rotation. The arms can be printed with imaging |
| | | agent(s) and/or be attached with strips containing imaging agent(s) and |
| | | used for imaging. |
| Swellable | In-house printing | The swellable material compartment can be used to store the swellable |
| material | | agent(s), propel the movable arms and sustain the fully extended state |
| compartment | | of the movable arms. The swellable material compartment can be |
| | | printed with imaging agent(s) and used for imaging. |
| Cap of body | SLA printing | The cap can be stabilized on top of the bottom frame by photocurable |
| | | resin and prevents the swellable material compartment from leaving. |
| | | the bottom frame upon swelling. |
| Bottom frame of | SLA printing | The bottom frame can have seven Φ1 mm fluid inlots configured to |
| body. | | permit passage of fluid. |

For each demonstrative model, the bottom frame and cap were printed with photosensitive resin. The swellable material compartment was printed with EC:ATBC:BaSO₄=65:15:20. The erodible restrainer was printed with VA64:TEC=9:1. The swellable material was super absorbent polymer (30-60 mesh). The movable arms were printed with EC:ATBC:BaSO₄=60:20:20. Five of the demonstrative models were put into water with the bottom frame facing down and arm side facing up (extending-upwards configuration), while the other five of demonstrative models were put into water with the bottom frame facing up and arm side facing down (extending-downwards configuration). The extents to which the arms extend out upon swelling under different conditions are shown in FIG. 18. As shown in FIG. 18, demonstrative models of the oral drug dosage form were able to form to 30 mm×30 mm post-administration state within 30 minutes.

### Example 4

This example demonstrates the design and testing of oral drug dosage forms described herein which are configured for stomach retention.

Ten replicate demonstrative models based on the design configuration shown in FIGS. 9A-9I were manufactured. The methods of preparation, certain functions, and materials of component of the oral drug dosage form are summarized in Table 4.

TABLE 4
Details of certain components of the oral drug dosage form.

| Component | Method of preparation | Features and functions |
|---|---|---|
| Erodible | In-house printing | The sleeve-shaped restrainer can bundle the arms and inhibit extension |
| restrainer | | of the moveable arms before the swelling of swellable material. |
| Arm | SLA printing | The movable arms can have joint pins on one end and be snapped- |
| | | locked onto the body. This configuration can achieve reliable |
| | | connection and flexible rotation. The arms can be printed with imaging |
| | | agent(s) and/or be attached with strips containing imaging agent(s) and |
| | | used for imaging. |
| Cap of the body | SLA printing | The cap can be stabilized on top of the bottom frame by photocurable |
| | | resin and prevents the swellable material compartment from leaving |
| | | the bottom frame upon swelling. |
| Bottom frame of | SLA printing | The bottom frame can have five Φ1 mm fluid inlets configured to |
| the body | | permit passage of fluid. |
| Swellable | In-house printing | The swellable material compartment can be used to store the swellable |
| material | | agent(s), propel the movable arms and sustain the fully extended state |
| compartment | | of the movable arms. The swellable material compartment can be |
| | | printed with imaging agent(s) and used for imaging. |

For each demonstrative model, the bottom frame, cap, and movable arms were printed with photosensitive resin. The swellable material compartment was printed with EC:ATBC:BaSO₄=65:15:20. The erodible restrainer was printed with VA64:TEC=9:1. The swellable component was printed with super absorbent polymer (30-60 mesh). These ten demonstrative models were immersed in water and the arm measurements were taken over time. The extent to which the arms spread out upon swelling is shown in Table 5.

TABLE 5
Arm extension measurements for each demonstrative model.

| | | Dimensions of |
|---|---|---|
| Model | Time | the drug dosage |
| No. | (min) | form/mm × mm |
| 1 | 4 | 39.37 × 39.68 |
| 2 | 9 | 39.47 × 39.98 |
| 3 | 14 | 39.64 × 39.72 |
| 4 | 30 | 28.14 × 7.40 |
| 5 | 60 | 30.48 × 29.00 |
| 6 | 15 | 36.25 × 33.01 |
| 7 | 6 | 37.17 × 37.30 |
| 8 | 7 | 39.14 × 39.03 |
| 9 | 14 | 36.54 × 36.47 |
| 10 | 20 | 36.23 × 34.53 |

The expansion of the dimensions of the oral drug dosage forms upon swelling was also examined in vivo. To enable in vivo imaging, BaSO₄ was included in the formulation of the swellable material compartment. In addition, strips with imaging agent were attached onto the surface of the movable arms for imagine of the arm position.

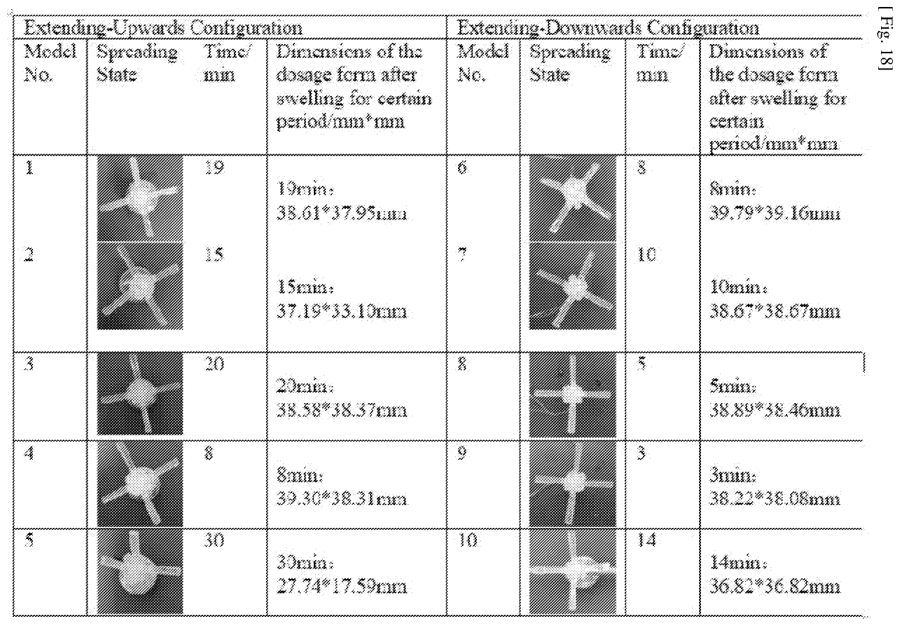

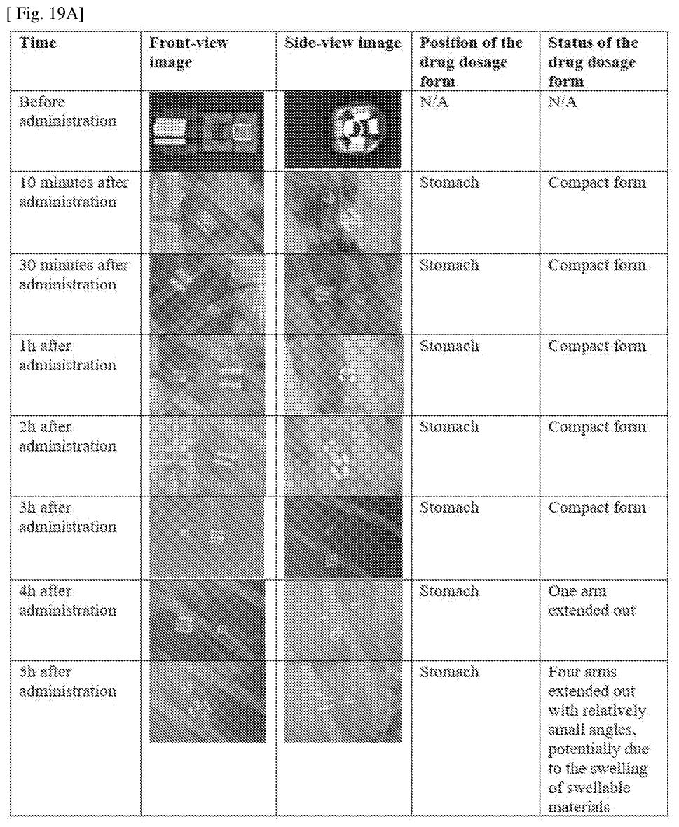

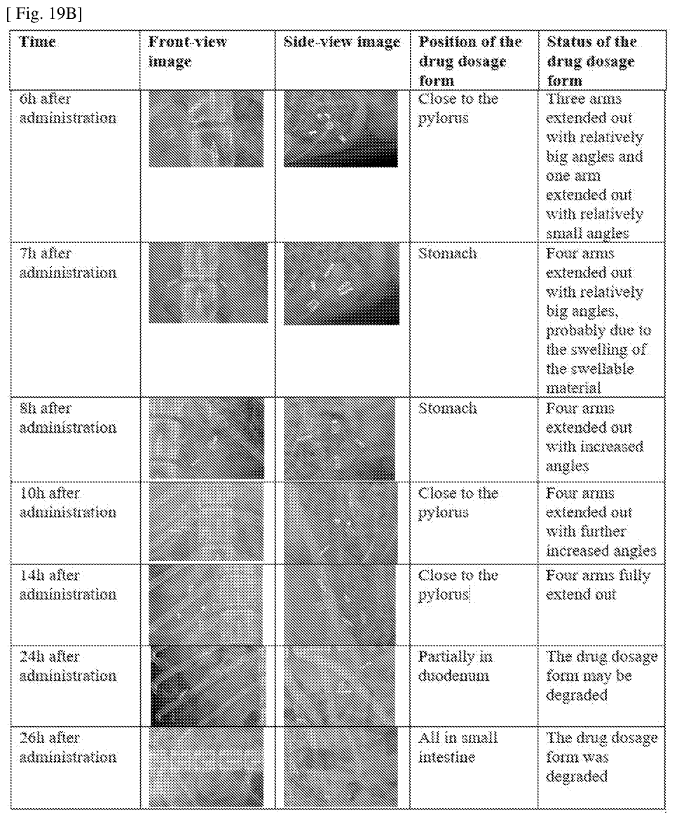

One male Beagle dog was used for this study. The dog was fasted for 14 hours while allowed to drink water prior to the administration of the oral drug dosage form. Dog food was provided 4 hours after the administration of the oral drug dosage form and water was supplied throughout the experiment. The drug dosage form was swallowed as a whole with 20 mL water without being cracked, chewed, or grounded. To evaluate the retention time and status of the oral drug dosage form, X-ray images of the drug dosage form were collected before the administration and 10 minutes, 30 minutes, 1 h, 3 h, 4 h, 5 h, 6 h, 7 h, 8 h, 10 h, 14 h, 24 h, and 26 h after administration. For each time point, both front-view and side-view images were taken. The collected X-ray images, the position of the drug dosage form, and the extent to which the moveable arms extend out at each collection time point are provided in FIGS. 19A and 19B. As shown in FIGS. 19A and 19B, the oral drug dosage form was retained in the stomach for an extended period of time with the moveable arms in an extended position, and the oral drug dosage form was able to stay in stomach for at least about 14 hours.

### Example 5

This example demonstrates the design and testing of oral drug dosage forms described herein which are configured for stomach retention.

The demonstrative models based on the design configuration shown in FIGS. 14A-14B were manufactured.

For the demonstrative model, the bottom frame, cap, and movable arms were injection molded with PLA and ABS. The swellable component was tableted with PEO 700W:CCNa:BaSO4=56:24:20. The erodible restrainer was printed with VA64:TEC=85:15.

The expansion of the dimensions of the oral drug dosage forms upon swelling was examined in vivo. To enable in vivo imaging, BaSO₄ was included in the formulation of the swellable material compartment. In addition, strips with imaging agent were attached onto the surface of the movable arms for imagine of the arm position.

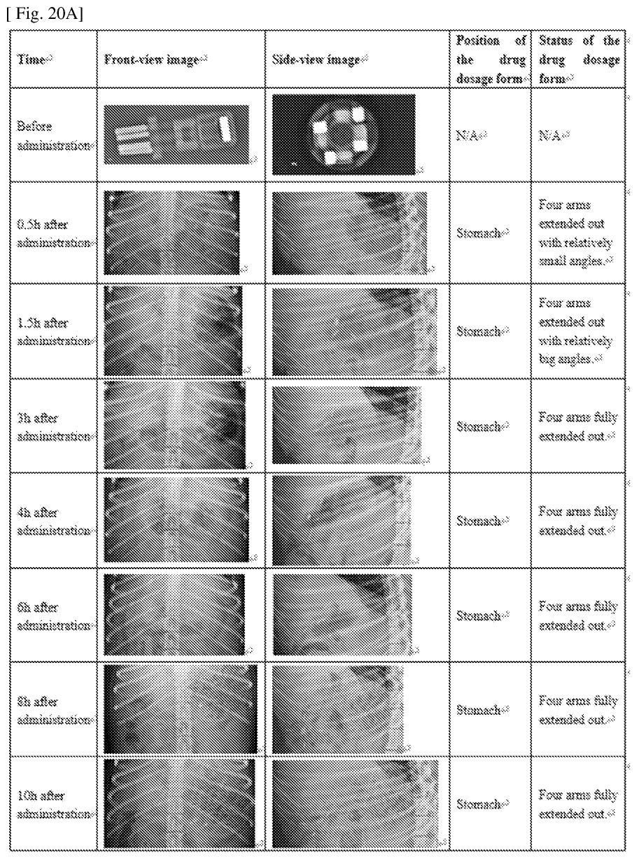

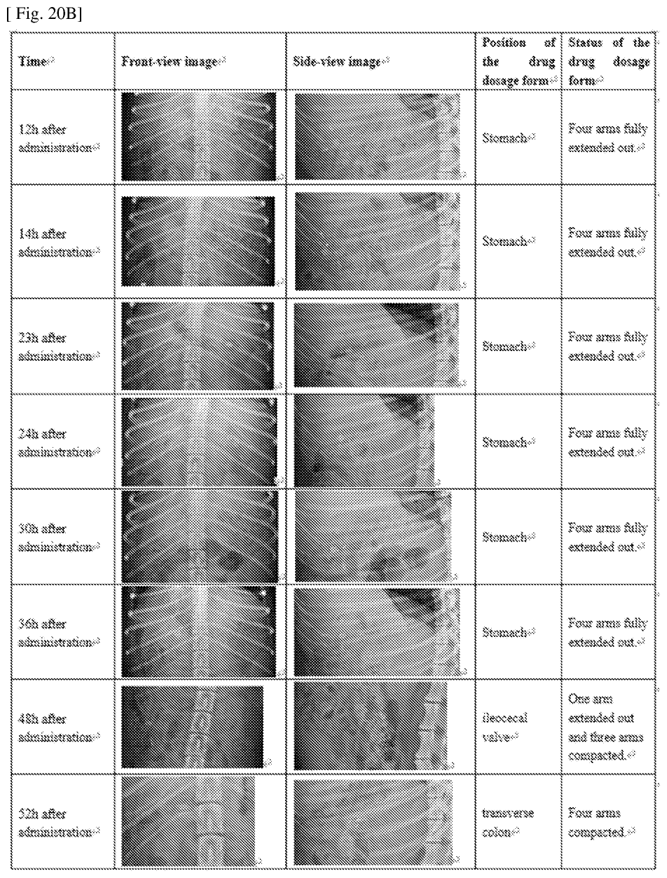

One male Beagle dog was used for this study. The dog was fasted for 14 hours while allowed to drink water prior to the administration of the oral drug dosage form. Dog food was provided 4 hours after the administration of the oral drug dosage form and water was supplied throughout the experiment. The drug dosage form was swallowed as a whole with 20 mL water without being cracked, chewed, or grounded. To evaluate the retention time and status of the oral drug dosage form, X-ray images of the drug dosage form were collected before the administration and 30 minutes, 1.5 h, 3 h, 4 h, 6 h, 8 h, 10 h, 12 h, 14 h, 23 h, 24 h, 30 h, 36 h, 48 h and 52 h after administration. For each time point, both front-view and side-view images were taken. The collected X-ray images, the position of the drug dosage form, and the extent to which the moveable arms extend out at each collection time point are provided in FIGS. 20A and 20B. As shown in FIGS. 20A and 20B, the oral drug dosage form was retained in the stomach for an extended period of time with the moveable arms in an extended position, and the oral drug dosage form was able to stay in stomach for at least about 36 hours.

### Example 6

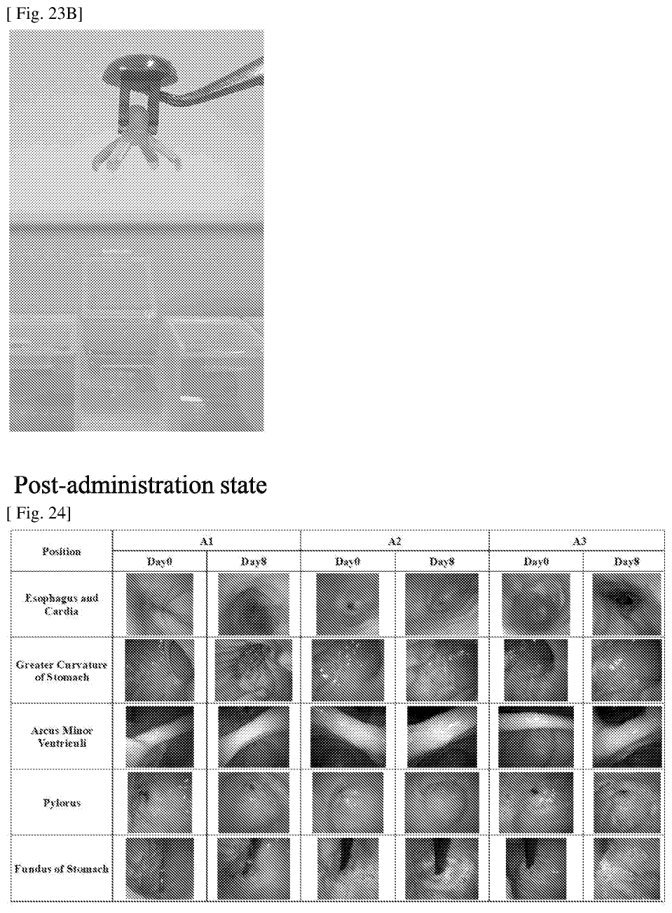

The drug dosage form model was fixed in the fixture and the bottom was then immersed in 5 mL of purified water medium to observe the deployment of the unfolded arm as shown in FIG. 22A. It was found that the unfolded arm was unfolded for 120° in 5 minutes, as shown in FIG. 23B. The above experiments show that the drug dosage form retention in vivo can be developed in less fluid, with potential applications for colonic retention or vaginal retention.

### Example 7

This example demonstrates the design and testing of oral drug dosage forms described herein which are configured for stomach retention.

The demonstrative models based on the design configuration shown in FIGS. 22A-22B were manufactured.

For the demonstrative model, the bottom frame, cap, and movable arms were made with HPMC, and the swellable component was tableted with PEO.

This study was conducted to evaluate the in-vivo safety after oral administration of placebo candidate formulation once daily for 7 consecutive days by gastroscopy examinations, fecal occult blood (OB) tests, and collected residues from the feces.

Placebo candidate formulation was orally administrated once daily for 7 consecutive days by 3 beagle dogs under fed conditions. Gastroscopy examinations were carried out on day 0 (pre dose) and day 8 (after the last dosing), and the OB test were conducted daily to evaluate the in-vivo safety when placebo candidate formulations were orally administrated by beagle dogs.

Gastroscopy examination for 3 beagle dogs which orally administrated placebo candidate for 7 consecutive days showed that no change was observed at day 8 when compared to day 0, which demonstrated that no directly observable damage was occurred during dosing. The gastroscopy examination (Day 0 and Day 8) provided in FIG. 24.

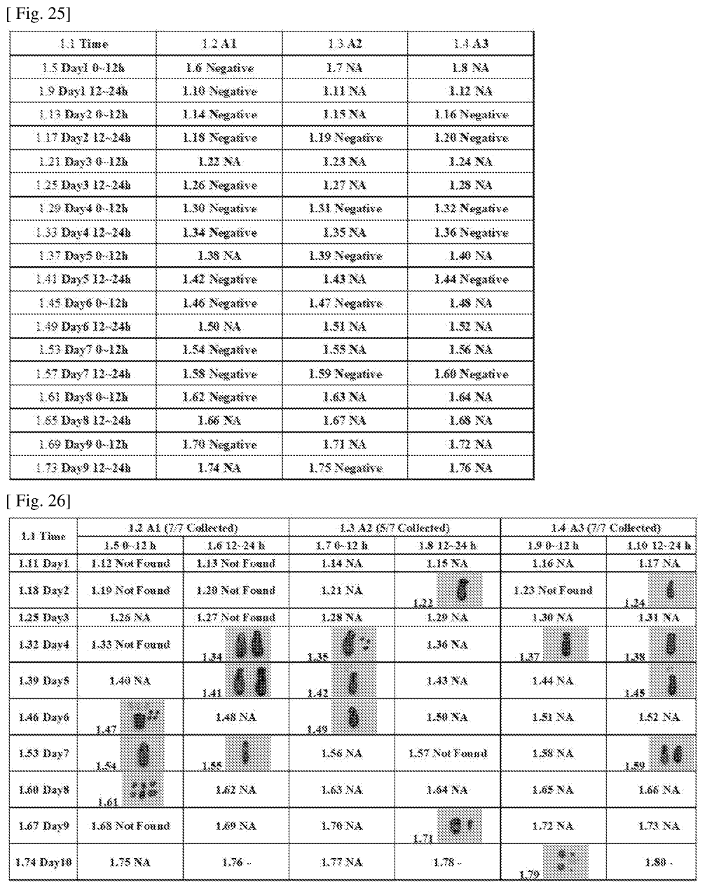

The Occult Blood test results of the collected feces during 7 days consecutive dosing were all negative, indicating that placebo candidate formulation didn't cause any damage to the GI tract of beagle dogs during GI transition. Results of Fecal Occult Blood Tests shown in FIG. 25.

The residues of placebo candidate formulation were collected and all the residues were soft. The pictures of Collected Residues from Beagle Dogs' Feces shown in FIG. 26.

The beagle dogs were eating and acting normally after oral administration of place candidate formulation for 7 consecutive days. No damage in the GI tract of beagle dogs were observed during dosing.

1. An oral drug dosage form configured for gastric retention, the oral drug dosage form comprising:
a swellable material;
a moveable arm configured such that at least a portion of the moveable arm is extendible beyond, or rotate around the body a body of the oral drug dosage form via a force that is provided by the swellable material; and
the body comprising:
a swellable material compartment configured to contain at least a portion of the swellable material;
wherein the swellable material compartment operably comprises one or more fluid inlets;
a directional channel and an orifice operably connected to the swellable material compartment,
wherein the moveable arm comprises a contact element configured to interface with the swellable material, or a feature associated with the swellable material, in proximity to the directional channel and the orifice; and
a drug,
wherein the oral drug dosage form is configured to have a pre-administration state having a compact form and a post-administration state having an extended form providing gastric retention, and wherein the extended form of the post-administration state of the oral drug dosage form is due to, at least in part, expansion of the swellable material in the presence of a gastrointestinal fluid.

2. The oral drug dosage form of claim 1, wherein the feature associated with the swellable material comprises a plunger or a piston.

3. The oral drug dosage form of claim 1, wherein the movable arm or body comprises a pivot, and the movable arm is connected to the body by pivoting.

4. The oral drug dosage form of claim 1, wherein the swellable material compartment comprises a cap and a base, and the pivot of the movable arm is connected to the cap.

5. The oral drug dosage form of any one of claims 1-4, wherein the post-administration state of the oral drug dosage form occurs within 2 hour or less following administration of the oral drug dosage form to the individual.

6. The oral drug dosage form of any one of claims 1-4, wherein the gastric residence of the oral drug dosage form is about 6 hours to about 3 months.

7. The oral drug dosage form of any one of claims 1-4, wherein the swellable material expands at least about 1.2× in volume following exposure to a gastrointestinal fluid.

8. The oral drug dosage form of any one of claims 1-5, wherein the swellable material has a volume expansion rate of at least about 1.2× in 30 minutes.

9. The oral drug dosage form of any one of claims 1-5, wherein the swellable material, upon swelling, at least in part conforms to the shape of the direction channel and the orifice, or a portion thereof.

10. The oral drug dosage form of any one of claims 1-5, wherein the swellable material, upon swelling, adopts a pre-determined shape and/or size.

11. The oral drug dosage form of any one of claims 1-5, wherein the swellable material in the swellable material compartment is of an amount of at least about 5 mg.

12. The oral drug dosage form of any one of claims 1-5, wherein the swellable material comprises sodium alginate (SA), hydroxypropyl cellulose (HPC), hydroxyethyl cellulose (HEC), hydroxypropyl methyl cellulose (HPMC), polyethylene oxide (PEO), polyvinyl alcohol (PVA), microcrystalline cellulose (MCC), croscarmellose sodium (CCNa), carboxymethylcellulose sodium (CMC-Na), polyvinylpolypyrrolidone (PVPP), carboxymethyl starch sodium (CMS-Na), polyethylene glycol (PEG), or a mixture thereof.

13. The oral drug dosage form of any one of claims 1-5, wherein the swellable material comprises gas-producing substance.

14. The oral drug dosage form of claim 13, wherein the swellable material further comprises a salt or a mixture of salts, wherein the salt is selected from the group consisting of a sodium salt, a magnesium salt, and a kali salt.

15. The drug dosage form of any one of claims 1-5, wherein the swellable material has a swelling volume of about 10 mm³ to about 50 mm³.

16. The drug dosage form of claim 2, where in the plunger or piston pushes the arm to rotate around the body into an unfolded form.

17. The oral drug dosage form of any one of claims 1-5, wherein the body comprises two or more pieces configured to form the body.

18. The oral drug dosage form of any one of claims 1-5, wherein the body comprises two or more materials.

19. The oral drug dosage form of any one of claims 1-5, wherein the body of the oral drug dosage form is a monolithic structure.

20. The oral drug dosage form of any one of claims 1-5, wherein the largest crossing dimension of the oral drug dosage form in a pre-administration state is 24 mm or less.

21. The oral drug dosage form of any one of claims 1-5, wherein at least two perpendicular dimension of the oral drug dosage form in a post-administration state are 20 mm or more.

22. The oral drug dosage form of any one of claims 1-5, wherein the swellable material compartment is configured to substantially contain the swellable material in a pre-administration state.

23. The oral drug dosage form of any one of claims 1-5, wherein the swellable material compartment and the contact element of the moveable arm substantially surround the swellable material.

24. The oral drug dosage form of any one of claims 1-5, wherein the directional channel is configured to direct movement of the moveable arm via the swellable material.

25. The oral drug dosage form of any one of claims 1-5, wherein the directional channel comprises a curved-shaped channel, straight-shaped channel, a circular-shaped channel, oval-shaped channel, capsule-shaped channel, a square or rectangular-shaped channel.

26. The drug dosage form of any one of claims 1-4, wherein the drug dosage form can be taken orally, or located in the stomach, small intestine, large intestine, rectum, colon, vagina.

27. The oral drug dosage form of claim 4, wherein the body or base is semi-permeable membrane.

28. The oral drug dosage form of claim 27, wherein the at least one of the one or more fluid inlets is the semi-permeable material filling a pore formed by the body.

29. The oral drug dosage form of any one of claims 1-5, wherein at least one of the one or more fluid inlets is a pore.

30. The oral drug dosage form of any one of claims 1-5, wherein the body further forms a stop configured to engage with the moveable arm at an extended position.

31. The oral drug dosage form of claim 30, wherein the stop comprises a locking element configured to maintain the moveable arm at one or more extended positions.

32. The oral drug dosage form of any one of claims 1-5, wherein the body has a wall thickness of at least about 0.4 mm.

33. The oral drug dosage form of any one of claims 1-5, wherein at least two of the moveable arms have a different movement mechanism or the same movement mechanism.

34. The oral drug dosage form of any one of claims 1-5, further comprising a second swellable material contained, at least in part, in a second swellable material compartment formed by the body, wherein at least one of the moveable arms is configured such that at least a portion of the moveable arm is extendible beyond, or further away from, or rotate around the body, the body of the oral drug dosage form via a force that is provided by the second swellable material.

35. The oral drug dosage form of any one of claims 1-5, wherein the arm has a thickness of at least about 1 mm.

36. The oral drug dosage form of any one of claims 1-5, further comprising an erodible restrainer configured to inhibit extension of the moveable arm.

37. The oral drug dosage form of claim 36, wherein the erodible restrainer erodes within about 30 minutes following administration to an individual.

38. The oral drug dosage form of any one of claims 1-5, wherein the drug is located in one or more of: the moveable arm and/or the body.

39. The oral drug dosage form of claim 38, wherein the drug is selected from the group consisting of riociguat, aceclofenac, bicalutamide, carbamazepine, carvedilol, clotrimazole, cinnarizine, danazol, dapsone, estradiol, exetimibe glibenclamide, fenofibrate, griseofulvin, ibuprofen, itraconazole, ketoconazole, mefenamic acid, naproxen, nevirapine, nifedipine, nitrofurantoin, nomegestrol acetate, phenytoin sodium salt, piroxicam, praziquantel, rifampicin, sulfamethoxazole, trimethoprim, and verapramil hydrochloride.

40. An oral drug dosage form configured for a gastric retention, the oral drug dosage form comprising:
a swellable material;
a moveable arm configured such that at least a portion of the moveable arm is extendible beyond, or further away from, a body of the oral drug dosage form via a force that is provided by the swellable material;
the body comprising:
a swellable material compartment configured to contain at least a portion of the swellable material;
a directional channel operably connected to the swellable material compartment,
wherein the moveable arm comprises a contact element configured to interface with the swellable material in proximity to the directional channel,
wherein the swellable material compartment and the contact element of the moveable arm substantially surround the swellable material, and wherein the moveable arm, or a portion thereof, is configured to slide within the directional channel such that the moveable arm extends beyond, or further away from, the body of the oral drug dosage form along an axis based on the directional channel;
one or more fluid inlets operably connected to the swellable material compartment;
a stop configured to engage with the moveable arm at an extended position; and a drug,
wherein the oral drug dosage form is configured to have a pre-administration state having a compact form and a post-administration state having an extended form providing the gastric retention, and wherein the extended form of the post-administration state of the oral drug dosage form is due to, at least in part, expansion of the swellable material in the presence of a gastrointestinal fluid.

41. The oral drug dosage form of claim 40, wherein the stop comprises a locking element configured to maintain the moveable arm at one or more extended positions.

42. The oral drug dosage form of claim 41, wherein the locking element is configured to maintain the moveable arm at a single position.

43. The oral drug dosage form of claim 42, wherein the locking element is configured to maintain the moveable arm at a plurality of progressing positions.

44. An oral drug dosage form configured for a gastric retention, the oral drug dosage form comprising:
a swellable material;
a first moveable arm and a second movable arm,
wherein the first moveable arm and the second moveable arm are configured such that at least a portion of each moveable arm is extendible beyond, or further away from, a body of the oral drug dosage form via a force that is provided by the swellable material, and wherein the first moveable arm and the second moveable arm extend in substantially opposite directions along an axis based on the directional channel;
the body comprising:
a swellable material compartment configured to contain at least a portion of the swellable material;
a first directional channel and a second directional channel operably connected to the swellable material compartment,
wherein the first moveable arm comprises a first contact element configured to interface with the swellable material in proximity to the first directional channel,
wherein the second moveable arm comprises a second contact element configured to interface with the swellable material in proximity to the second directional channel,
wherein the swellable material compartment and the first contact element of the first moveable arm and second contact element of the second moveable arm substantially surround the swellable material, and
wherein the first moveable arm, or a portion thereof, is configured to slide within the first directional channel such that the first moveable arm extends beyond, or further away from, the body of the oral drug dosage form along the axis, and
wherein the second moveable arm, or a portion thereof, is configured to slide within the second directional channel such that the second moveable arm extends beyond, or further away from, the body of the oral drug dosage form along the axis;
one or more fluid inlets operably connected to the swellable material compartment;
a first stop configured to engage with the first moveable arm at an extended position;
a second stop configured to engage with the second moveable arm at an extended position; and
a drug,
wherein the oral drug dosage form is configured to have a pre-administration state having a compact form and a post-administration state having an extended form providing the gastric retention, and wherein the extended form of the post-administration state of the oral drug dosage form is due to, at least in part, expansion of the swellable material in the presence of a gastrointestinal fluid.

45. An oral drug dosage form configured for a gastric retention, the oral drug dosage form comprising:
a first swellable material;
a first moveable arm,
wherein the first moveable arm is configured such that at least a portion of the moveable arm is extendible beyond, or further away from, a body of the oral drug dosage form via a force that is provided by the first swellable material, and
wherein the first moveable arm is configured to rotate on a first axis;
a second swellable material;
a second moveable arm,
wherein the second moveable arm is configured such that at least a portion of the moveable arm is extendible beyond, or further away from, the body of the oral drug dosage form via a force that is provided by the second swellable material, and
wherein the second moveable arm is configured to rotate on a second axis;
the body comprising:
a first swellable material compartment configured to contain at least a portion of the first swellable material;
a first directional channel operably connected to the first swellable material compartment,
wherein the first direction channel comprises a curved-shaped channel,
wherein the first moveable arm comprises a first contact element configured to interface with the first swellable material in proximity to the first directional channel,
wherein the first swellable material compartment and the first contact element of the first moveable arm substantially surround the first swellable material, and
wherein the first moveable arm, or a portion thereof, is configured to rotate on the first axis by way of the first contact element sliding in the first direction channel such that the first moveable arm extends beyond, or further away from, the body of the oral drug dosage form,
one or more fluid inlets operably connected to the first swellable material compartment;
a second swellable material compartment configured to contain at least a portion of the second swellable material;
a second directional channel operably connected to the second swellable material compartment,
wherein the second direction channel comprises a curved-shaped channel,
wherein the second moveable arm comprises a second contact element configured to interface with the second swellable material in proximity to the second directional channel,
wherein the second swellable material compartment and the second contact element of the second moveable arm substantially surround the second swellable material, and
wherein the second moveable arm, or a portion thereof, is configured to rotate on the second axis by way of the second contact element sliding in the second direction channel such that the second moveable arm extends beyond, or further away from, the body of the oral drug dosage form,
one or more fluid inlets operably connected to the second swellable material compartment;
a drug,
wherein the oral drug dosage form is configured to have a pre-administration state having a compact form and a post-administration state having an extended form providing the gastric retention, and wherein the extended form of the post-administration state of the oral drug dosage form is due to, at least in part, expansion of the swellable material in the presence of a gastrointestinal fluid.

46. An oral drug dosage form configured for a gastric retention, the oral drug dosage form comprising:
a first swellable material;
a first moveable arm and a second movable arm,
wherein the first moveable arm and the second moveable arm are configured such that at least a portion of each moveable arm is extendible beyond, or further away from, a body of the oral drug dosage form via a force that is provided by the first swellable material, and
wherein the first moveable arm and the second moveable arm extend in substantially opposite directions along an axis based on the directional channel;
a second swellable material;
a third moveable arm,
wherein the third moveable arm is configured such that at least a portion of the third moveable arm is extendible beyond, or further away from, the body of the oral drug dosage form via a force that is provided by the second swellable material, and
wherein the third moveable arm is configured to rotate on a first axis;
a third swellable material;
a fourth moveable arm,
wherein the fourth moveable arm is configured such that at least a portion of the fourth moveable arm is extendible beyond, or further away from, the body of the oral drug dosage form via a force that is provided by the third swellable material, and
wherein the fourth moveable arm is configured to rotate on a second axis;
the body comprising:
a first swellable material compartment configured to contain at least a portion of the first swellable material;
a first directional channel and a second directional channel operably connected to the first swellable material compartment,
wherein the first moveable arm comprises a first contact element configured to interface with the first swellable material in proximity to the first directional channel,
wherein the second moveable arm comprises a second contact element configured to interface with the first swellable material in proximity to the second directional channel,
wherein the first swellable material compartment and the first contact element of the first moveable arm and second contact element of the second moveable arm substantially surround the first swellable material, and
wherein the first moveable arm, or a portion thereof, is configured to slide within the first directional channel such that the first moveable arm extends beyond, or further away from, the body of the oral drug dosage form along the axis, and
wherein the second moveable arm, or a portion thereof, is configured to slide within the second directional channel such that the second moveable arm extends beyond, or further away from, the body of the oral drug dosage form along the axis;
one or more fluid inlets operably connected to the first swellable material compartment;
a first stop configured to engage with the first moveable arm at an extended position;
a second stop configured to engage with the second moveable arm at an extended position;
a second swellable material compartment configured to contain at least a portion of the second swellable material;
a third directional channel operably connected to the second swellable material compartment,
wherein the third direction channel comprises a curved-shaped channel, wherein the third moveable arm comprises a contact element configured to interface with the second swellable material in proximity to the third directional channel,
wherein the second swellable material compartment and the contact element of the third moveable arm substantially surround the second swellable material, and
wherein the third moveable arm, or a portion thereof, is configured to rotate on the first axis by way of the contact element sliding in the third direction channel such that the third moveable arm extends beyond, or further away from, the body of the oral drug dosage form,
one or more fluid inlets operably connected to the third swellable material compartment;
a third swellable material compartment configured to contain at least a portion of the third swellable material;
a fourth directional channel operably connected to the third swellable material compartment,
wherein the fourth direction channel comprises a curved-shaped channel,
wherein the fourth moveable arm comprises a contact element configured to interface with the third swellable material in proximity to the fourth directional channel,
wherein the third swellable material compartment and the contact element of the fourth moveable arm substantially surround the third swellable material, and
wherein the fourth moveable arm, or a portion thereof, is configured to rotate on the second axis by way of the contact element sliding in the fourth direction channel such that the fourth moveable arm extends beyond, or further away from, the body of the oral drug dosage form, one or more fluid inlets operably connected to the third swellable material compartment; and
a drug,
wherein the oral drug dosage form is configured to have a pre-administration state having a compact form and a post-administration state having an extended form providing the gastric retention, and wherein the extended form of the post-administration state of the oral drug dosage form is due to, at least in part, expansion of the first swellable material, the second swellable material, and the third swellable material in the presence of a gastrointestinal fluid.

47. An oral drug dosage form configured for a gastric retention, the oral drug dosage form comprising:
a swellable material;
a first moveable arm and a second movable arm,
wherein the first moveable arm and the second moveable arm are configured such that at least a portion of each moveable arm is extendible beyond, or further away from, a body of the oral drug dosage form via a force that is provided by the swellable material, and wherein the first moveable arm and the second moveable arm are configured to rotate on a shared axis in opposite directions;
the body comprising:
a swellable material compartment configured to contain at least a portion of the swellable material;
a first directional channel and a second directional channel operably connected to the swellable material compartment,
wherein the first directional channel and the second directional channel are curved-shaped and configured around the shared axis,
wherein the first moveable arm comprises a first contact element configured to interface with the swellable material in proximity to the first directional channel,
wherein the first contact element is rudder-shaped and configured to travel in the first directional channel such that the first moveable arm rotates beyond, or further away from, the body of the oral drug dosage form relative to the shared axis,
wherein the second moveable arm comprises a second contact element configured to interface with the swellable material in proximity to the second directional channel,
wherein the second contact element is rudder-shaped and configured to travel in the second directional channel such that the second moveable arm rotates beyond, or further away from, the body of the oral drug dosage form relative to the shared axis,
one or more fluid inlets operably connected to the swellable material compartment;
a stop configured to engage with the first moveable arm and the second moveable arm at an extended position;
a drug,
wherein the oral drug dosage form is configured to have a pre-administration state having a compact form and a post-administration state having an extended form providing the gastric retention, and wherein the extended form of the post-administration state of the oral drug dosage form is due to, at least in part, expansion of the swellable material in the presence of a gastrointestinal fluid.

48. An oral drug dosage form configured for a gastric retention, the oral drug dosage form comprising:
a swellable material,
wherein the swellable material is wrapped, at least in part, with a semi-permeable membrane;
a first moveable arm, a second movable arm,
wherein the first moveable arm, the second moveable arm, are configured such that each arm rotates on an independent axis in a direction towards a perpendicularly situated plane via a force that is provided by the swellable material; and
a body comprising:
a first point of connection for the first arm, a second point of connection for the second arm,
wherein each point of connection is configured to provide the independent axis for rotation of the first arm, the second arm wherein each moveable arm comprises a contact element configured to interface with the swellable material or the semi-permeable material, and
a first stop, a second stop configured to engage with the first moveable arm, the second moveable arm, respectively, at an extended position, wherein the oral drug dosage form is configured to have a pre-administration state having a compact form and a post-administration state having an extended form providing the gastric retention, and wherein the extended form of the post-administration state of the oral drug dosage form is due to, at least in part, expansion of the swellable material in the presence of a gastrointestinal fluid.

49. The oral drug dosage form of claim 48, wherein the swellable material is positioned on the body such that expansion of the swellable material actuates the first moveable arm, the second moveable arm to an extended position.
